(19) United States
(12) Patent Application Publication
(10) Pub. No.: US 2024/0401101 A1
(43) Pub. Date: Dec. 5, 2024

(54) A LIVE CELL ASSAY TO DETERMINE PERMEABILITY OF TEST MOLECULES TO AND/OR WITHIN THE PEPTIDOGLYCAN SCAFFOLD OF BACTERIA CELLS

(71) Applicant: University of Virginia Patent Foundation, Charlottesville, VA (US)

(72) Inventor: Marcos M. Pires, Crozet, VA (US)

(21) Appl. No.: 18/698,311

(22) PCT Filed: Oct. 4, 2022
(86) PCT No.: PCT/US2022/077525

§ 371 Date: Apr. 3, 2024

Related U.S. Application Data
(60) Provisional application No. 63/262,065, filed on Oct. 4, 2021.

(51) Int. Cl. C12Q 1/02; G01N 33/58

(52) U.S. Cl. CPC C12Q 1/025; G01N 33/582

(57) ABSTRACT

Provided herein is a fluorescence-based assay that reports on the accessibility of molecules to the surface of bacteria.

### CROSS-REFERENCE TO RELATED APPLICATIONS

This application claims the benefit of U.S. Provisional Appl. Ser. No. 63/262,065, filed Oct. 4, 2021, which is incorporated by reference as if fully set forth herein.

### GOVERNMENT GRANT SUPPORT

This invention was made with government support under GM124893 awarded by the National Institutes of Health. The government has certain rights in the invention.

### BACKGROUND

Proteins from bacterial foes, antimicrobial peptides, and host immune proteins must navigate past a dense layer of bacterial surface biomacromolecules to reach the peptidoglycan (PG) layer of Gram-positive bacteria. A subclass of molecules (e.g., antibiotics with intracellular targets) also must permeate through the PG (in a molecular sieving manner) to reach the cytoplasmic membrane.

### SUMMARY

Despite the biological and therapeutic importance of surface accessibility, systematic analyses in live bacterial cells have been lacking. Provided herein is a live cell fluorescence assay that is robust, shows high level of reproducibility, and reports on the permeability of molecules to and within the PG scaffold. Moreover, the work herein shows that teichoic acids impede the permeability of molecules of a wide range of sizes and chemical composition.

One embodiment provides a live cell assay to determine permeability of test molecules to and/or within the peptidoglycan (PG) scaffold of bacteria cells comprising: a) provide live bacteria cells that comprise PG with a reactive epitope; b) contact said cells of a) with one or more test molecules, wherein the one or more test molecules has a reactive handle that reacts with reactive epitope in the PG, wherein the test molecule has a reporter molecule; and c) measure the amount of reporter molecule, wherein an increase in reporter molecule levels as compared to a control where the cells where not contacted with the test molecule correlates with permeation of said one or more test molecules to and/or within the PG scaffold.

Another embodiment comprises a live cell assay to determine permeability of test molecules to and/or within the peptidoglycan (PG) scaffold of bacteria cells comprising: a) provide live bacteria cells that comprise PG with a reactive handle; b) contact said cells of a) with one or more test molecules, wherein the one or more test molecules has a reactive epitope that binds with reactive handle; c) contact the cells of b) with a reporter molecule that is conjugated to a reactive epitope; and d) measure the amount of reporter molecule, wherein a decrease in reporter molecule levels as compared to a control where the cells where not contacted with the test molecule correlates with permeation of said one or more test molecules to and/or within the PG scaffold.

In one embodiment, prior to b) the cells are cultured with an inhibitor of wall teichoic acid (WTA) biosynthesis. In one embodiment, the inhibitor is tunicamycin. In one embodiment, prior to b) the cells are cultured with positively charged, branched polyethylenimine (BPEI).

In one embodiment, the PG is covalently linked to the reactive epitope. In one embodiment, the PG is covalently linked to the reactive epitope by culturing said cells with said reactive epitope for a time to allow the cells to incorporate the PG-reactive epitope into the cell's PG scaffold. In one embodiment, the reactive epitope is part of a stem peptide for culturing with said cells. In one embodiment, the stem peptide is 1, 2, 3, 4, 5, 6, 7, 8, 9 or 10 amino acids long and can be used as a PG building block by the cells. In one embodiment, the reactive epitope is on the N-terminus, C-terminus or internal in the stem peptide. In one embodiment, the reactive epitope is D-cysteine or an azide modified D-amino acid, such as D-Lys. In one embodiment the reactive epitope comprises a thiol or azide group.

In one embodiment, reactive handle, test compound or reactive epitope is conjugated to the reporter molecule either directly or by a linker. In one embodiment, the linker is at least one PEG.

In one embodiment, the reporter molecule is a fluorophore. In one embodiment, the fluorophore is fluorescein, AF488, AF647, BODIPY, Cy5, rhodamine 110, TAMRA, Cy5.5, Cy7, Cy7.5 or coumarin.

In one embodiment, wherein the reactive handle is maleimide or DiBenzoCycloOctyne (DBCO). In one the reactive handle is a modified amino acid or stem peptide. In one embodiment, the stem peptide is 1, 2, 3, 4, 5, 6, 7, 8, 9 or 10 amino acids long.

In one embodiment, the stem peptide is 4 amino acids long. In one embodiment, the reactive handle is on the N-terminus, C-terminus or internal in the stem peptide. In one embodiment, one or more of the amino acids is a D-amino acid. In one embodiment, the reactive handle comprises a DBCO.

In one embodiment, the bacteria are gram-positive bacteria, gram-negative bacteria, mycobacteria or a combination thereof. In one embodiment, the gram-positive bacteria are selected from Staphylococcus aureus, Staphylococcus aureus, Streptococcus pneumoniae, Staphylococcus epidermidis, Hay bacillus, Group A streptococcus, Listeria monocytogenes, Enterococcus faecalis, Bacillus cereus, Gardnerella vaginalis, Streptococcus agalactiae, Anthrax bacterium, Micrococcus luteus, Clostridium botulinum, Clostridium tetani, Clostridium perfringens, Enterococcus faecium, Lactococcus lactis, Klebs-Löffler bacillus, Streptococcus mutans, Cutibacterium acnes, Staphylococcus saprophyticus, Lactobacillus acidophilus, Lactiplantibacillus plantarum, Bacillus thuringiensis, Lacticaseibacillus casei, Lacticaseibacillus rhamnosus, Mycoplasma pneumoniae, Staphylococcus haemolyticus, Bacillus megateriu, Ureaplasma urealyticum, Mycoplasma genitalium, Limosilactobacillus reuteri, Alkalihalobacillus clausii, Ureaplasma parvum, Mycoplasma hominis, Lactobacillus gasseri, Bacillus coagulans, Staphylococcus hominis, Staphylococcus lugdunensis, Streptococcus thermophilus, Streptococcus anginosus, Mycobacterium leprae, Streptococcus dysgalactiae, Bifidobacterium longum, Streptococcus bovis, Streptococcus mitis, Aerococcus urinae, Bifidobacterium animalis, Finegoldia magna and/or Staphylococcus capitis. In one embodiment, the gram-negative bacteria are selected from Escherichia coli, Salmonella, Shigella, Enterobacteriaceae, Pseudomonas, Moraxella, Helicobacter, Stenotrophomonas, Bdellovibrio, acetic acid bacteria, Legionella, cyanobacteria, spirochaetes, Neisseria gonorrhoeae, Neisseria meningitidis, Moraxella catarrhalis, Haemophilus influenzae, Klebsiella pneumoniae, Legionella pneumophila, Pseudomonas aeruginosa, Proteus mirabilis, Enterobacter cloacae, Serratia marcescens, Borrelia burgdorferi, Helicobacter pylori, Salmonella enteritidis, Salmonella typhi, and/or Acinetobacter baumannii. In one embodiment, the mycobacteria bacteria are selected from the group consisting of: Mycobacterium tuberculosis (Mtb); Mycobacterium abscessus; Mycobacterium lepar; Mycobacterium marinum; Mycobacterium bovis; Mycobacterium smegmatis; and/or Mycobacterium avium.

One embodiment provides a method to treat a bacterial infection comprising administering one or more test compounds that were determined to permeate to and/or within the PG scaffold to subject in need thereof.

### BRIEF DESCRIPTION OF THE DRAWINGS

FIG. 1. Surface composition can impact accessibility of molecules to the PG scaffold. Schematic representation of the surface composition of S. aureus delineating key biomacromolecules that can potentially impact penetration of molecules.

FIGS. 2A-2C. Bench-marking assays to show accessibility to bacterial PG scaffold. (A) Assay to tag the bacterial PG scaffold with thiol handles followed by a fluorescent probe that contains an orthogonal binding partner for the thiol handle. (B) Chemical structure of PG analogs modified with a cysteine residue and the fluorescent reporter Mal-Fl. (C) Flow cytometry analysis of S. aureus (ATCC 25923) treated overnight with 1 mM of synthetic PG analogs, reduced with DTT (5 mM), and incubated with 25 μM of Mal-Fl. Data are represented as mean+/−SD (n=3). P-values were determined by a two-tailed t-test (* denotes a p-value <0.05, **<0.01, ***<0.001, ns=not significant).

FIGS. 3A-3B. (A) Confocal microscopy of S. aureus or isolated sacculi from S. aureus. S. aureus (ATCC 25923) treated overnight with 1 mM of D-cystine or D-LysFl. Cells treated with D-cystine were reduced with DTT (5 mM) and incubated with 25 μM of Mal-Fl and imaged. Scale bar=5 mm. Cells were either imaged by confocal microscopy or subjected to a secondary step in which the sacculi was isolated and imaged. (B) Flow cytometry analysis of surface labeled S. aureus (ATCC 25923) treated proteinase K (dark bars) or mutanolysin (clear bars). As in (A), surface labeled cells were treated with 1 mM of D-cystine, reduced with DTT (5 mM), and incubated with 25 μM of Mal-Fl. Data are represented as mean+/−SD (n=3). P-values were determined by a two-tailed t-test (* denotes a p-value <0.05, **<0.01, ***<0.001, ns=not significant).

FIGS. 4A-4F. (A) Cartoon representation of the two libraries. (B) Chemical structures of accessibility probes. (C-D) Flow cytometry analysis of WT S. aureus (ATCC 25923) treated overnight with 1 mM of D-cystine, reduced with DTT (5 mM), and incubated with 25 μM of designated accessibility probes. Data are represented as mean+/−SD (n=3). Molecular dynamics simulations of (E) PEG-based and (F) Pro-based accessibility probes in solution. Depicted are overlaid snapshots of every 1-ns during the last 50-ns of the simulation. Yellow represents the maleimide terminus, dark grey represents the spacer regions, and red represents the fluorescein terminus. P-values were determined by a two-tailed t-test (* denotes a p-value <0.05, **<0.01, ***<0.001, ns=not significant).

FIGS. 5A-5B. WT S. aureus (ATCC 25923) cells were incubated with 1 mM of D-cystine (dark bars), co-incubated with tunicamycin (tuni, 0.1 μg/mL) and 1 mM of D-cystine (grey bars), or S. aureus (ΔtarO) were incubated with 1 mM of D-cystine alone (white bars) overnight. Next, cells were reduced with DTT (5 mM), and incubated with 25 μM of designated accessibility probes. Data are represented as mean+/−SD (n=3). P-values were determined by a two-tailed t-test (* denotes a p-value <0.05, **<0.01, ***<0.001, ns=not significant).

FIGS. 6A-6D. (A) Chemical structures of D-LysAz, DBCO-Fl, and DBCO-pegₙ-Fl. (B) Schematic diagram of the PG labeling of live cells. (C) Flow cytometry analysis of WT S. aureus (ATCC 25923) treated overnight with 1 mM of D-cystine or D-LysAz followed by a treatment with either 25 μM of Mal-Fl or DBCO-Fl. (D) WT S. aureus (ATCC 25923) cells were incubated with 1 mM of D-LysAz alone (dark bars), co-incubated with tunicamycin (tuni, 0.1 μg/mL) and 1 mM of D-LysAz (grey bars), or S. aureus (ΔtarO) were incubated with 1 mM of D-LysAz alone (white bars) overnight. Next, cells were treated with 25 μM of designated accessibility probes. Data are represented as mean+/−SD (n=3). P-values were determined by a two-tailed t-test (* denotes a p-value <0.05, **<0.01, ***<0.001, ns=not significant).

FIGS. 7A-7B. (A) Flow cytometry analysis of WT S. aureus (ATCC 25923) treated overnight with 1 mM of D-LysAz, co-incubated with 0, 32, or 64 μg/mL of BPEI at stationary phase, followed by a treatment with 25 μM of designated probes. (B) Flow cytometry analysis of WT S. aureus (SA546), S. aureus (ΔclpX ΔltaS), or S. aureus (ΔclpX) treated overnight with 1 mM of D-LysAz, followed by a treatment with 25 μM of designated probes. Data are represented as mean+/−SD (n=3). P-values were determined by a two-tailed t-test (* denotes a p-value <0.05, **<0.01, ***<0.001, ns=not significant).

FIG. 8. Modes of incorporation of single amino acids by swapping into the 5^{th} position on the peptidoglycan stem peptide of S. aureus cells. D-cystine is incorporated (singly or doubly) within the PG scaffold and is subsequently reduced with DTT to generate D-cysteine.

FIG. 9. Flow cytometry analysis of S. aureus (ATCC 25923) treated overnight with 1 mM of DMSO/L-cystine/D-cystine, reduced with DTT (5 mM), and incubated with 25 μM of Mal-Fl. Data are represented as mean+/−SD (n=3).

FIG. 10. Flow cytometry analysis of S. aureus (ATCC 25923) treated overnight with 1 mM of D-cystine, reduced with DTT (5 mM), and incubated with increasing concentrations Mal-Fl. Data are represented as mean+/−SD (n=3).

FIGS. 11A-11B. Flow cytometry analysis of S. aureus (ATCC 25923) treated with 500 μg/mL proteinase K (A) or 50 μg/mL mutanolysin (B). S. aureus cells were treated with 1 mM of L-azidohomoalanine and incubated with 25 μM of DBCO-Fl before enzyme treatment. Data are represented as mean+/−SD (n=3).

FIG. 12. Flow cytometry analysis of sacculi isolated from S. aureus (ATCC 25923) treated 500 μg/mL proteinase K (dark bars) or 50 μg/mL mutanolysin (clear bars). S. aureus cells were treated with 1 mM of D-cystine, reduced with DTT (5 mM), and incubated with 25 μM of Mal-Fl prior to sacculi isolation procedure. Data are represented as mean+/−SD (n=3).

FIGS. 13A-13B. Flow cytometry analysis of (A) S. aureus (USA300) and (B) S. aureus (SCO1) treated overnight with 1 mM of D-cystine, reduced with DTT (5 mM), and incubated with designated accessibility probes. Data are represented as mean+/−SD (n=3).

FIG. 14. Summary of the root mean square deviation and radius of gyration for all systems (100 ns).

FIG. 15. Flow cytometry analysis of S. aureus (ATCC 25923) treated overnight with 1 mM of D-cystine or DMSO in the presence of given concentrations of tunicamycin. The next day cells were washed with PBS, reduced with DTT (5 mM), and incubated with 25 μM of Mal-Fl. Data are represented as mean+/−SD (n=3).

FIG. 16. Flow cytometry analysis of S. aureus (ATCC 25923) and S. aureus (ΔtarO) treated overnight with 1 mM of D-cystine or DMSO in the presence of absence of 0.1 μg/L of tunicamycin. The next day cells were washed with PBS, reduced with DTT (5 mM), and incubated with 25 μM of Mal-Fl. Data are represented as mean+/−SD (n=3).

FIGS. 17A-17C. Flow cytometry analysis of S. aureus (ATCC 25923) and S. aureus (ΔtarO) treated overnight with (A) 1 mM of D-cystine or (B) DMSO, reduced with DTT (5 mM), and incubated with designated accessibility probes. Data are represented as mean+/−SD (n=3). (C) Chemical structures of the five fluorophores tested.

FIG. 18. Flow cytometry analysis of surface labeled S. aureus (ATCC 25923) treated overnight with 1 mM of D-LysAz or 1 mM of L-LysAz and incubated with 25 μM of DBCO-Fl. Data are represented as mean+/−SD (n=3).

FIG. 19. Flow cytometry analysis of surface labeled S. aureus (ATCC 25923) treated overnight with 1 mM of D-LysAz and incubated with 25 μM of DBCO-Fl. Cells were washed with PBS and incubated with mutanolysin. Periodically, cells were fixed with formaldehyde and analyzed by flow cytometry. Data are represented as mean+/−SD (n=3).

FIG. 20. Confocal microscopy analysis of surface labeled S. aureus (ATCC 25923) treated overnight with 1 mM of D-LysAz and incubated with 25 μM of DBCO-Fl. Cells were washed with PBS, fixed with formaldehyde, and imaged. Data are represented as mean+/−SD (n=3).

FIG. 21. Flow cytometry analysis of WT S. aureus (ATCC 25923) treated overnight with 1 mM of D-LysAz, co-incubated with 0 or 10 μg/mL of amsacrine overnight, followed by a treatment with 25 μM of designated probes. Data are represented as mean+/−SD (n=3).

FIGS. 22A-22B, 23, 24A-24B, 25A-25B, 26A-26B, 27A-27L, 28A-28N and 29A-29B. HLPC analysis of sample purity.

FIGS. 30A-30B. (A) Schematic representation of the cell surface of mycobacteria. The mycomembrane is generally purported to be the major barrier to the permeation of small molecules into mycobacteria. The peptidoglycan layer is localized past the mycomembrane, thus molecules that reach the peptidoglycan must have permeated through or across the mycomembrane layer. (B) Chemical structure of a canonical pentameric muropeptide of mycobacteria.

FIG. 31. Overall workflow of CAPA. Permeation of azide-displaying small molecules will lead to the click reaction with DBCO imbedded within the PG scaffold of metabolically labeled cells. Lack of permeability will result in high levels of unreacted DBCO whereas high levels of permeation will consume DBCO epitopes. Subsequent treated with an azide-modified fluorescein will reflect the level of unconsumed DBCO when analyzed by flow cytometry.

FIGS. 32A-32B. (A) Spontaneous reaction between DBCO-anchored in the PG and azide displayed within the library members yield a covalent bond. (B) PeT cages the fluorescence of fluorescein, which increases over 20-fold upon reaction with DBCO.

FIGS. 33A-33C. (A) Workflow schematic of the PG labeling with a DBCO-displaying agent followed by treatment with an azide-tagged fluorophore. (B) Chemical structures of D-DapD and TetD. (C) Flow cytometry analysis of Msn treated for 2 h with 50 μM of synthetic PG analogs and incubated with 25 μM of Fl-az. Data are represented as mean+/−SD (n=3). P-values were determined by a two-tailed t-test (* denotes a p-value <0.05, **<0.01, ***<0.001, ns=not significant).

FIGS. 34A-34C. (A) Schematic representation of PAC-MAN. First, mycobacterial cells are metabolically tagged with TetD, followed by an incubation period with the test molecule modified an azide, cells are then incubated with Fl-Az, and finally flow cytometry analysis is performed. (B) Chemical structures of the initial panel of azide-modified molecules that were tested. (C) Flow cytometry analysis of Msn treated for 2 h with 50 μM of synthetic PG analogs, incubated for 2 h with the indicated test molecule, and incubated with 25 μM of Fl-az. Data are represented as mean+/−SD (n=3). P-values were determined by a two-tailed t-test (* denotes a p-value <0.05, **<0.01, ***<0.001, ns=not significant).

FIG. 35. Top: Flow cytometry analysis of Msn treated with 25 μM of synthetic PG analogs and incubated with 50 μM of Fl-az. Data are represented as mean+/−SD (n=3). P-values were determined by a two-tailed t-test (* denotes a p-value <0.05, **<0.01, ***<0.001, ns=not significant). Bottom: Chemical structures of TetD, TetFl, Fl-az, and Fl-acid.

FIG. 36. Flow cytometry analysis of concentration scan of TetD in Msn and incubated with 25 μM of Fl-az for 1 h at 37° C. Data are represented as mean+/−SD (n=3). P-values were determined by a two-tailed t-test (* denotes a p-value <0.05, **<0.01, ***<0.001, ns=not significant).

FIG. 37. Flow cytometry analysis of labeling kinetics of different concentration of Fl-az. Msn was incubated with 25 μM TetD then labeled with Fl-az for different time lengths at 37° C. Samples were then analyzed with flowcytometry. Data are represented as mean+/−SD (n=3).

FIGS. 38A-38B. Flow cytometry analysis of Msn labeled with different dyes. RoAz, 6-azido-rhodamine 110. CoAz, 7-Azido-4-hydroxy coumarin. Msn mc2 155 was incubated with 25 μM TetD then labeled with 25 μM RoAz or CoAz for 1 h at 37° C. Data are represented as mean+/−SD (n=3). P-values were determined by a two-tailed t-test (* denotes a p-value <0.05, **<0.01, ***<0.001, ns=not significant).

FIG. 39. Different Msn strains (mc² 155, ATCC 14468, mc2 1255) were incubated with 25 μM TetD then labeled with 25 μM Fl-Az for 1 h at 37° C. Samples were then analyzed with flowcytometry. Data are represented as mean+/−SD (n=3). P-values were determined by a two-tailed t-test (* denotes a p-value <0.05, **<0.01, ***<0.001, ns=not significant).

FIG. 40. Flow cytometry analysis of meropenem inhibition of incorporation of TetD. Msn was incubated with 25 μM TetD and different concentration of meropenem then labeled with 25 μM Fl-Az for 1 h at 37° C. Data are represented as mean+/−SD (n=3). P-values were determined by a two-tailed t-test (* denotes a p-value <0.05, **<0.01, ***<0.001, ns=not significant).

FIG. 41. Msn mc² 155 and the L,D-transpeptidases knockout train, ldtΔ5, were incubated with 25 μM TetD then labeled with 50 μM Fl-Az for 1 h at 37° C. Samples were then analyzed with flowcytometry. Data are represented as mean+/−SD (n=3). P-values were determined by a two-tailed t-test (* denotes a p-value <0.05, **<0.01, ***<0.001, ns=not significant).

FIG. 42. Confocal microscopy analysis of M. smegmatis metabolically labeled with 25 μM TetD then labeled with 50 μM Fl-Az for 1 h at 37° C.

FIG. 43. Ethidium Bromide and Nile red accumulation analysis. Msn mc2 155 cells were incubated with 25 μM or 50 μM TetD before dispensing to a Costar 96-well half area black opaque flat bottom plate with 5 μM ethidium bromide and 10 μM Nile red in triplicates, respectively. The fluorescent intensity was taken by a Synergy H1 microplate reader for 90 min with 3 min intervals. Wavelengths ethidium bromide, excitation 530 nm, emission 590 nm; Nile red, excitation 540 nm, emission 630 nm.

FIG. 44. Flow cytometry analysis of meropenem inhibition of incorporation of TetD. Msn was incubated with 25 μM TetD and different concentration of LysAz then labeled with 25 μM Fl-Az for 1 h at 37° C. Data are represented as mean+/−SD (n=3). P-values were determined by a two-tailed t-test (* denotes a p-value <0.05, **<0.01, ***<0.001, ns=not significant).

FIG. 45. Mimicking high throughput assay on 96-well plates. Msn was incubated with 25 μM TetD were distributed to 2 96-well plates and treated with PBS or compound 10 in the mini library for 2 h at 37° C. The cells were then labeled with 25 μM Fl-Az for 1 h at 37° C. Data are represented as single dots representing 96 wells (n=96). Top, treatment of compound 10, bottom, treatment with PBS.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a” “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The terms “individual,” “subject,” and “patient,” are used interchangeably herein and refer to any subject for whom diagnosis, treatment, or therapy is desired, including a mammal. Mammals include, but are not limited to, humans, farm animals, sport animals and pets. A “subject” is a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird.

The terms “treatment”, “treating” and the like are used herein to generally mean obtaining a desired pharmacologic and/or physiologic effect, such as arresting or inhibiting, or attempting to arrest or inhibit, the development or progression of a disorder and/or causing, or attempting to cause, the reduction, suppression, regression, or remission of a disorder and/or a symptom thereof. The effect may be prophylactic in terms of completely or partially preventing a disease or symptom thereof and/or may be therapeutic in terms of a partial or complete cure for a disease and/or adverse effect attributable to the disease. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms. Additionally, treatment can be applied to a subject or to a cell culture (in vivo or in vitro).

The terms “inhibit”, “inhibiting”, and “inhibition” refer to the slowing, halting, or reversing the growth or progression of a disease, infection, condition, group of cells, protein or its expression. The inhibition can be greater than about 20%, 40%, 60%, 80%, 90%, 95%, or 99%, for example, compared to the growth or progression that occurs in the absence of the treatment or contacting.

An “effective amount” is an amount sufficient to effect beneficial or desired result, such as a preclinical or clinical result. An effective amount can be administered in one or more administrations. The term “effective amount,” as applied to the compound(s), biologics and pharmaceutical compositions described herein, means the quantity necessary to render the desired therapeutic result. For example, an effective amount is a level effective to treat, cure, or alleviate the symptoms of a disorder and/or disease for which the therapeutic compound, biologic or composition is being administered. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, an “instructional material” includes a publication, a recording, a diagram, or any other medium of expression which can be used to communicate the usefulness of the invention in a kit. The instructional material of the kit of the invention may, for example, be affixed to a container or be provided (such as shipped) together with multiple containers to carry out methods described herein. Alternatively, the instructional material may be provided separately.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard” or “control,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### I. Reactive Epitope/Reactive Handle/Reporter Molecule

Reactive epitopes can be a single amino acid or attached to stem peptide. They can include any D-amino acid, any amino acid in the D-configuration, or a dipeptide with D-stereocenters, or synthetic stem peptide analogs that become recognized by the peptidoglycan biosynthetic machinery. The length of sidechain can be varied because the biosynthetic machinery has wide tolerability for sidechains of various lengths. The D-amino acids include, for example, D-cysteine or modified amino acids, such a modified D-Lys (such as an azide modified D-Lys). The reactive epitope comprises a reactive group, including a one or more thiols and/or one or more azide groups.

The reactive handle can be a modified amino acid or attached to a stem peptide, wherein the reactive handle can comprise maleimide or DiBenzoCycloOctyne (DBCO).

Reporter molecules can comprise one or more fluorophores. Fluorophores include fluorescein, AF488, AF647, BODIPY, Cy5, rhodamine 110, TAMRA, Cy5.5, Cy7, Cy7.5 or coumarin.

### II. Bacteria

The bacteria for use in the methods includes gram-positive bacteria, gram-negative bacteria, mycobacteria or a combination thereof.

The gram-positive bacteria are selected from Staphylococcus aureus, Staphylococcus aureus, Streptococcus pneumoniae, Staphylococcus epidermidis, Hay bacillus, Group A streptococcus, Listeria monocytogenes, Enterococcus faecalis, Bacillus cereus, Gardnerella vaginalis, Streptococcus agalactiae, Anthrax bacterium, Micrococcus luteus, Clostridium botulinum, Clostridium tetani, Clostridium perfringens, Enterococcus faecium, Lactococcus lactis, Klebs-Löffler bacillus, Streptococcus mutans, Cutibacterium acnes, Staphylococcus saprophyticus, Lactobacillus acidophilus, Lactiplantibacillus plantarum, Bacillus thuringiensis, Lacticaseibacillus casei, Lacticaseibacillus rhamnosus, Mycoplasma pneumoniae, Staphylococcus haemolyticus, Bacillus megateriu, Ureaplasma urealyticum, Mycoplasma genitalium, Limosilactobacillus reuteri, Alkalihalobacillus clausii, Ureaplasma parvum, Mycoplasma hominis, Lactobacillus gasseri, Bacillus coagulans, Staphylococcus hominis, Staphylococcus lugdunensis, Streptococcus thermophilus, Streptococcus anginosus, Mycobacterium leprae, Streptococcus dysgalactiae, Bifidobacterium longum, Streptococcus bovis, Streptococcus mitis, Aerococcus urinae, Bifidobacterium animalis, Finegoldia magna and/or Staphylococcus capitis.

The gram-negative bacteria are selected from Escherichia coli, Salmonella, Shigella, Enterobacteriaceae, Pseudomonas, Moraxella, Helicobacter, Stenotrophomonas, Bdellovibrio, acetic acid bacteria, Legionella, cyanobacteria, spirochaetes, Neisseria gonorrhoeae, Neisseria meningitidis, Moraxella catarrhalis, Haemophilus influenzae, Klebsiella pneumoniae, Legionella pneumophila, Pseudomonas aeruginosa, Proteus mirabilis, Enterobacter cloacae, Serratia marcescens, Borrelia burgdorferi, Helicobacter pylori, Salmonella enteritidis, Salmonella typhi, and/or Acinetobacter baumannii.

The mycobacteria bacteria are selected from the group consisting of: Mycobacterium tuberculosis (Mtb); Mycobacterium abscessus; Mycobacterium lepar; Mycobacterium marinum; Mycobacterium bovis; Mycobacterium smegmatis; and/or Mycobacterium avium.

### EXAMPLES

The following examples are provided in order to demonstrate and further illustrate certain embodiments and aspects of the present invention and are not to be construed as limiting the scope thereof.

### Example I

### Introduction

Bacterial cell walls are barriers that protect bacteria against an onslaught of potentially lethal external insults. The therapeutic effectiveness of most antibiotics hinges on their ability to permeate through bacterial surface biomacromolecules to ultimately reach their target. At the same time, bacteria cell wall features have evolved in order to reduce the accessibility of antibacterial agents. For example, S. aureus resistance to vancomycin can result from cell wall thickening, which effectively captures vancomycin molecules and prevents their association with their lipid II target.¹ A molecular sieving concept through the dense cell wall has also been evoked to describe trends in antibacterial activities of synthetic mimics of antimicrobial peptides.² Similarly, central components of the human innate and adaptive immune system, such as lysozyme and antibodies, target cell surface components and do not need to cross the membrane bilayer, yet they too can have their activities modulated by cell surface biopolymers.³⁻⁵

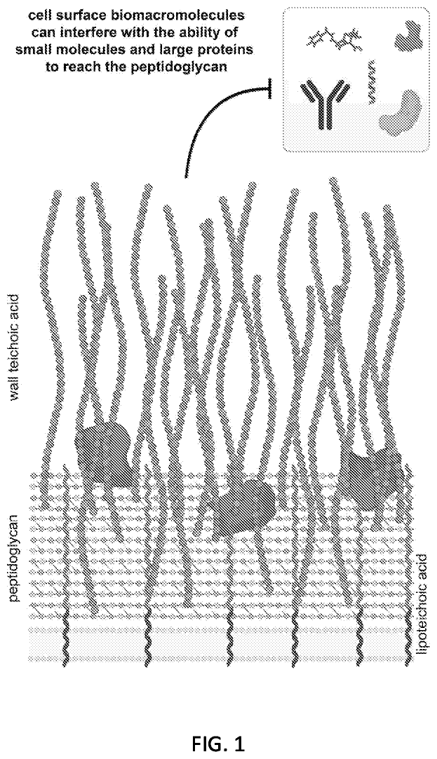

Gram-positive bacteria have a cell wall that includes a thick PG layer on the exterior side of the cytoplasmic membrane (FIG. 1). PG is a mesh-like polymer made up of repeating disaccharides N-acetylglucosamine (GlcNAc) and N-acetylmuramic acid (MurNAc). Each MurNAc unit is connected to a short and unusual peptide (stem peptide) with the canonical sequence of L-Ala-iso-D-Glu-L-Lys-D-Ala-D-Ala or meso-diaminopimelic acid (m-DAP) in place of L-Lys at the 3^{rd} position.⁶ For all known bacteria, neighboring stem peptides are crosslinked to endow the PG matrix with rigidity and integrity. Cell walls are further decorated with a number of polymers and proteins that play important physiological roles such as regulating cell morphology and growth, serving as virulence factors, and aiding in adherence and colonization.⁷ Bacterial PG is a component of the bacterial cell wall, and its unique structure makes it a prominent target for the innate immune system. A significant number of FDA-approved antibiotics also target PG by inhibiting the biosynthesis of it.^{8,9} Given the role of PG in antibacterial therapy, a better understanding of the accessibility to this polymer is imperative.

### Materials and Methods

Flow cytometry analysis of S. aureus treated with thiol analogue panel. LB media containing 1 mM of each respective thiol analogue or dimethyl sulfoxide (DMSO) were prepared. S. aureus ATCC 25923 cells from an overnight culture were added to the medium (1:100 dilution) and allowed to grow overnight at 37° C. with shaking at 250 rpm. The cells were harvested at 4000 rpm and treated with 5 mM dithiothreitol (DTT) at the original culture volume for 5 minutes, to reverse any thiol oxidation that may have occurred. The cells were harvested at 4000 rpm and washed twice at the original culture volume with 1× phosphate-buffered saline (PBS) to remove residual DTT. The cells were then treated with 25 μM FAM maleimide, 6-isomer (Mal-Fl) for 30 minutes at 37° C. and protected from light. The samples were subsequently harvested at 4000 rpm and washed three times with PBS followed by fixation with 2% formaldehyde in 1×PBS for 30 minutes. The cells were washed once more to remove formaldehyde and then analyzed using the Attune NxT flow cytometer equipped with a 488 nm laser and 525/40 nm bandpass filter. The data were analyzed using the Attune NXT Software.

Flow cytometry analysis of S. aureus treated with cystine enantiomers. LB media containing either 1 mM of D-cystine, L-cystine, or DMSO were prepared. S. aureus ATCC 25923 cells from an overnight culture were added to the medium (1:100 dilution) and allowed to grow overnight at 37° C. with shaking at 250 rpm. The cells were harvested at 4000 rpm and treated with 5 mM DTT at the original culture volume for 5 minutes, to reverse any thiol oxidation that may have occurred. The cells were harvested at 4000 rpm and washed twice at the original culture volume with 1×PBS to remove residual DTT. The cells were then treated with 25 μM Mal-Fl for 30 minutes at 37° C. and protected from light. The samples were subsequently harvested at 4000 rpm and washed three times with PBS followed by fixation with 2% formaldehyde in 1×PBS for 30 minutes. The cells were washed once more to remove formaldehyde and then analyzed using the Attune NxT flow cytometer as described above.

Peptidoglycan isolation and confocal microscopy analysis of S. aureus. LB media (25 mL) containing either 1 mM of D-cystine, N^{ε}-azido-D-lysine hydrochloride (D-LysAz), 100 μM D-LysFI, or DMSO were prepared. S. aureus ATCC 25923 cells were added to the LB medium (1:100) and allowed to grow overnight at 37° C. with shaking at 250 rpm. The D-cystine treated cells were harvested at 4000 rpm and treated with 5 mM DTT, to reverse any thiol oxidation that may have occurred, at the original culture volume for 5 minutes. The cells were harvested at 4000 rpm and washed twice at the original culture volume with 1×PBS to remove residual DTT. The cells were then treated with 25 μM Mal-Fl for 30 minutes at 37° C. and protected from light. The samples were subsequently harvested at 4000 rpm and washed three times with 1×PBS. The resulting pellet was resuspended in 1×PBS. Whole cell samples were taken, subjected to fixation with 2% formaldehyde in 1×PBS, and analyzed via confocal microscopy. The remaining sample underwent the peptidoglycan isolation protocol. The D-LysAz treated cells were harvested at 4000 rpm and washed three times at the original culture volume with 1×PBS. The cells were then treated with 25 μM fluorescein-DBCO (DBCO-FI) for 30 minutes at 37° C. and protected from light. The samples were subsequently harvested at 4000 rpm and washed three times with 1×PBS. The resulting pellet was resuspended in 1×PBS. Whole cell samples were taken, subjected to fixation with 2% formaldehyde in 1×PBS, and analyzed via confocal microscopy. The remaining sample underwent the peptidoglycan isolation protocol. The D-LysFI and DMSO treated cells were harvested at 4000 rpm and washed three times at the original culture volume with 1×PBS. Whole cell samples were taken, subjected to fixation with 2% formaldehyde in 1×PBS, and analyzed via confocal microscopy. The remaining sample underwent the peptidoglycan isolation protocol. In order to isolate the peptidoglycan first all four cell suspensions (D-cystine, D-LysAz, D-Lys-FI, and DMSO treated cells) were boiled for 25 minutes to induce cell death. The cells were subsequently harvested at 14000 g. The samples were then treated with 15 mL of 5% sodium dodecyl sulfate (SDS) in deionized water and boiled for 25 minutes. The samples were then sedimented at 14000 g and subjected to treatment with 15 mL of 4% SDS with boiling for 25 minutes. The samples were washed six times with deionized water to remove residual SDS. The resulting pellets were resuspended in 6 mL of 20 mM TRIS HCl at pH8 and treated with 133 μg/mL of DNase in 20 mM TRIS, pH 8, at 37° C. with shaking at 115 rpm for 24 hours. After 24 hours, 133 μg/mL of trypsin in 20 mM TRIS, pH 8, was added to each sample and that was allowed to incubate for 24 hours at 37° C. with shaking at 115 rpm. The samples were then sedimented at 14000 g after the 24-hour time period. Both the whole cell and isolated peptidoglycan samples were analyzed using the Zeiss 980 Airyscan Imaging System provided by the W. M. Keck Center for Cellular Imaging.

Flow cytometry analysis of S. aureus strains treated with Mal-pegₙ-Fl and Mal-proₙ-Fl libraries. LB media containing 1 mM of D-cystine were prepared. S. aureus ATCC 25923, S. aureus ATCC 25923 supplemented with 0.1 μg/mL tunicamycin, S. aureus ΔtarO supplemented with 150 μg/mL spectinomycin, USA300, or S. aureus SCO1 cells from overnight cultures were added to the medium (1:100 dilution) and allowed to grow overnight at 37° C. with shaking at 250 rpm. The cells were harvested at 4000 rpm and treated with 5 mM DTT at the original culture volume for 5 minutes, to reverse any thiol oxidation that may have occurred. The cells were harvested at 4000 rpm and washed twice at the original culture volume with 1×PBS to remove residual DTT. Each strain was then treated with both libraries, Mal-pegₙ-Fl and Mal-proₙ-Fl, in parallel. All library members were used at a 25 μM concentration for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

Flow cytometry analysis of S. aureus strains lacking wall teichoic acids treated with the DBCO-pegₙ-Fl library. LB media containing 1 mM of D-LysAz were prepared. S. aureus ATCC 25923 cells supplemented with 0.1 μg/mL tunicamycin, S. aureus ΔtarO cells supplemented with 150 μg/mL spectinomycin, or S. aureus ATCC 25923 cells from overnight cultures were added to the medium (1:100 dilution) and allowed to grow overnight at 37° C. with shaking at 250 rpm. The cells were harvested at 4000 rpm and washed three times at the original culture volume with 1×PBS. Each strain was then treated with the DBCO-pegₙ-Fl library. All library members were used at a 25 μM concentration for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

Flow cytometry analysis of S. aureus strains treated with the DBCO-pegₙ-Fl library after surface neutralization. LB media containing 1 mM of D-LysAz were prepared. S. aureus ATCC 25923 cells from an overnight culture were added to the medium (1:100 dilution) and allowed to grow overnight at 37° C. with shaking at 250 rpm. Upon reaching stationary phase the cells were harvested at 4000 rpm and washed three times at the original culture volume with 1×PBS. The cells were then resuspended in 1×PBS that contained either DMSO, 32 μg/mL of branched polyethylenimine (BPEI), or 64 μg/mL BPEI. That was allowed to incubate at 37° C. for 30 minutes with shaking at 250 rpm. Subsequently the cells were harvested at 4000 rpm and washed three times with 1×PBS before treatment with the DBCO-pegₙ-Fl library. All library members were used at a 25 μM concentration for 30 minutes at 37° C. and protected from light. The samples were then harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

Flow cytometry analysis of S. aureus strains treated with the DBCO-pegₙ-Fl library. TSB media containing 1 mM of D-LysAz were prepared. S. aureus SA546, ΔclpX, or ΔclpXΔltaS cells from overnight cultures were added to the medium (1:100 dilution) and allowed to grow overnight at 37° C. with shaking at 250 rpm. The cells were harvested at 4000 rpm and washed three times at the original culture volume with 1×PBS. Each strain was then treated with the DBCO-pegₙ-Fl library. All library members were used at a 25 μM concentration for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

Flow cytometry analysis of S. aureus strains treated with the DBCO-pegₙ-Fl library after inhibition of D-alanylation. LB media containing 1 mM of D-LysAz were prepared. S. aureus ATCC 25923 cells supplemented with 10 μg/mL amsacrine or S. aureus ATCC 25923 cells from overnight cultures were added to the medium (1:100 dilution) and allowed to grow overnight at 37° C. with shaking at 250 rpm. The cells were harvested at 4000 rpm and washed three times at the original culture volume with 1×PBS. Each strain was then treated with the DBCO-pegₙ-Fl library. All library members were used at a 25 μM concentration for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

### Results/Discussion

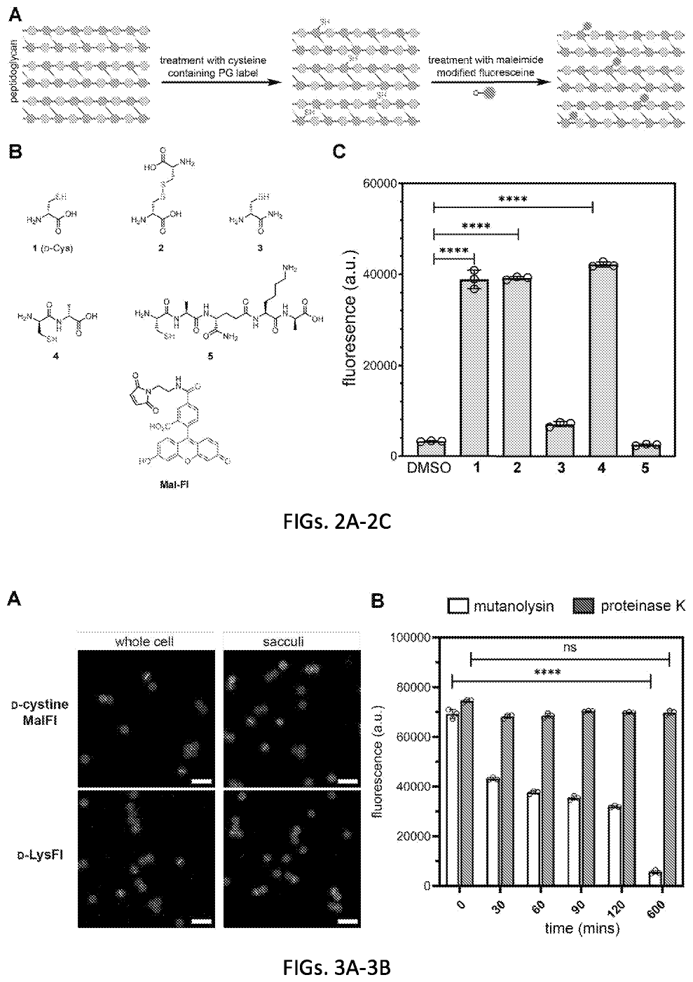

Early works have described a molecular sieving effect of polymers permeating through bacterial PG, which is likely a product of its lattice structure.^{10,11} While illuminating, these experiments were performed in vitro with isolated PG (sacculi). In contrast, herein is provided a method to systematically measure accessibility to the PG scaffold of live bacterial cells. The basis of the assay is a site selective incorporation of a reactive epitope within the PG of live cells followed by treatment with heterobifunctional reporter molecules of varying sizes that attach to the PG scaffold (FIG. 2A). The reporter molecules are linked to fluorophores, and, therefore, cellular fluorescence levels describe the ability of the probes to navigate through surface exposed biomacromolecules. Covalent PG tagging results in reliable measurements that can be readily quantified using standard techniques amendable to high throughput analyses (e.g., flow cytometry). Initially was believed that a thiol functional group could be installed within bacterial PG, which lacks native thiols, by the process of metabolic tagging of the PG with synthetic stem peptide analogs containing D-cysteine.¹²⁻¹⁶ During cell growth and division, synthetic PG analogs enter the biosynthetic pathway in place of endogenous building blocks, and this process provides a robust route to introduce non-native functional groups within bacterial PG.¹⁷

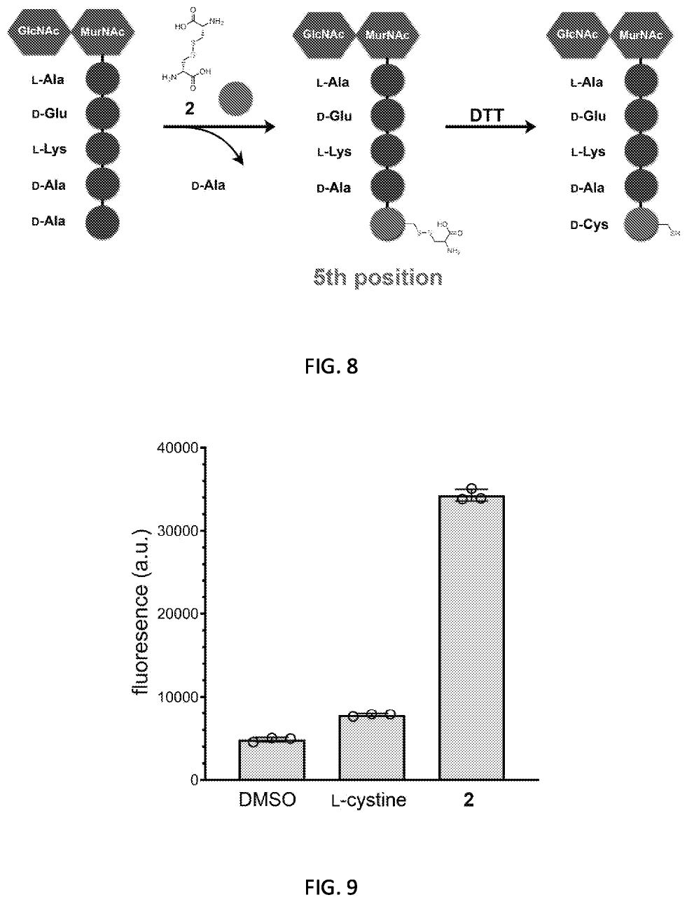

A small panel of synthetic PG analogs was synthesized, each of which contained a cysteine residue (FIG. 2B). The panel contained three derivatives of the single D-amino acid, D-cysteine. During cell growth, single D-amino acids supplemented in the culture medium, such as D-cysteine, are swapped in the place of the D-alanine that occupies the 5^{th} position within the stem peptide of S. aureus (FIG. 8).^{12,18} A wide range of single D-amino acid PG probes have been developed to elucidate fundamental steps in bacterial cell wall biology.¹⁷⁻²⁸ In an attempt to maximize PG tagging, oxidized D-cystine (2) and D-cysteine amidated were also included at the C-terminus (3), which can result in higher levels of unnatural D-amino acid incorporation and/or retention.^{22, 26} Alternatively, cell treatment with the dipeptide D-Cys-D-Ala (4) can result in the incorporation of D-cysteine at the 4^{th} position of the stem peptide.^{12, 29, 30} D-Cys-D-Ala mimics the D-Ala-D-Ala dipeptide PG precursor, therefore it should be processed intracellularly by the MurF enzyme to generate D-cysteine containing PG. Lastly, cysteine was placed at the N-terminus of a tetrapeptide synthetic analog (5) of the PG stem peptide. Here^{28,31}, and others³²⁻³⁵, recently showed that structural analogs of PG stem peptides can be crosslinked into the growing PG scaffold of live cells.

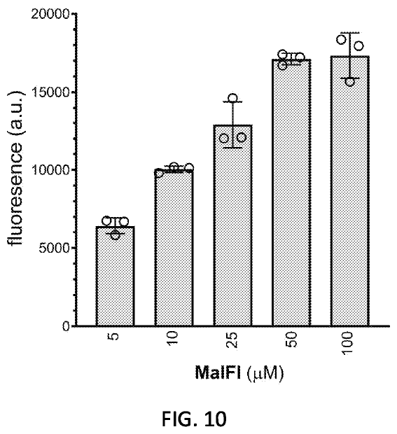

The first goal was to identify which cysteine-based label would result in the highest level of thiol handles on the surface of S. aureus. To accomplish this, S. aureus cells were grown overnight in the presence of each PG analog to promote incorporation throughout the entire PG scaffold. Then, cells were treated with the reducing agent dithiothreitol (DTT) to unmask the thiols on the PG, which were expected to exist primarily as disulfides due to the oxidizing nature of the culture media. Cells were washed with PBS to remove excess reducing agent and incubated with maleimide-modified fluorescein (Mal-Fl). Our results clearly showed that some of the PG metabolic tags resulted in significant increases in fluorescence levels (FIG. 2C). Incubation of cells with D-cystine (2) led to a ˜12-fold increase in cellular fluorescence relative to DMSO treated cells. Interestingly, this large increase suggests that the total amount of cysteines within the modified PG are far greater than the number of cysteines found within endogenous S. aureus cell surface proteins. Likewise, treatment of S. aureus cells with the enantiomeric L-cystine, which is not expected to be processed by PG transpeptidases, led to a minimal increase in cellular fluorescence compared to DMSO treated cells (FIG. 9). Next, a titration experiment was carried out and we found that 25 μM was an optimum concentration of Mal-Fl based on labeling levels (FIG. 10). Although there were similar labeling levels observed with the other PG analogues, we expected that D-cystine, the disulfide form of D-cysteine, would result in more consistent labeling levels compared to the other compounds due to the fact that the others may exist at varying levels of oxidized product during the incubation period in the oxygenated media. It should be noted that larger concentrations of D-amino acids in the culture media (e.g., 100-250 mM D-serine) has resulted in reduced levels of PG crosslinks.^{36, 37} Based on these results, we selected the PG probe D-cystine for all subsequent assays that use the thiol-maleimide pair.

Localization studies were performed next to test whether the Mal-FI is imbedded within the bacterial PG scaffold after reacting with the thiol handle. The laboratory had previously demonstrated that treatment of S. aureus with single D-amino acid probes resulted in the chemical modification of the stem peptide using a range of biochemical techniques.^{12, 18, 25} S. aureus cells were treated with D-cystine followed by Mal-Fl and visualized by confocal microscopy. Our results showed that the reporter probe had similar labeling profile as S. aureus labeled with the single amino acid probe D-LysFl (FIG. 3A). The same cells were subjected to a sacculi isolation procedure and imaged by confocal microscopy as well. Satisfyingly, the pattern of labeling in the isolated PG scaffold mirrored that of the whole cell and are suggestive of the probe being covalently attached to the PG scaffold. A second line of evidence of PG binding by the accessibility probe was provided by treatment of S. aureus with digestion enzymes (FIG. 3B). As before, S. aureus were labeled with D-cystine followed by Mal-Fl, subjected to treatment with either proteinase K or mutanolysin, and cellular fluorescence was measured periodically. Mutanolysin is a muralytic enzyme that cleaves the polysaccharide backbone of PG, thus triggering the release of PG fragments from the cell. Fluorescent probes that are covalently attached to the PG should, likewise, separate from the cells upon treatment with mutanolysin, leading to a reduction in cellular fluorescence levels. As expected, S. aureus cells treated with mutanolysin demonstrated a fluorescence level that was half that of the starting level by 120 minutes. Conversely, fluorescence levels of S. aureus cells treated with proteinase K (a promiscuous protease that typically cleaves the peptide bond adjacent to aliphatic and aromatic amino acids) remained mostly unchanged over the course the entire experiment.

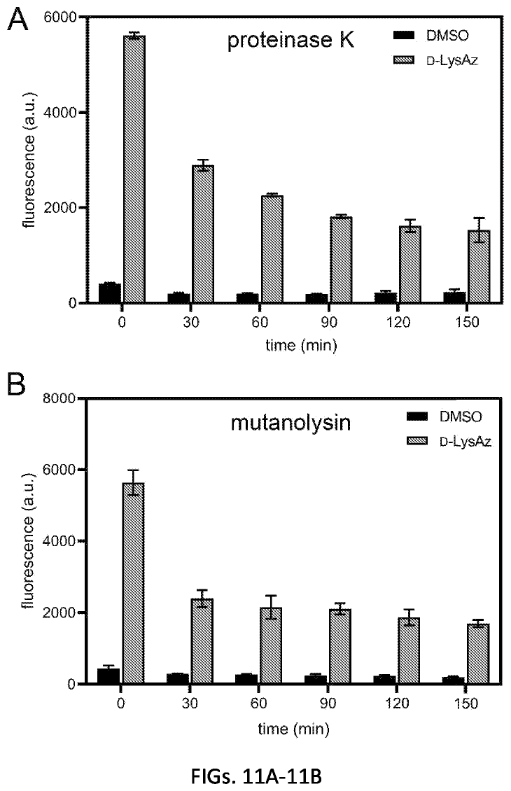

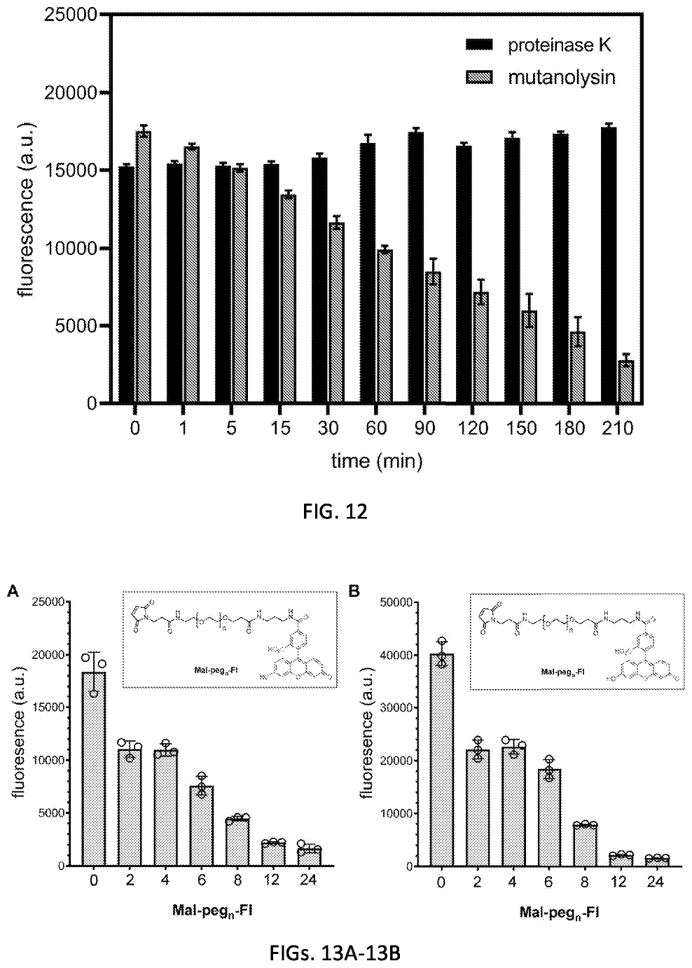

Two additional sets of experiments were performed to confirm the tagging of the PG scaffold by Mal-Fl. First, we performed BioOrthogonal Non-Canonical Amino acid Tagging (BONCAT) of S. aureus (WT) using L-azidohomoalanine (AHA), which is an analog of L-methionine.³⁸⁻⁴⁰ The substrate promiscuity of methionyl-^{t}RNA synthetase allows for the incorporation of AHA into newly synthesized proteins, including those that are surface exposed in S. aureus. We anticipated that, likewise, proteins covalently anchored within the PG by sortase would be readily labeled by AHA. After overnight incubation with AHA, cells were washed and treated with DBCO-Fl. Given the large size of DBCO-Fl, we anticipated that protein tagging would occur primarily (but not exclusively) within surface exposed proteins. Our results showed that treatment with both mutanolysin and proteinase K resulted in a significant decrease in cellular fluorescence (FIG. 11). These results confirm that proteinase K can access, and release proteins tagged with fluorophores. Therefore, the lack of change in fluorescence levels in cells treated with D-cystine followed by Mal-Fl are consistent with PG-specific labeling of D-cystine. Moreover, the isolated sacculus of S. aureus cells, treated with D-cystine followed by Mal-Fl, was analyzed using an assay we recently described (SaccuFlow).⁴¹ SaccuFlow enables the quantification of isolated PG fluorescence. Using this assay, we observed similar sensitivity of PG relative to whole cells upon treatment with mutanolysin and proteinase K (FIG. 12). Together, these results are strongly suggestive of the modification of bacterial PG following the two-step labeling procedure (installation of D-cystine within the PG scaffold followed by covalent attachment of the accessibility probe).

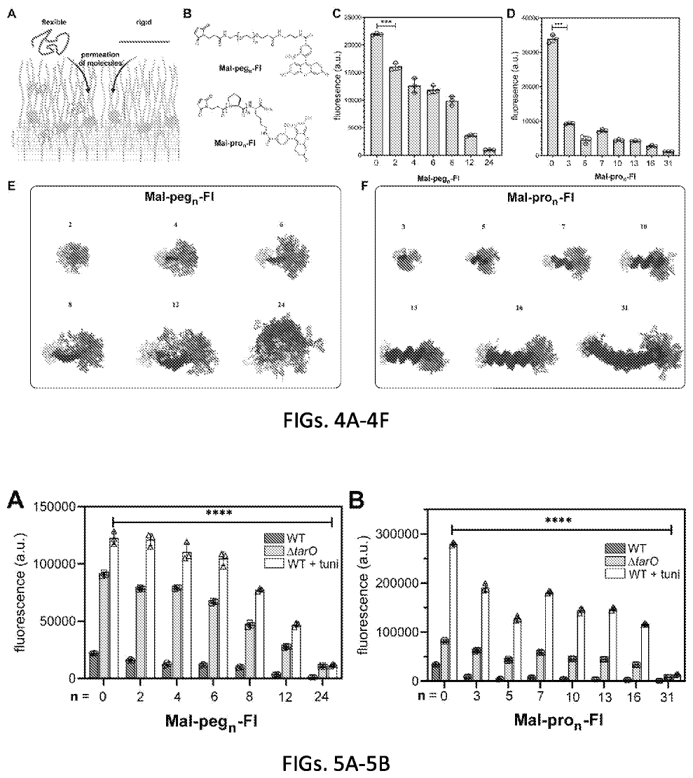

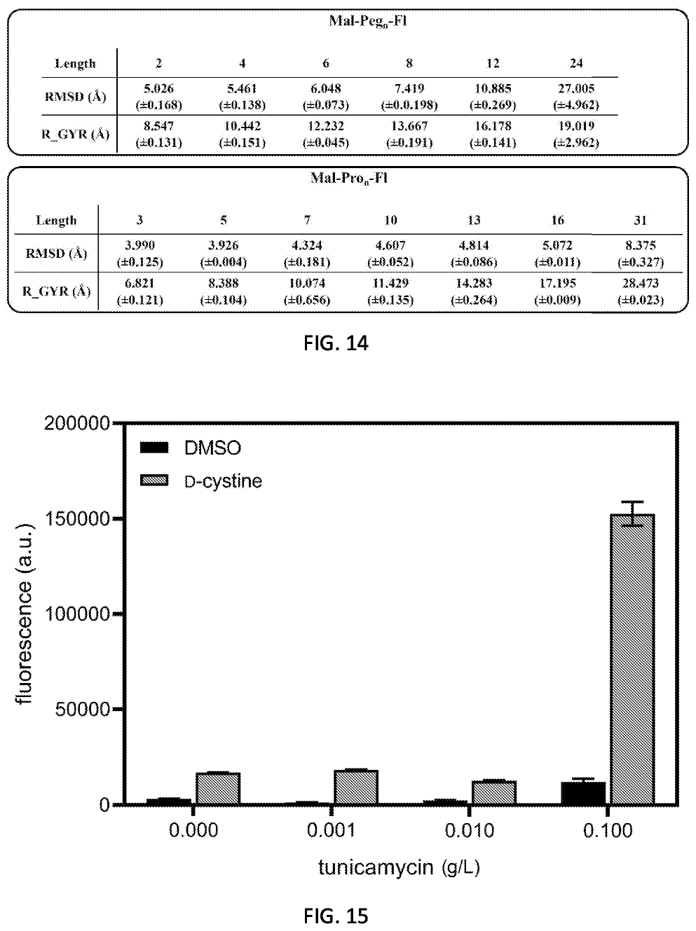

Accessibility to the bacterial PG layer and permeation within the PG scaffold by molecules from the extracellular space should be tied to their physiochemical properties (e.g., charge, size, and flexibility). To test these concepts, we assembled two libraries of accessibility probes that, like Mal-Fl, display maleimide and fluorescein functional groups. One library contained a flexible polar polyethylene glycol (PEG) spacer and while the other was composed of a rigid polyproline spacer, both of varying lengths (FIG. 4 A-B). As before, the PG scaffold of S. aureus cells was tagged with thiol handles by incubating with D-cystine. Accessibility to the PG scaffold was investigated by treating cells with members of both libraries and subsequently analyzing by flow cytometry (FIG. 4 C-D). A stark difference in surface accessibility was noted between the two libraries. Increasing the length of the spacer in Mal-pegₙ-Fl resulted in a gradual and consistent decrease in fluorescence, whereas there was a sharp decrease in cellular fluorescence with the series of Mal-proₙ-Fl. These results indicate that rigidity of a molecule may not be favorable for reaching the PG scaffold of bacteria. Instead, flexibility may promote the maneuvering of molecules across surface biopolymers. A similar labeling profile was observed with two other strains of S. aureus, including the MRSA strain USA300 (FIG. 13). These results likely reflect the conserved nature of D-cystine labeling in these organisms and make this assay applicable to other S. aureus strains. Molecular dynamics (MD) simulations were conducted to determine the conformational variations of the spacer of the Mal-pegₙ-Fl and Mal-proₙ-Fl library members in solution. Snapshots of the last 50-ns in MD simulations of the probes show that Mal-pegₙ-Fl (FIG. 4E) displays considerably more flexibility than Mal-proₙ-Fl (FIG. 4F), which can help explain the results observed in cell surface labeling experiments. More specifically, the root-mean-square deviation (RMSD) values from the equilibrium structure between the shortest spacer and the longest spacer for the Mal-pegₙ-Fl series increases more than 5-fold, whereas for the Mal-proₙ-Fl series there is only a 2-fold increase (FIG. 14; and data not shown).

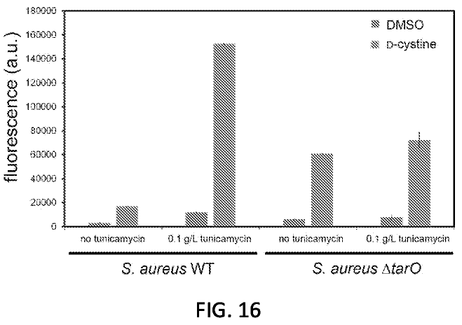

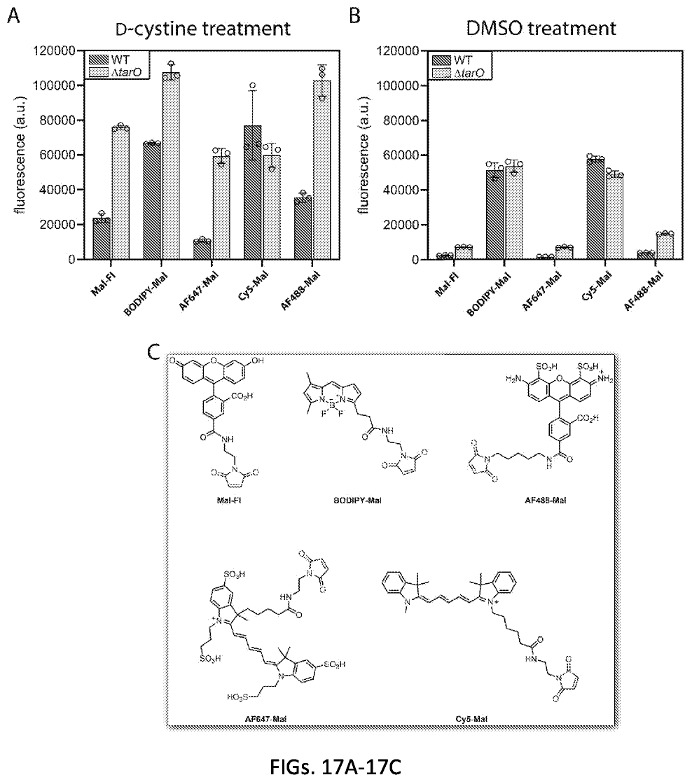

We proceeded to investigate the role of surface biopolymers on PG accessibility. There are two main surface biopolymers on S. aureus cells known as lipoteichoic acids (LTA) and wall teichoic acids (WTA).⁴²⁻⁴⁵ WTA is highly anionic and forms a dense glycan layer that is covalently attached to the stem peptide (FIG. 1). Previous reports have described the influence of WTA on bacteriophage susceptibility,⁴⁶ antibody binding,⁵ antibiotic resistance (e.g., daptomycin),⁴⁷ and recognition by innate immune proteins that bind to PG (e.g., Peptidoglycan Recognition Protein).⁴⁸ Inhibitors of WTA biosynthesis, such as tunicamycin, have been developed as anti-infective agents.⁴⁹⁻⁵² Tunicamycin inhibits TarO, which is responsible for the first step in WTA biosynthesis.⁵³ We sought to gain a more systematic description of the impact of WTA on accessibility to S. aureus PG by using a tarO deletion strain and tunicamycin-based WTA inhibition. Both modes of WTA disruption resulted in large increases in cellular labeling with the accessibility probes (FIG. 5A-B). For example, fluorescence levels in S. aureus (tarO) treated with the 36-atom long spacer, Mal-peg₂-Fl, was higher than S. aureus (WT) treated with Mal-FI. Tunicamycin treatment yielded higher overall levels of cellular fluorescence in a concentration dependent manner (FIG. 15). There was minimal additive effect when ΔtarO cells were treated with tunicamycin (FIG. 16). The boost in cellular fluorescence was more pronounced with rigid spacers as demonstrated by the ratio of cellular fluorescence when treated with Mal-pro₁₃-Fl and Mal-Fl. In the absence of tunicamycin treatment, cellular fluorescence of Mal-pro₁₃-Fl was ˜6.5% relative to Mal-Fl and this ratio jumped to ˜40% upon tunicamycin treatment. Additionally, we also tested a series of maleimide-modified fluorophores in S. aureus (WT) and S. aureus (tarO) to evaluate the role of the fluorophore in the PG labeling (FIG. 17). Our results showed that two other fluorophores (AF488 and AF647) behaved similar to fluorescein in having a low labeling level in the absence of D-amino acid modification and an increase in the absence of WTA in comparison to WT when treated with D-cystine. Two other fluorophores (BODIPY and Cy5) resulted in considerable non-specific labeling.

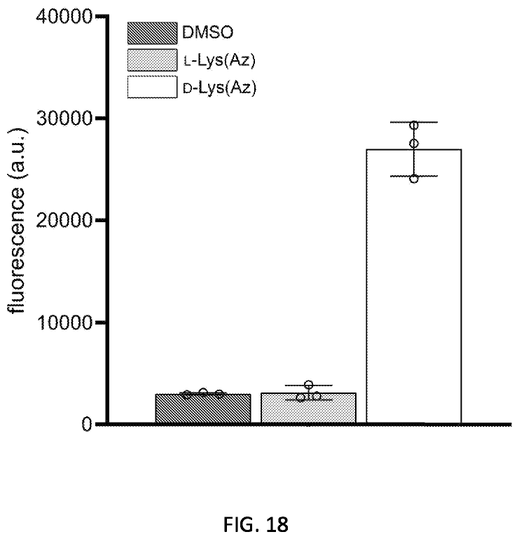

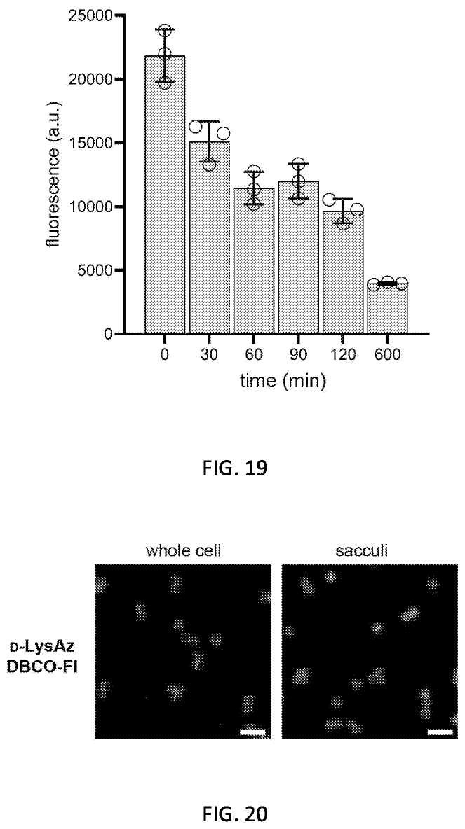

We next set out to test how robust the concept of this assay is by changing the reactive partners. Instead of thiol and maleimide, we assembled a panel of probes centered on an azide-modified D-lysine (D-LysAz) and DiBenzoCycloOctyne (DBCO) conjugated to a fluorescent handle (FIG. 6A).^{54, 55} This pair of reactive functional groups is biorthogonal and readily forms a triazole covalent bond in the absence of metal catalysts. We found that S. aureus cells incubated with D-LysAz followed by treatment with DBCO-Fl resulted in a ˜12-fold increase in cellular fluorescence compared to cells not treated with the unnatural D-amino acid (FIG. 6B). Moreover, the fold increase in cellular fluorescence was very similar to the values observed using the D-cystine and Mal-Fl pair. Cellular treatment with the enantiomer L-LysAz led to background fluorescence levels, another indication that fluorescence signals represent covalent modification of the PG scaffold (FIG. 18). In addition, mutanolysin analysis revealed a similar profile (FIG. 19) and confocal microscopy showed identical localization pattern (FIG. 20) to the thiol-maleimide pair.

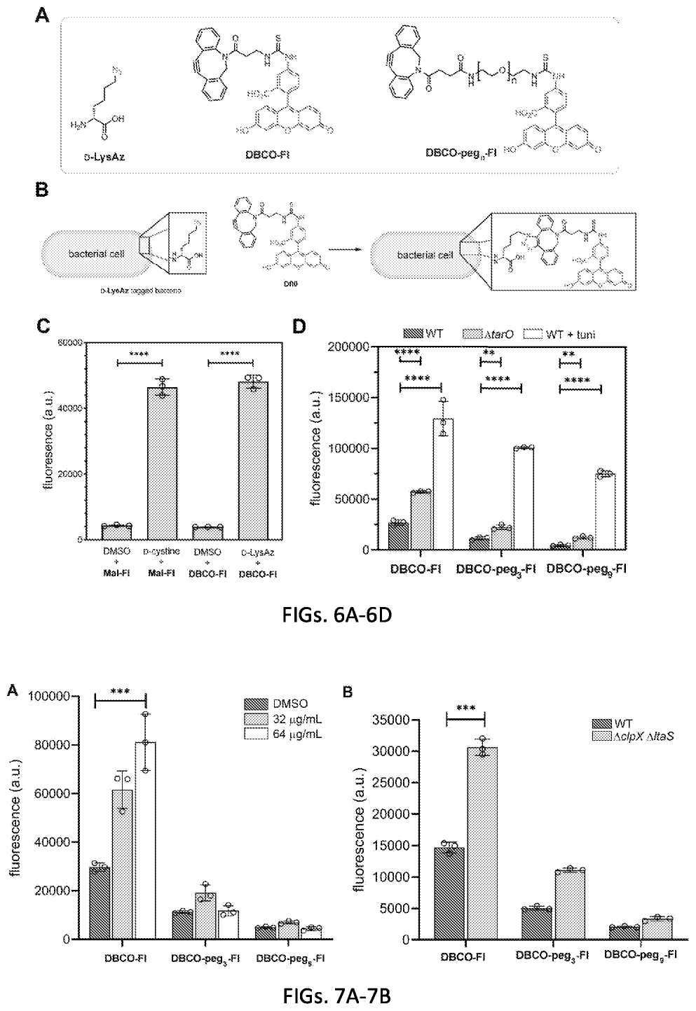

The effect of WTA on PG accessibility was evaluated for the biorthogonal pair (FIG. 6C). Loss of WTA in the deletion strain resulted in a two-fold increase in cellular fluorescence across all spacer lengths. Co-incubation of cells with tunicamycin resulted in ˜9-fold increase in cellular fluorescence with the DBCO-peg₃-Fl probe and a ˜17-fold increase with the longest DBCO-peg₉-Fl probe. These results suggest that the assay platform is robust, and the chemistry is not a factor in measuring access to the PG scaffold. Next, we sought to evaluate the role of positively charged, branched polyethylenimine (BPEI) in potentiating β-lactam antibiotics against MRSA (FIG. 6D).^{56, 57} Whereas tarO-null strain and tunicamycin pre-treatment results in cells lacking WTA, BPEI has been proposed to directly interact with WTA without inhibiting its biosynthesis. Binding of BPEI to WTA was suggested to result in the synergy of β-lactam antibiotics by causing delocalization of penicillin binding proteins. We reasoned that neutralization of WTA by BPEI could also alter accessibility of molecules to the PG scaffold. To test this, S. aureus cells were treated with BPEI and challenged with our probes (FIG. 7A). Our results reveal that BPEI improved the accessibility of smaller molecules to the PG, which likely plays a role in its synergistic activity with small molecule antibiotics. The increased accessibility appeared to be size dependent, as larger molecules did not permeate better when co-incubated with BPEI.

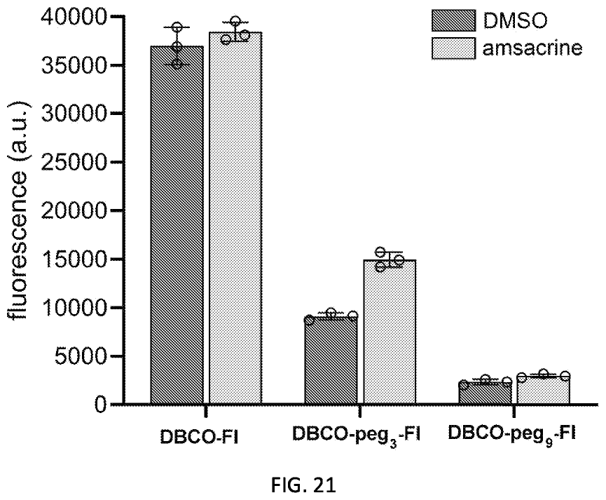

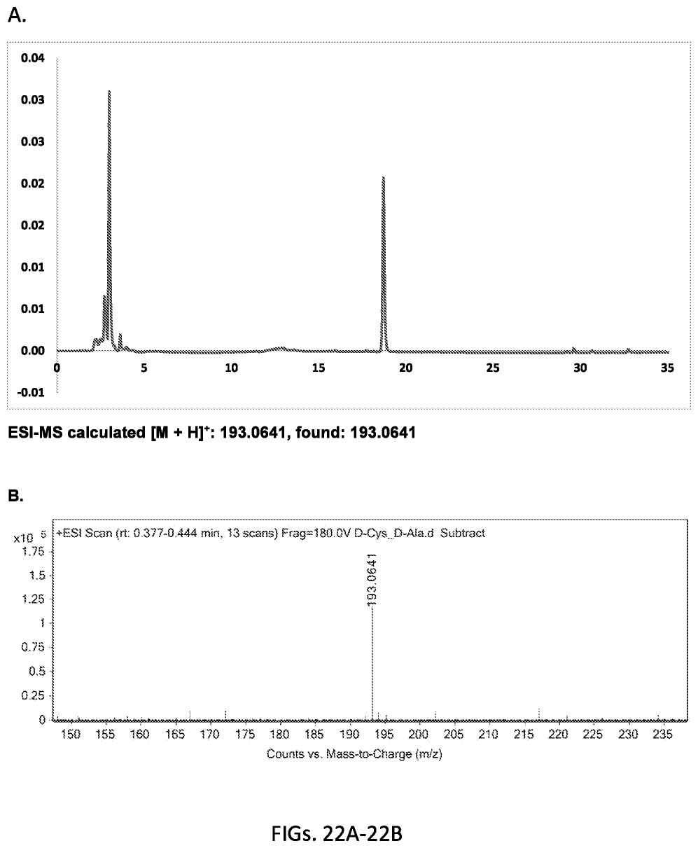

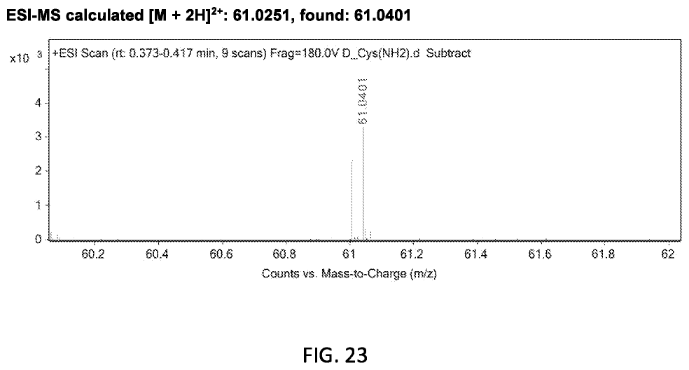

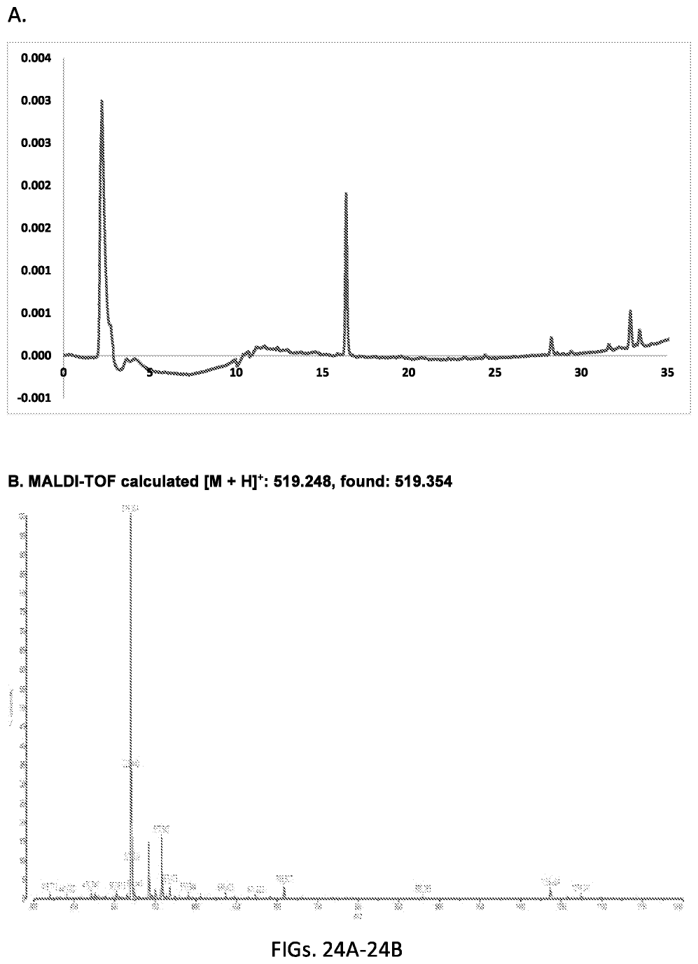

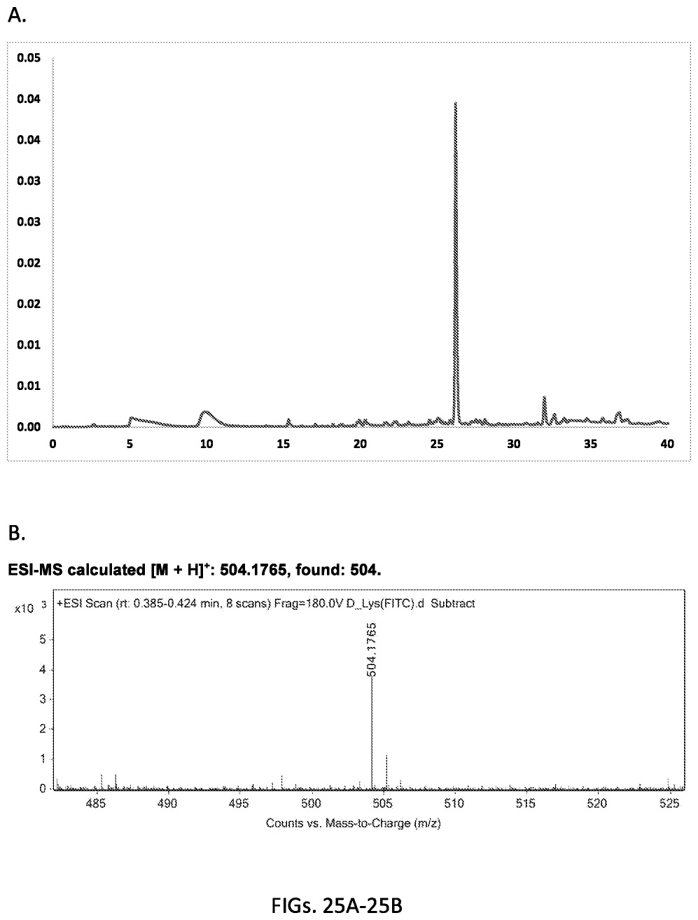

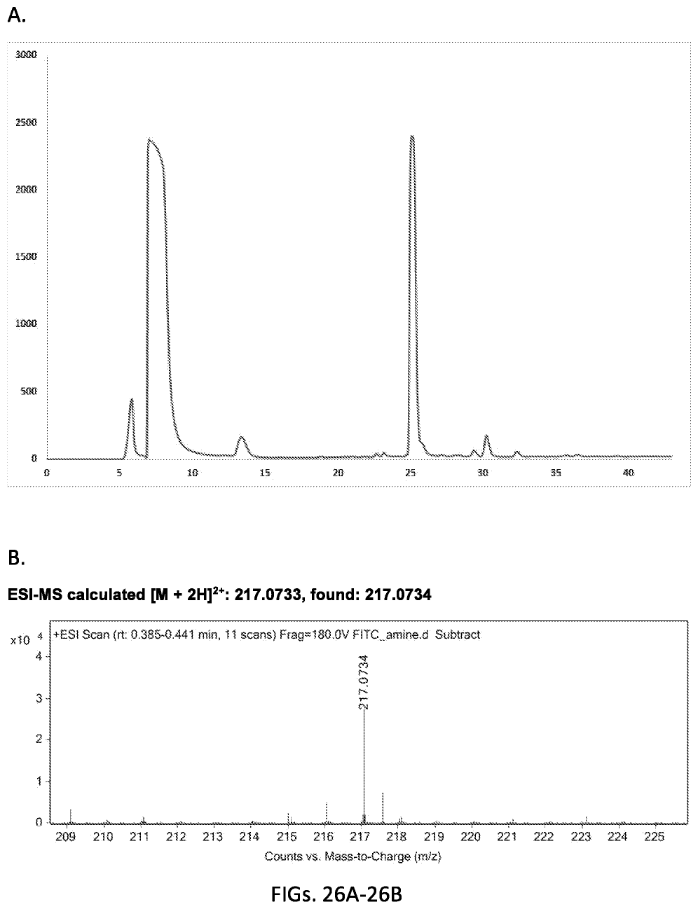

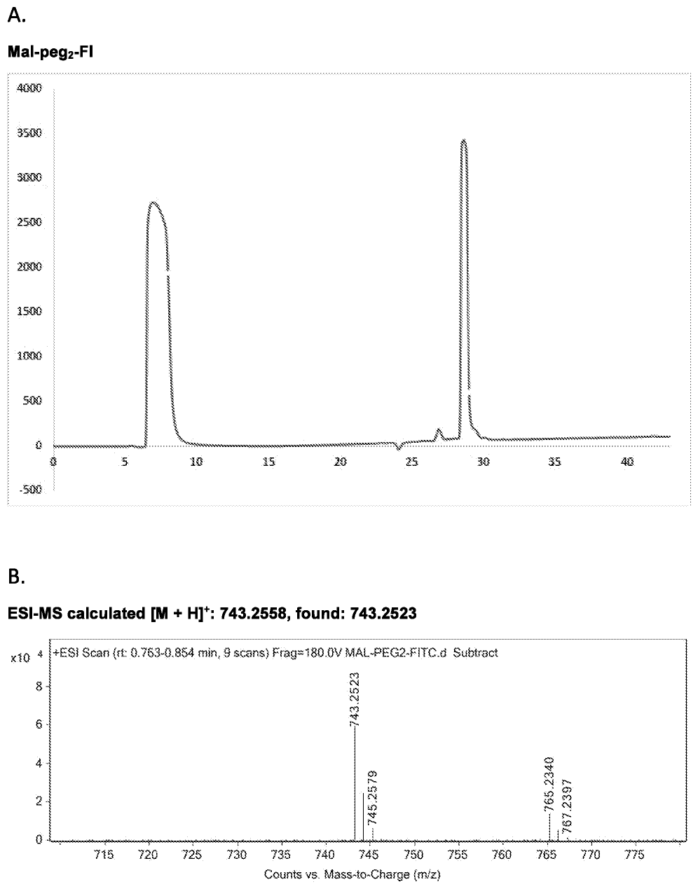

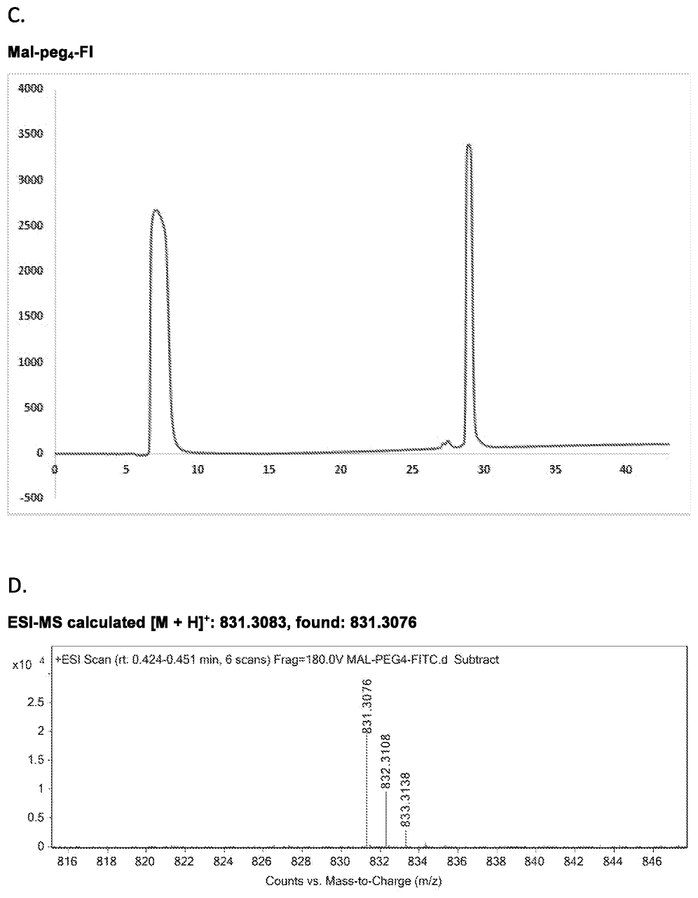

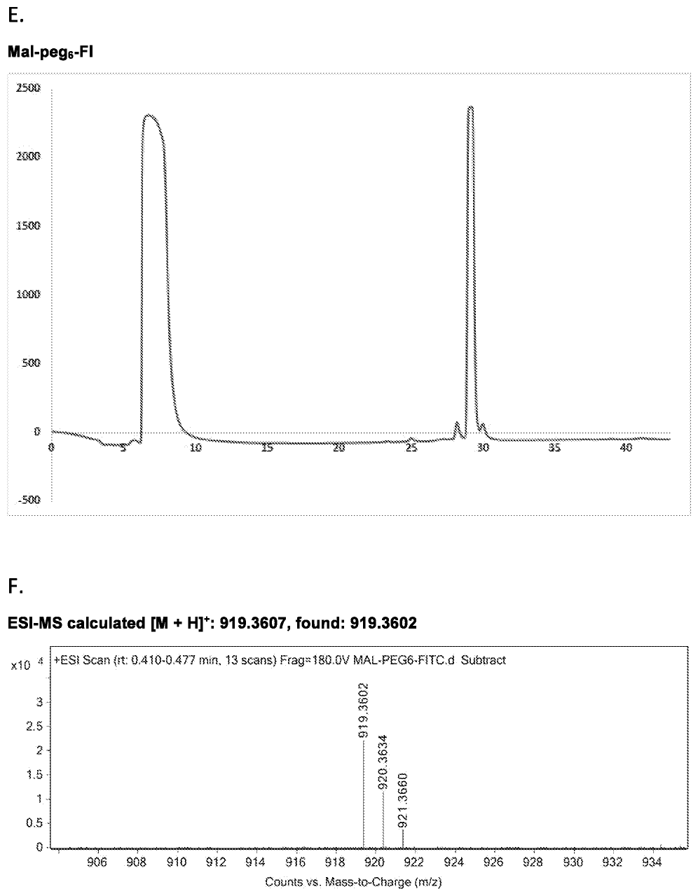

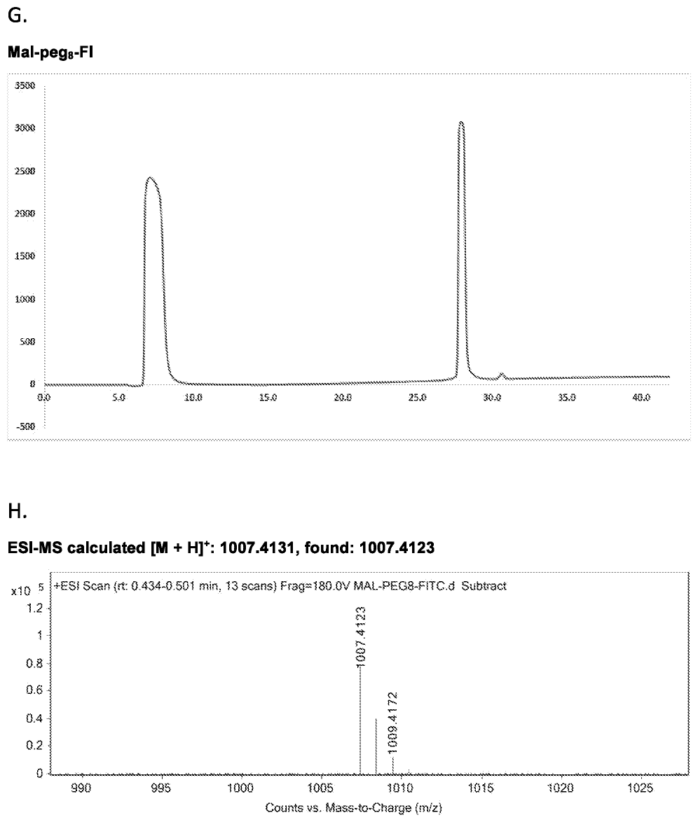

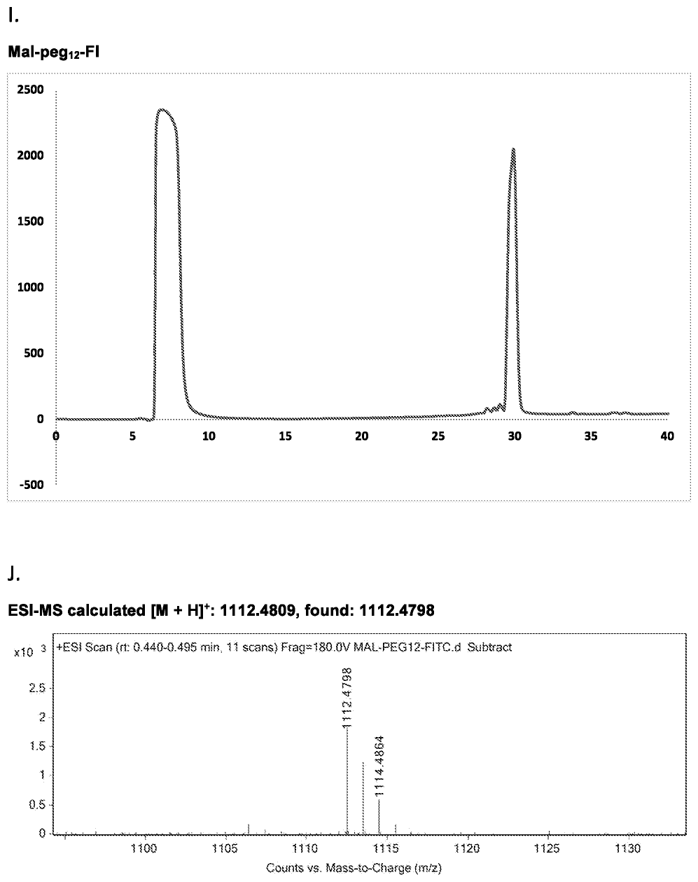

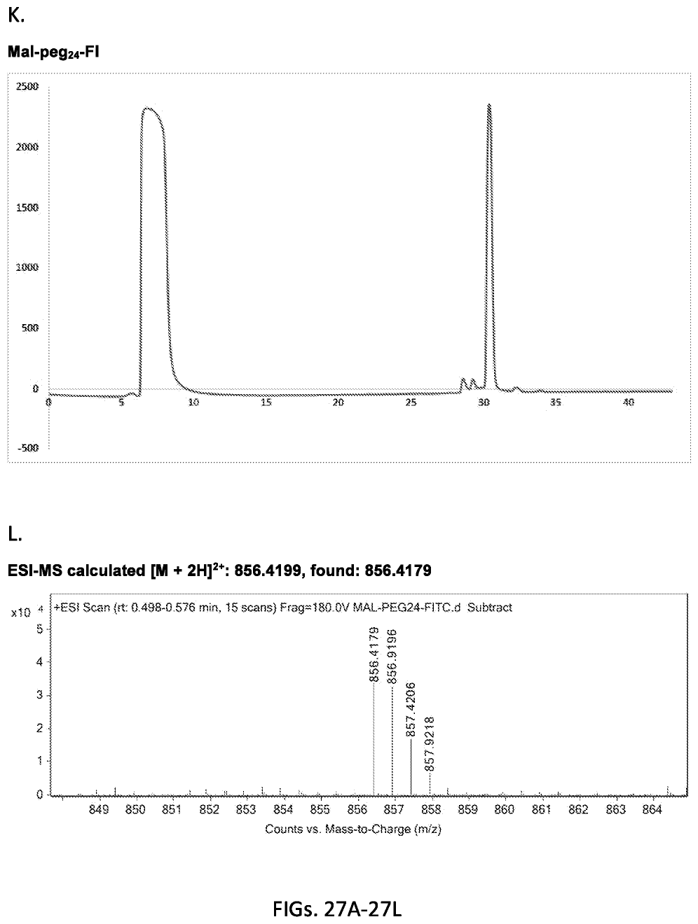

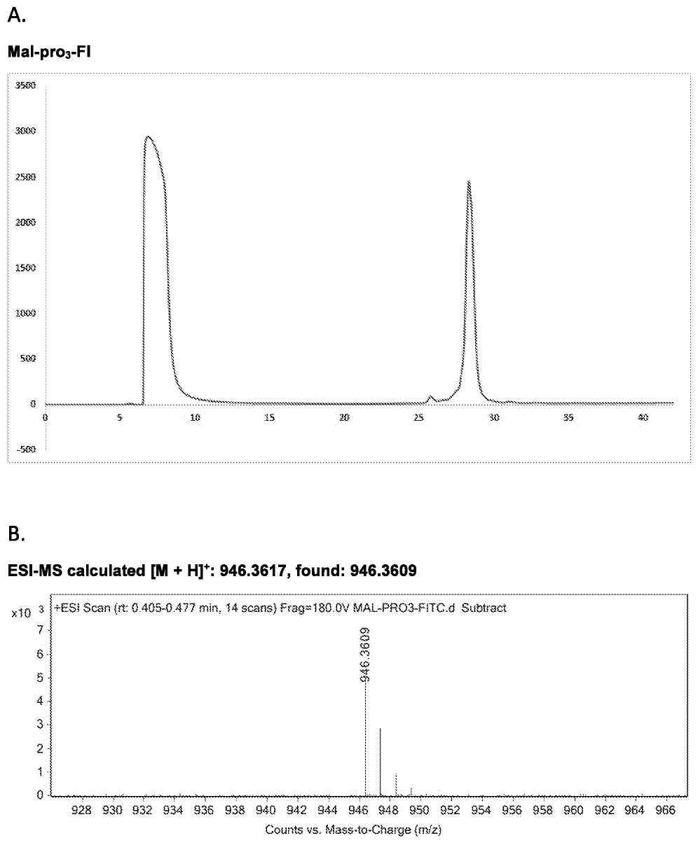

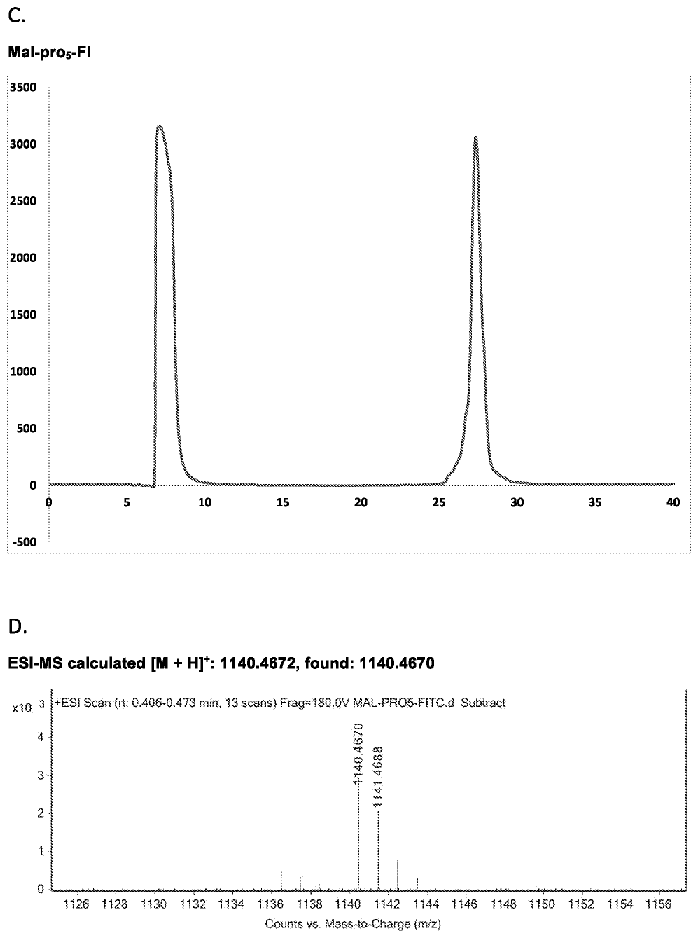

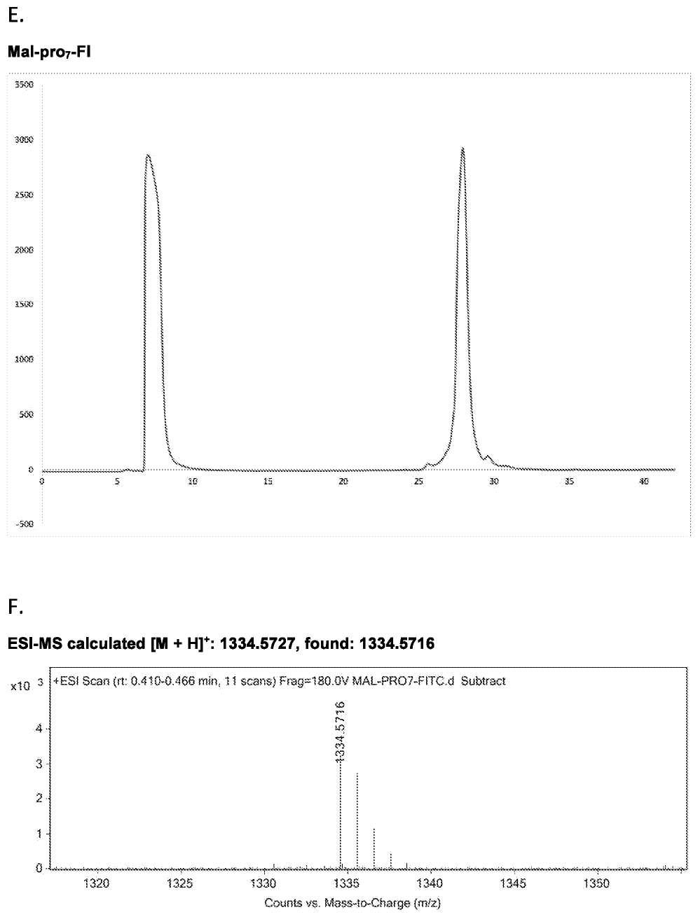

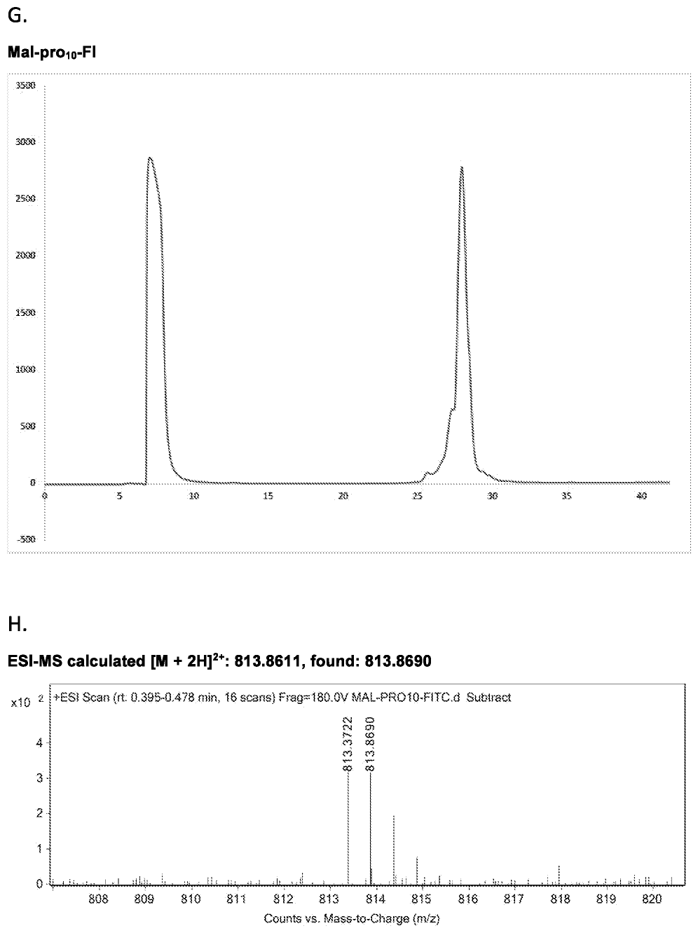

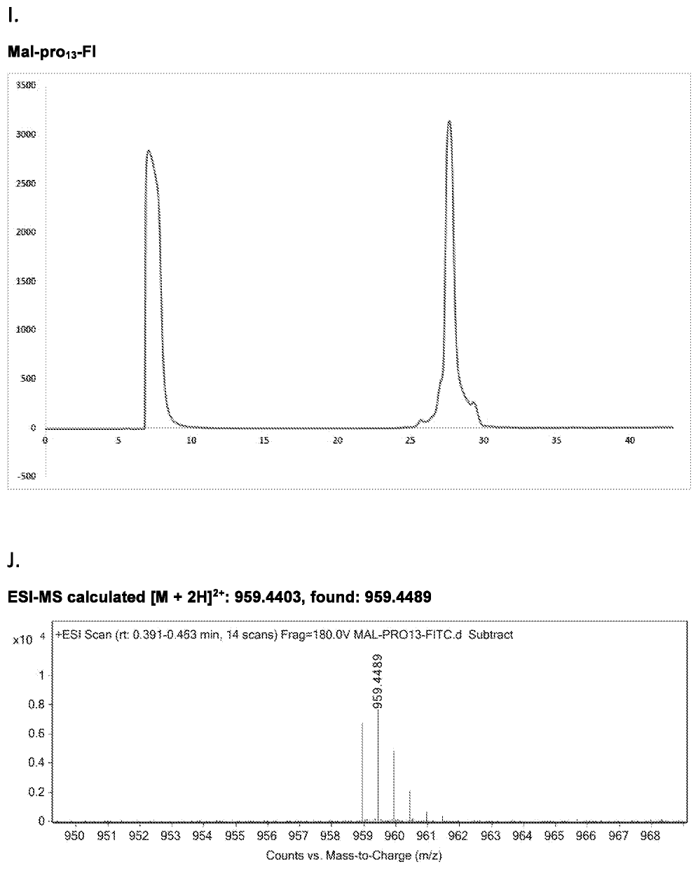

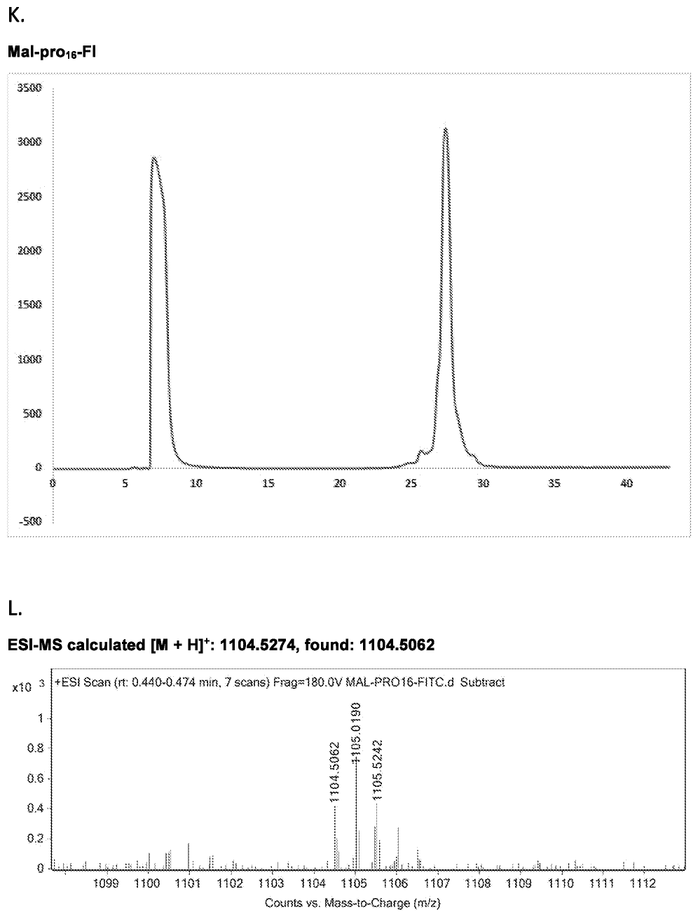

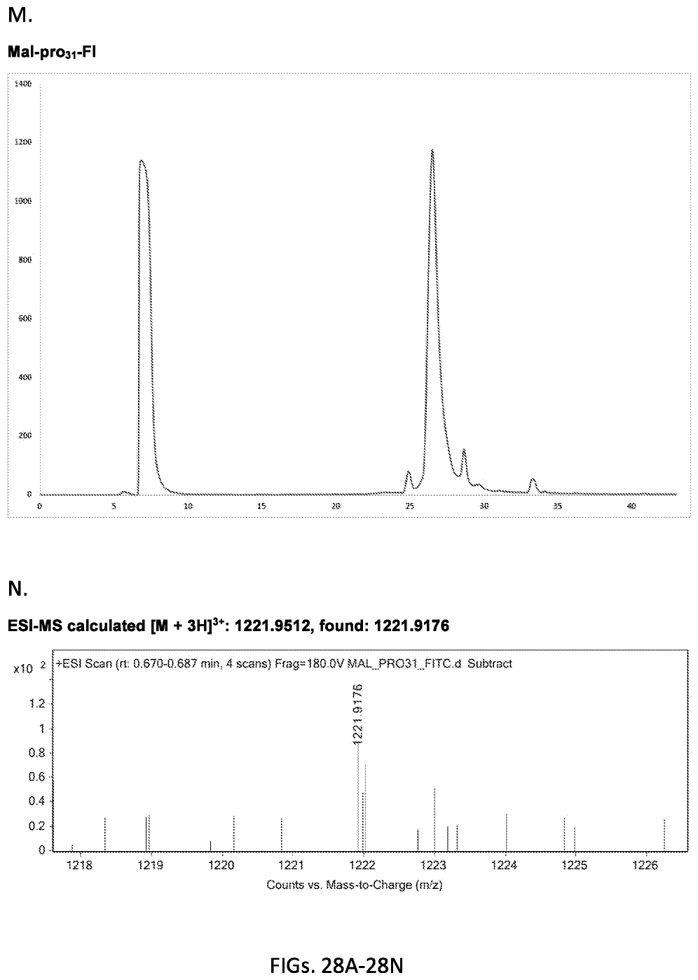

We also set out to assess the effect of LTA on surface accessibility.^{58, 59} Unlike WTA, LTA is anchored into the bacterial membrane via a glycolipid group. Although the roles of LTA have not been fully elucidated, LTA has been implicated in a diverse set of functions including interaction with host toll-like receptors,⁶⁰ organization of cell division machinery,^{61, 62} and regulating biofilm formation.⁶³ The gene responsible for LTA biosynthesis, ltaS, is essential for growth of S. aureus⁶² but becomes conditionally essential when the chaperon ClpX is inactivated.⁶⁴ In our assay, we found that accessibility to the PG of cells lacking LTA was significantly increased (FIG. 7B). Deletion of clpX alone did not lead to an increase in the permeation of DBCO-Fl, indicating that deletion of ClpX alone cannot account for the increased permeability. We then investigated the role of LTA D-alanylation on surface accessibility using a small molecule inhibitor, amsacrine, that was previously described.⁵⁰ Introduction of a positively charged D-alanine within LTA results in the neutralization of the anionic teichoic acids. This modification has been shown to influence a number of biological functions including biofilm formation,^{65, 66} sensitivity to antibiotics (e.g., antimicrobial peptides and daptomycin),^{58, 67-69} and recognition by human innate immune system.⁷⁰ However, its role in surface accessibility has not been evaluated. We found that treatment of S. aureus with amsacrine resulted in a modest increase in cellular fluorescence with DBCO-peg₃-Fl (FIG. 21). Collectively, these results provide direct evidence that chemical or biochemical alterations to teichoic acids on the surface of bacteria can regulate the permeation of molecules. In turn, these results may reveal a new facet to the diverse ways that bacteria modulate access to essential components of the cell wall.

In conclusion, we have developed a novel fluorescence-based assay that reports on the accessibility of molecules to the surface of bacteria. Using S. aureus as a model organism, we showed that two distinct chemical handles (thiol and azide) were installed within the PG scaffold of S. aureus. Using two focused libraries in which each member contained a reactive handle and a fluorophore, we were able to show the effect of molecular size and flexibility on cellular accessibility. Molecules that are rigid, such as polyproline, displayed low access to the bacterial cell surface. Moreover, the presence of WTA (and to a less extent LTA), played a central role in regulating surface accessibility.

Together, these results demonstrate that the assay outlined here is robust, adaptable to different types of Gram-positive bacteria, and will play a significant role in elucidating dynamic features of bacterial cell surfaces.

### BIBLIOGRAPHY

1. Sieradzki, K., Pinho, M. G., Tomasz, A. Inactivated pbp4 in highly glycopeptide-resistant laboratory mutants of Staphylococcus aureus. J. Biol. Chem. 1999, 274, 18942-18946.
2. Lienkamp, K., Kumar, K. N., Som, A., Nusslein, K., Tew, G. N. “Doubly selective” antimicrobial polymers: how do they differentiate between bacteria? Chemistry. 2009, 15, 11710-11714.
3. Mirouze, N., Ferret, C., Cornilleau, C., Carballido-Lopez, R. Antibiotic sensitivity reveals that wall teichoic acids mediate DNA binding during competence in Bacillus subtilis. Nat. Commun. 2018, 9, 5072.
4. Guariglia-Oropeza, V., Helmann, J. D. Bacillus subtilis sigma(V) confers lysozyme resistance by activation of two cell wall modification pathways, peptidoglycan O-acetylation and D-alanylation of teichoic acids. J. Bacteriol. 2011, 193, 6223-6232.
5. Gautam, S., Kim, T., Lester, E., Deep, D., Spiegel, D. A. Wall teichoic acids prevent antibody binding to epitopes within the cell wall of Staphylococcus aureus ACS Chem. Biol. 2016, 11, 25-30.
6. Egan, A. J. F., Errington, J., Vollmer, W. Regulation of peptidoglycan synthesis and remodelling Nat. Rev. Microbiol. 2020, 18, 446-460.
7. Weidenmaier, C., Peschel, A. Teichoic acids and related cell-wall glycopolymers in Gram-positive physiology and host interactions Nat. Rev. Microbiol. 2008, 6, 276-287.
8. Royet, J., Dziarski, R. Peptidoglycan recognition proteins: pleiotropic sensors and effectors of antimicrobial defences Nat. Rev. Microbiol. 2007, 5, 264-277.
9. Wolf, A. J., Underhill, D. M. Peptidoglycan recognition by the innate immune system Nat. Rev. Immunol. 2018, 18, 243-254.
10. Demchick, P., Koch, A. L. The permeability of the wall fabric of Escherichia coli and Bacillus subtilis. J. Bacteriol. 1996, 178, 768-773.
11. Sara, M., Sleytr, U. B. Molecular sieving through S layers of Bacillus stearothermophilus strains. J. Bacteriol. 1987, 169, 4092-4098.
12. Fura, J. M., Pidgeon, S. E., Birabaharan, M., Pires, M. M. Dipeptide-Based Metabolic Labeling of Bacterial Cells for Endogenous Antibody Recruitment. ACS Infect. Dis. 2016, 2, 302-309.
13. Caparros, M., Aran, V., de Pedro, M. A. Incorporation of S-[3H]methyl-D-cysteine into the peptidoglycan of ether-treated cells of Escherichia coli. FEMS Microbiol. Lett. 1992, 72, 139-146.
14. Cava, F., Lam, H., de Pedro, M. A., Waldor, M. K. Emerging knowledge of regulatory roles of D-amino acids in bacteria. Cell Mol. Life Sci. 2011, 68, 817-831.
15. de Pedro, M. A., Quintela, J. C., Holtje, J. V., Schwarz, H. Murein segregation in Escherichia coli. J. Bacteriol. 1997, 179, 2823-2834.
16. Schouten, J. A., Bagga, S., Lloyd, A. J., de Pascale, G., Dowson, C. G., Roper, D. I., Bugg, T. D. Fluorescent reagents for in vitro studies of lipid-linked steps of bacterial peptidoglycan biosynthesis: derivatives of UDPMurNAc-pentapeptide containing d-cysteine at position 4 or 5. Mol. Biosyst. 2006, 2, 484-491.
17. Siegrist, M. S., Swarts, B. M., Fox, D. M., Lim, S. A., Bertozzi, C. R. Illumination of growth, division and secretion by metabolic labeling of the bacterial cell surface. FEMS Microbiol. Rev. 2015, 39, 184-202.
18. Fura, J. M., Kearns, D., Pires, M. M. D-Amino Acid Probes for Penicillin Binding Protein-based Bacterial Surface Labeling. J. Biol. Chem. 2015, 290, 30540-30550.
19. Kuru, E., Hughes, H. V., Brown, P. J., Hall, E., Tekkam, S., Cava, F., de Pedro, M. A., Brun, Y. V., VanNieuwenhze, M. S. In Situ probing of newly synthesized peptidoglycan in live bacteria with fluorescent D-amino acids. Angew. Chem. Int. Ed. Engl. 2012, 51, 12519-12523.
20. Kuru, E., Tekkam, S., Hall, E., Brun, Y. V., Van Nieuwenhze, M. S. Synthesis of fluorescent D-amino acids and their use for probing peptidoglycan synthesis and bacterial growth in situ. Nat. Protoc. 2015, 10, 33-52.
21. Siegrist, M. S., Whiteside, S., Jewett, J. C., Aditham, A., Cava, F., Bertozzi, C. R. (D)-Amino acid chemical reporters reveal peptidoglycan dynamics of an intracellular pathogen. ACS Chem. Biol. 2013, 8, 500-505.
22. Lebar, M. D., May, J. M., Meeske, A. J., Leiman, S. A., Lupoli, T. J., Tsukamoto, H., Losick, R., Rudner, D. Z., Walker, S., Kahne, D. Reconstitution of peptidoglycan cross-linking leads to improved fluorescent probes of cell wall synthesis. J. Am. Chem. Soc. 2014, 136, 10874-10877.
23. Lupoli, T. J., Tsukamoto, H., Doud, E. H., Wang, T. S., Walker, S., Kahne, D. Transpeptidase-mediated incorporation of D-amino acids into bacterial peptidoglycan. J. Am. Chem. Soc. 2011, 133, 10748-10751.
24. Fura, J. M., Sabulski, M. J., Pires, M. M. D-amino acid mediated recruitment of endogenous antibodies to bacterial surfaces. ACS Chem. Biol. 2014, 9, 1480-1489.
25. Pidgeon, S. E., Pires, M. M. Cell Wall Remodeling of Staphylococcus aureus in Live Caenorhabditis elegans. Bioconjug. Chem. 2017, 28, 2310-2315.
26. Pidgeon, S. E., Fura, J. M., Leon, W., Birabaharan, M., Vezenov, D., Pires, M. M. Metabolic Profiling of Bacteria by Unnatural C-terminated D-Amino Acids. Angew. Chem. Int. Ed. Engl. 2015, 54, 6158-6162.
27. Apostolos, A. J., Pidgeon, S. E., Pires, M. M. Remodeling of Cross-bridges Controls Peptidoglycan Cross-linking Levels in Bacterial Cell Walls. ACS Chem. Biol. 2020, 15, 1261-1267.
28. Pidgeon, S. E., Apostolos, A. J., Nelson, J. M., Shaku, M., Rimal, B., Islam, M. N., Crick, D. C., Kim, S. J., Pavelka, M. S., Kana, B. D., Pires, M. M. L,D-Transpeptidase Specific Probe Reveals Spatial Activity of Peptidoglycan Cross-Linking. ACS Chem. Biol. 2019, 14, 2185-2196.
29. Liechti, G. W., Kuru, E., Hall, E., Kalinda, A., Brun, Y. V., VanNieuwenhze, M., Maurelli, A. T. A new metabolic cell-wall labelling method reveals peptidoglycan in Chlamydia trachomatis. Nature. 2014, 506, 507-510.
30. Sarkar, S., Libby, E. A., Pidgeon, S. E., Dworkin, J., Pires, M. M. In Vivo Probe of Lipid II-Interacting Proteins. Angew. Chem. Int. Ed. Engl. 2016, 55, 8401-8404.
31. Apostolos, A. J., Pidgeon, S. E., Pires, M. M. Remodeling of Crossbridges Controls Peptidoglycan Crosslinking Levels in Bacterial Cell Walls. ACS Chem. Biol. 2020.
32. Gautam, S., Kim, T., Shoda, T., Sen, S., Deep, D., Luthra, R., Ferreira, M. T., Pinho, M. G., Spiegel, D. A. An Activity-Based Probe for Studying Crosslinking in Live Bacteria. Angew. Chem. Int. Ed. Engl. 2015, 54, 10492-10496.
33. Gautam, S., Kim, T., Spiegel, D. A. Chemical probes reveal an extraseptal mode of cross-linking in Staphylococcus aureus. J. Am. Chem. Soc. 2015, 137, 7441-7447.
34. Ngadjeua, F., Braud, E., Saidjalolov, S., Iannazzo, L., Schnappinger, D., Ehrt, S., Hugonnet, J. E., Mengin-Lecreulx, D., Patin, D., Etheve-Quelquejeu, M., Fonvielle, M., Arthur, M. Critical Impact of Peptidoglycan Precursor Amidation on the Activity of l,d-Transpeptidases from Enterococcus faecium and Mycobacterium tuberculosis. Chemistry. 2018, 24, 5743-5747.
35. Welsh, M. A., Taguchi, A., Schaefer, K., Van Tyne, D., Lebreton, F., Gilmore, M. S., Kahne, D., Walker, S. Identification of a Functionally Unique Family of Penicillin-Binding Proteins. J. Am. Chem. Soc. 2017, 139, 17727-17730.
36. Pereira, P. M., Filipe, S. R., Tomasz, A., Pinho, M. G. Fluorescence ratio imaging microscopy shows decreased access of vancomycin to cell wall synthetic sites in vancomycin-resistant Staphylococcus aureus. Antimicrob. Agents. Chemother. 2007, 51, 3627-3633.
37. De Jonge, B. L., Gage, D., Xu, N. The carboxyl terminus of peptidoglycan stem peptides is a determinant for methicillin resistance in Staphylococcus aureus. Antimicrob. Agents. Chemother. 2002, 46, 3151-3155.
38. Tom Dieck, S., Muller, A., Nehring, A., Hinz, F. I., Bartnik, I., Schuman, E. M., Dieterich, D. C. Metabolic labeling with noncanonical amino acids and visualization by chemoselective fluorescent tagging. Curr. Protoc. Cell Biol. 2012, Chapter 7, Unit7 11.
39. tom Dieck, S., Kochen, L., Hanus, C., Heumuller, M., Bartnik, I., Nassim-Assir, B., Merk, K., Mosler, T., Garg, S., Bunse, S., Tirrell, D. A., Schuman, E. M. Direct visualization of newly synthesized target proteins in situ. Nat. Methods. 2015, 12, 411-414.
40. Dieterich, D. C., Hodas, J. J., Gouzer, G., Shadrin, I. Y., Ngo, J. T., Triller, A., Tirrell, D. A., Schuman, E. M. In situ visualization and dynamics of newly synthesized proteins in rat hippocampal neurons. Nat. Neurosci. 2010, 13, 897-905.
41. Apostolos, A. J., Ferraro, N. J., Dalesandro, B. E., Pires, M. M. SaccuFlow: A High-Throughput Analysis Platform to Investigate Bacterial Cell Wall Interactions. ACS Infect Dis. 2021, 7, 2483-2491.
42. Atilano, M. L., Pereira, P. M., Yates, J., Reed, P., Veiga, H., Pinho, M. G., Filipe, S. R. Teichoic acids are temporal and spatial regulators of peptidoglycan cross-linking in Staphylococcus aureus. Proc. Natl. Acad. Sci. USA. 2010, 107, 18991-18996.
43. Xia, G., Kohler, T., Peschel, A. The wall teichoic acid and lipoteichoic acid polymers of Staphylococcus aureus. Int. J Med. Microbiol. 2010, 300, 148-154.
44. Swoboda, J. G., Campbell, J., Meredith, T. C., Walker, S. Wall teichoic acid function, biosynthesis, and inhibition. Chembiochem. 2010, 11, 35-45.
45. Santa Maria, J. P., Jr., Sadaka, A., Moussa, S. H., Brown, S., Zhang, Y. J., Rubin, E. J., Gilmore, M. S., Walker, S. Compound-gene interaction mapping reveals distinct roles for Staphylococcus aureus teichoic acids. Proc. Natl. Acad. Sci. USA. 2014, 111, 12510-12515.
46. Eugster, M. R., Loessner, M. J. Wall teichoic acids restrict access of bacteriophage endolysin Ply118, Ply511, and PlyP40 cell wall binding domains to the Listeria monocytogenes peptidoglycan. J. Bacteriol. 2012, 194, 6498-6506.
47. Bertsche, U., Weidenmaier, C., Kuehner, D., Yang, S. J., Baur, S., Wanner, S., Francois, P., Schrenzel, J., Yeaman, M. R., Bayer, A. S. Correlation of daptomycin resistance in a clinical Staphylococcus aureus strain with increased cell wall teichoic acid production and D-alanylation. Antimicrob. Agents Chemother. 2011, 55, 3922-3928.
48. Vaz, F., Kounatidis, I., Covas, G., Parton, R. M., Harkiolaki, M., Davis, I., Filipe, S. R., Ligoxygakis, P. Accessibility to Peptidoglycan Is Important for the Recognition of Gram-Positive Bacteria in Drosophila. Cell Rep. 2019, 27, 2480-2492 e2486.
49. Matano, L. M., Morris, H. G., Hesser, A. R., Martin, S. E. S., Lee, W., Owens, T. W., Laney, E., Nakaminami, H., Hooper, D., Meredith, T. C., Walker, S. Antibiotic That Inhibits the ATPase Activity of an ATP-Binding Cassette Transporter by Binding to a Remote Extracellular Site. J. Am. Chem. Soc. 2017, 139, 10597-10600.
50. Pasquina, L., Santa Maria, J. P., Jr., McKay Wood, B., Moussa, S. H., Matano, L. M., Santiago, M., Martin, S. E., Lee, W., Meredith, T. C., Walker, S. A synthetic lethal approach for compound and target identification in Staphylococcus aureus. Nat. Chem. Biol. 2016, 12, 40-45.
51. Campbell, J., Singh, A. K., Santa Maria, J. P., Jr., Kim, Y., Brown, S., Swoboda, J. G., Mylonakis, E., Wilkinson, B. J., Walker, S. Synthetic lethal compound combinations reveal a fundamental connection between wall teichoic acid and peptidoglycan biosyntheses in Staphylococcus aureus. ACS Chem. Biol. 2011, 6, 106-116.
52. Suzuki, T., Campbell, J., Kim, Y., Swoboda, J. G., Mylonakis, E., Walker, S., Gilmore, M. S. Wall teichoic acid protects Staphylococcus aureus from inhibition by Congo red and other dyes. J. Antimicrob. Chemother. 2012, 67, 2143-2151.
53. Swoboda, J. G., Meredith, T. C., Campbell, J., Brown, S., Suzuki, T., Bollenbach, T., Malhowski, A. J., Kishony, R., Gilmore, M. S., Walker, S. Discovery of a small molecule that blocks wall teichoic acid biosynthesis in Staphylococcus aureus. ACS Chem. Biol. 2009, 4, 875-883.
54. Baskin, J. M., Prescher, J. A., Laughlin, S. T., Agard, N. J., Chang, P. V., Miller, I. A., Lo, A., Codelli, J. A., Bertozzi, C. R. Copper-free click chemistry for dynamic in vivo imaging. Proc. Natl. Acad. Sci. USA. 2007, 104, 16793-16797.
55. Jewett, J. C., Sletten, E. M., Bertozzi, C. R. Rapid Cu-free click chemistry with readily synthesized biarylazacyclooctynones. J. Am. Chem. Soc. 2010, 132, 3688-3690.
56. Hill, M. A., Lam, A. K., Reed, P., Harney, M. C., Wilson, B. A., Moen, E. L., Wright, S. N., Pinho, M. G., Rice, C. V. BPEI-Induced Delocalization of PBP4 Potentiates beta-Lactams against MRSA. Biochemistry. 2019, 58, 3813-3822.
57. Foxley, M. A., Wright, S. N., Lam, A. K., Friedline, A. W., Strange, S. J., Xiao, M. T., Moen, E. L., Rice, C. V. Targeting Wall Teichoic Acid in Situ with Branched Polyethylenimine Potentiates beta-Lactam Efficacy against MRSA. ACS Med. Chem. Lett. 2017, 8, 1083-1088.
58. Brown, S., Xia, G., Luhachack, L. G., Campbell, J., Meredith, T. C., Chen, C., Winstel, V., Gekeler, C., Irazoqui, J. E., Peschel, A., Walker, S. Methicillin resistance in Staphylococcus aureus requires glycosylated wall teichoic acids. Proc. Natl. Acad. Sci. USA. 2012, 109, 18909-18914.
59. Wood, B. M., Santa Maria, J. P., Jr., Matano, L. M., Vickery, C. R., Walker, S. A partial reconstitution implicates DltD in catalyzing lipoteichoic acid d-alanylation. J. Biol. Chem. 2018, 293, 17985-17996.
60. Schwandner, R., Dziarski, R., Wesche, H., Rothe, M., Kirschning, C. J. Peptidoglycan- and lipoteichoic acid-induced cell activation is mediated by toll-like receptor 2. J. Biol. Chem. 1999, 274, 17406-17409.
61. Oku, Y., Kurokawa, K., Matsuo, M., Yamada, S., Lee, B. L., Sekimizu, K. Pleiotropic roles of polyglycerolphosphate synthase of lipoteichoic acid in growth of Staphylococcus aureus cells. J. Bacteriol. 2009, 191, 141-151.
62. Grundling, A., Schneewind, O. Synthesis of glycerol phosphate lipoteichoic acid in Staphylococcus aureus. Proc. Natl. Acad. Sci. USA. 2007, 104, 8478-8483.
63. Fedtke, I., Mader, D., Kohler, T., Moll, H., Nicholson, G., Biswas, R., Henseler, K., Gotz, F., Zahringer, U., Peschel, A. A Staphylococcus aureus ypfP mutant with strongly reduced lipoteichoic acid (LTA) content: LTA governs bacterial surface properties and autolysin activity. Mol. Microbiol. 2007, 65, 1078-1091.
64. Baek, K. T., Bowman, L., Millership, C., Dupont Sogaard, M., Kaever, V., Siljamaki, P., Savijoki, K., Varmanen, P., Nyman, T. A., Grundling, A., Frees, D. The Cell Wall Polymer Lipoteichoic Acid Becomes Nonessential in Staphylococcus aureus Cells Lacking the ClpX Chaperone. mBio. 2016, 7.
65. Fabretti, F., Theilacker, C., Baldassarri, L., Kaczynski, Z., Kropec, A., Holst, O., Huebner, J. Alanine esters of enterococcal lipoteichoic acid play a role in biofilm formation and resistance to antimicrobial peptides. Infect. Immun. 2006, 74, 4164-4171.
66. Wobser, D., Ali, L., Grohmann, E., Huebner, J., Sakinc, T. A novel role for D-alanylation of lipoteichoic acid of Enterococcus faecalis in urinary tract infection. PLoS One. 2014, 9, e107827.
67. Kristian, S. A., Datta, V., Weidenmaier, C., Kansal, R., Fedtke, I., Peschel, A., Gallo, R. L., Nizet, V. D-alanylation of teichoic acids promotes group a streptococcus antimicrobial peptide resistance, neutrophil survival, and epithelial cell invasion. J. Bacteriol. 2005, 187, 6719-6725.
68. Peschel, A., Otto, M., Jack, R. W., Kalbacher, H., Jung, G., Gotz, F. Inactivation of the dlt operon in Staphylococcus aureus confers sensitivity to defensins, protegrins, and other antimicrobial peptides. J. Biol. Chem. 1999, 274, 8405-8410.
69. Bayer, A. S., Schneider, T., Sahl, H. G. Mechanisms of daptomycin resistance in Staphylococcus aureus: role of the cell membrane and cell wall. Ann. N Y Acad. Sci. 2013, 1277, 139-158.
70. Tabuchi, Y., Shiratsuchi, A., Kurokawa, K., Gong, J. H., Sekimizu, K., Lee, B. L., Nakanishi, Y. Inhibitory role for D-alanylation of wall teichoic acid in activation of insect Toll pathway by peptidoglycan of Staphylococcus aureus. J. Immunol. 2010, 185, 2424-2431.

### Example II

### Materials and Methods

Materials. All peptide related reagents (resin, coupling reagent, deprotection reagent, amino acids, and cleavage reagents) were purchased from ChemImpex or Broad Pharm. Bacterial strains S. aureus ATCC 25923, USA300, and S. aureus SCO1 were grown in lysogeny broth (LB). S. aureus ΔtarO was grown in LB supplemented with 150 μg/mL spectinomycin. S. aureus SA546, ΔclpX, and ΔclpXΔltaS were grown in tryptic soy broth (TSB).

Flow cytometry analysis of S. aureus treated with a Mal-FI titration. LB media containing 1 mM of D-cystine was prepared. S. aureus ATCC 25923 cells from an overnight culture were added to the medium (1:100 dilution) and allowed to grow overnight at 37° C. with shaking at 250 rpm. The cells were harvested at 4000 rpm and treated with 5 mM DTT at the original culture volume for 5 minutes, to reverse any thiol oxidation that may have occurred. The cells were harvested at 4000 rpm and washed twice at the original culture volume with 1×PBS to remove residual DTT. The cells were then treated with either 5, 10, 25, 50, or 100 μM Mal-Fl for 30 minutes at 37° C. and protected from light. The samples were subsequently harvested at 4000 rpm and washed three times with PBS followed by fixation with 2% formaldehyde in 1×PBS for 30 minutes. The cells were washed once more to remove formaldehyde and then analyzed using the Attune NxT flow cytometer as described above.

Enzymatic degradation of Whole Cell Samples. LB media containing either 1 mM of D-cystine or D-LysAz were prepared. S. aureus ATCC 25923 cells were added to the LB medium (1:100) and allowed to grow overnight at 37° C. with shaking at 250 rpm. The D-cystine treated cells were harvested at 4000 rpm and treated with 5 mM DTT, to reverse any thiol oxidation that may have occurred, at the original culture volume for 5 minutes. The cells were harvested at 4000 rpm and washed twice at the original culture volume with 1×PBS to remove residual DTT. The cells were then treated with 25 μM Mal-Fl for 30 minutes at 37° C. and protected from light. The samples were subsequently harvested at 4000 rpm and washed three times with 1×PBS. The D-LysAz treated cells were harvested at 4000 rpm and washed three times at the original culture volume with 1×PBS. The cells were then treated with 25 μM DBCO-FI for 30 minutes at 37° C. and protected from light. The samples were subsequently harvested at 4000 rpm and washed three times with 1×PBS. A zero-time point sample was taken from both the Mal-FI and DBCO-FI treated cells before being subjected to treatment with either 50 μg/mL mutanolysin in 1×PBS or 500 μg/mL proteinase K in 50 mM TRIS HCl with 5 mM calcium chloride at pH 8. A portion of the cells were taken at 30, 60, 90, 120, and 600 minutes. At each time point, the collected bacteria resuspended in a final solution of 1×PBS containing 2% formaldehyde to quench the mutanolysin/proteinase K reaction. The cells were analyzed using the Attune NxT flow cytometer as described above.

BONCAT. LB media containing 1 mM of L-azidohomoalanine was prepared. S. aureus ATCC 25923 cells were added to the LB medium (1:100) and allowed to grow overnight at 37° C. with shaking at 250 rpm. The cells were harvested at 4000 rpm and washed three times with 1×PBS. The cells were then treated with 25 μM DBCO-FI for 30 minutes at 37° C. and protected from light. The samples were subsequently harvested at 4000 rpm and washed three times with 1×PBS. A zero time point sample was taken before being proteinase K in 50 mM TRIS HCl with 5 mM calcium chloride at pH 8. A portion of the cells were taken at 30, 60, 90, 120, and 150 minutes. At each time point, the was resuspended in a final solution of 1×PBS containing 2% formaldehyde to quench the mutanolysin/proteinase K reaction. The cells were analyzed using the Attune NxT flow cytometer as described above.

Enzymatic degradation of sacculi samples. LB media containing 1 mM of D-cystine was prepared. S. aureus ATCC 25923 cells were added to the LB medium (1:100) and allowed to grow overnight at 37° C. with shaking at 250 rpm. The D-cystine treated cells were harvested at 4000 rpm and treated with 5 mM DTT, to reverse any thiol oxidation that may have occurred, at the original culture volume for 5 minutes. The cells were harvested at 4000 rpm and washed twice at the original culture volume with 1×PBS to remove residual DTT. The cells were then treated with 25 μM Mal-Fl for 30 minutes at 37° C. and protected from light. The samples were subsequently harvested at 4000 rpm and washed three times with 1×PBS. The cells were then subjected to the peptidoglycan isolation described above. A zero time point sample of the sacculi was taken before being subjecting the sacculi to treatment with either 50 μg/mL mutanolysin in 1×PBS or 500 μg/mL proteinase K in 50 mM TRIS HCl with 5 mM calcium chloride at pH 8. A portion of the cells were taken at 30, 60, 90, 120, and 150 minutes. At each time point, the was resuspended in a final solution of 1×PBS containing 2% formaldehyde to quench the mutanolysin/proteinase K reaction. The cells were analyzed using the Attune NxT flow cytometer as described above.

Tunicamycin scan. LB media containing 1 mM of D-cystine were prepared. S. aureus ATCC 25923, S. aureus ATCC 25923 supplemented with 0.001, 0.01, or 0.1 μg/mL tunicamycin, S. aureus ΔtarO supplemented with 150 μg/mL spectinomycin, or S. aureus ΔtarO supplemented with 150 μg/mL spectinomycin and 0.1 μg/mL tunicamycin from overnight cultures were added to the medium (1:100 dilution) and allowed to grow overnight at 37° C. with shaking at 250 rpm. The cells were harvested at 4000 rpm and treated with 5 mM DTT at the original culture volume for 5 minutes, to reverse any thiol oxidation that may have occurred. The cells were harvested at 4000 rpm and washed twice at the original culture volume with 1×PBS to remove residual DTT. The cells were then treated with 25 μM Mal-Fl for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

Flow cytometry analysis of S. aureus treated with a series of maleimide-modified fluorophores. LB media containing 1 mM of D-cystine were prepared. S. aureus ATCC 25923 or S. aureus ΔtarO supplemented with 150 μg/mL spectinomycin from overnight cultures were added to the medium (1:100 dilution) and allowed to grow overnight at 37° C. with shaking at 250 rpm. The cells were harvested at 4000 rpm and treated with 5 mM DTT at the original culture volume for 5 minutes, to reverse any thiol oxidation that may have occurred. The cells were harvested at 4000 rpm and washed twice at the original culture volume with 1×PBS to remove residual DTT. The cells were then treated with 25 μM of each of the listed maleimide-modified fluorophores for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

Chemistry comparison. LB media containing either 1 mM of D-cystine or D-LysAz were prepared. S. aureus ATCC 25923 cells were added to the LB medium (1:100) and allowed to grow overnight at 37° C. with shaking at 250 rpm. The D-cystine treated cells were harvested at 4000 rpm and treated with 5 mM DTT, to reverse any thiol oxidation that may have occurred, at the original culture volume for 5 minutes. The cells were harvested at 4000 rpm and washed twice at the original culture volume with 1×PBS to remove residual DTT. The cells were then treated with 25 μM Mal-Fl for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above. Concurrently the D-LysAz treated cells were harvested at 4000 rpm and washed three times at the original culture volume with 1×PBS. The cells were then treated with 25 μM DBCO-FI for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

Flow cytometry analysis of S. aureus treated with azide enantiomers. LB media containing either 1 mM of D-LysAz, N^{ε}-azido-L-lysine hydrochloride (L-LysAz), or DMSO were prepared. S. aureus ATCC 25923 cells were added to the LB medium (1:100) and allowed to grow overnight at 37° C. with shaking at 250 rpm. The cells were harvested at 4000 rpm and washed three times at the original culture volume with 1×PBS. The cells were then treated with 25 μM DBCO-Fl for 30 minutes at 37° C. and protected from light. The samples were subsequently harvested at 4000 rpm and washed three times with 1×PBS followed by fixation with 2% formaldehyde in 1×PBS for 30 minutes. The cells were washed once more to remove formaldehyde and then analyzed using the Attune NxT flow cytometer as described above.

### Computational Methods

Different polymerized states of Mal-proₙ-Fl (n: 3, 5, 7, 10, 13, 16, 31) and Mal-pegₙ-Fl (n: 2, 4, 6, 8, 12, 24) were modeled and simulated to check the influences of the length and type of the spacer groups on the conformational variations of the probes. The force field parameters for maleimide (Mal) and fluorescein (Fl) groups and patches among moieties were generated and assembled by analogy from the CHARMM36 force field.¹⁻³ Each probe was solvated by an appropriate size of TIP3P⁴ box with neutralizing ions (Na⁺) following the CHARMM-GUI Solution Builder protocol.⁵ All simulations were performed using OpenMM-7.4.1 simulation package⁶ and the equilibration and production inputs generated by CHARMM-GUI Input Generator.7 For each system, after short minimization and 125-ps NVT (constant particle number, volume, and temperature) equilibration run, a 100-ns NPT (constant particle number, pressure, and temperature) production simulation was performed at 303.15 K and 1 bar. We performed two independent simulations for each system with different initial velocities to improve sampling and check the convergence.

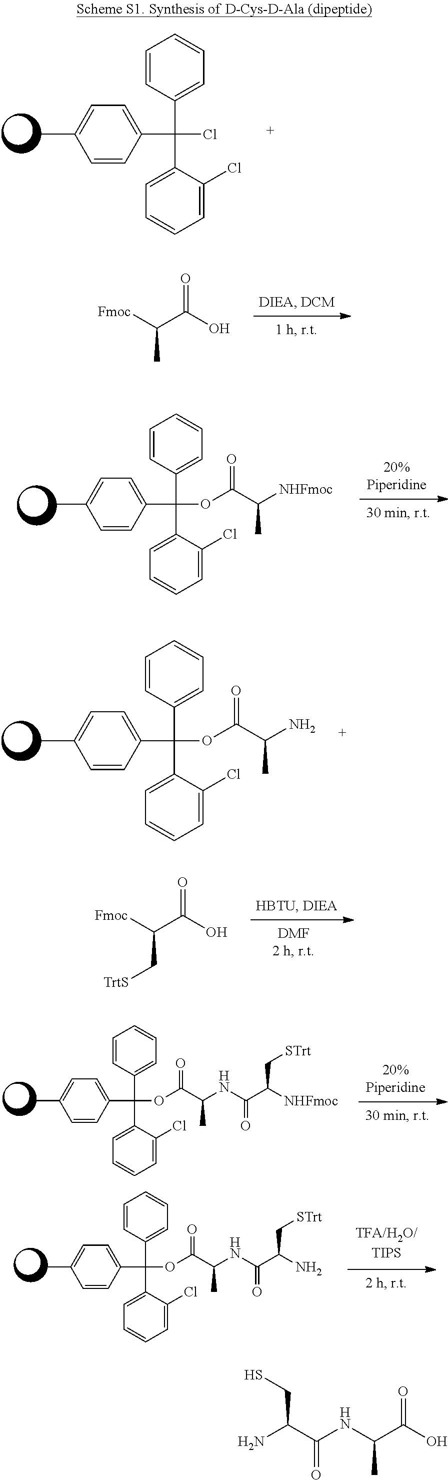

Fmoc-D-Alanine-OH (1.1 eq, 195 mg, 0.62 mmol) was added to a 25 mL peptide synthesis vessel charged with 2-Chlorotrityl chloride resin (500 mg, 0.57 mmol) and DIEA (4.4 eq, 0.436 mL, 2.50 mmol) in dry DCM (5 mL). The resin was agitated for 1 h at ambient temperature and washed with MeOH and DCM (3×15 mL each). The Fmoc protecting group was removed with a 20% piperidine in DMF solution (15 mL) for 30 minutes at ambient temperature, then washed as previously stated. Fmoc-D-Cysteine (Trt)-OH (2 eq, 667 mg, 1.14 mmol), HBTU (1.9 eq, 410 mg, 1.08 mmol), and DIEA (4 eq, 0.397 mL, 2.28 mmol) in DMF (15 mL) were added to the reaction vessel and agitated for 2 h at ambient temperature. After 2 h the resin was washed as previously stated and the Fmoc protecting group removal was also performed as described above followed by washing. The resin was added to a solution of TFA/H₂O/TIPS (95%, 2.5%, 2.5%, 20 mL) with agitation for 2 h at ambient temperature. The resin was filtered, and the resulting solution was concentrated in vacuo. The residue was trituated with cold diethyl ether. The sample was analyzed for purity using a Waters 1525 Binary HPLC Pump using a Phenomenex Luna 5 u C8(2) 100 A (250×4.60 mm) column; gradient elution with H₂O/CH₃CN. Crude product was used.

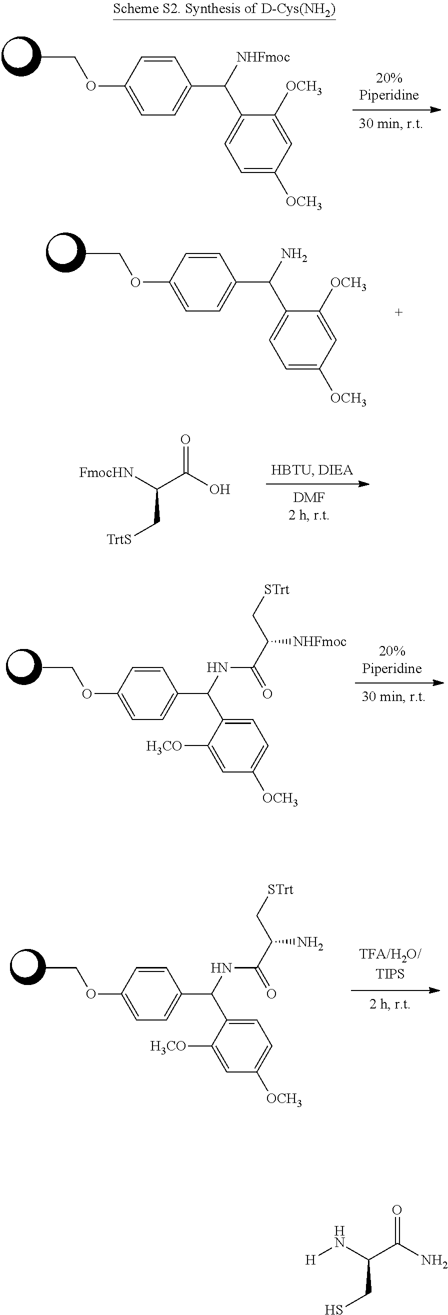

A 50 mL peptide synthesis vessel charged with rink amide resin (1000 mg, 0.45 mmol) underwent the Fmoc removal procedure and was washed as described above. Fmoc-D-Cysteine (Trt)-OH (1.5 eq, 395 mg, 0.67 mmol), HBTU (1.4 eq, 238 mg, 0.63 mmol), and DIEA (3 eq, 0.235 mL, 1.35 mmol) in DMF (20 mL) were added to the reaction vessel and agitated for 2 h at ambient temperature. After 2 h the resin was washed as previously stated and the Fmoc protecting group removal was also performed as described above followed by washing. The resin was added to a solution of TFA/H₂O/TIPS (95%, 2.5%, 2.5%, 20 mL) with agitation for 2 h at ambient temperature. The resin was filtered, and the resulting solution was concentrated in vacuo. The residue was trituated with cold diethyl ether. Crude product was used.

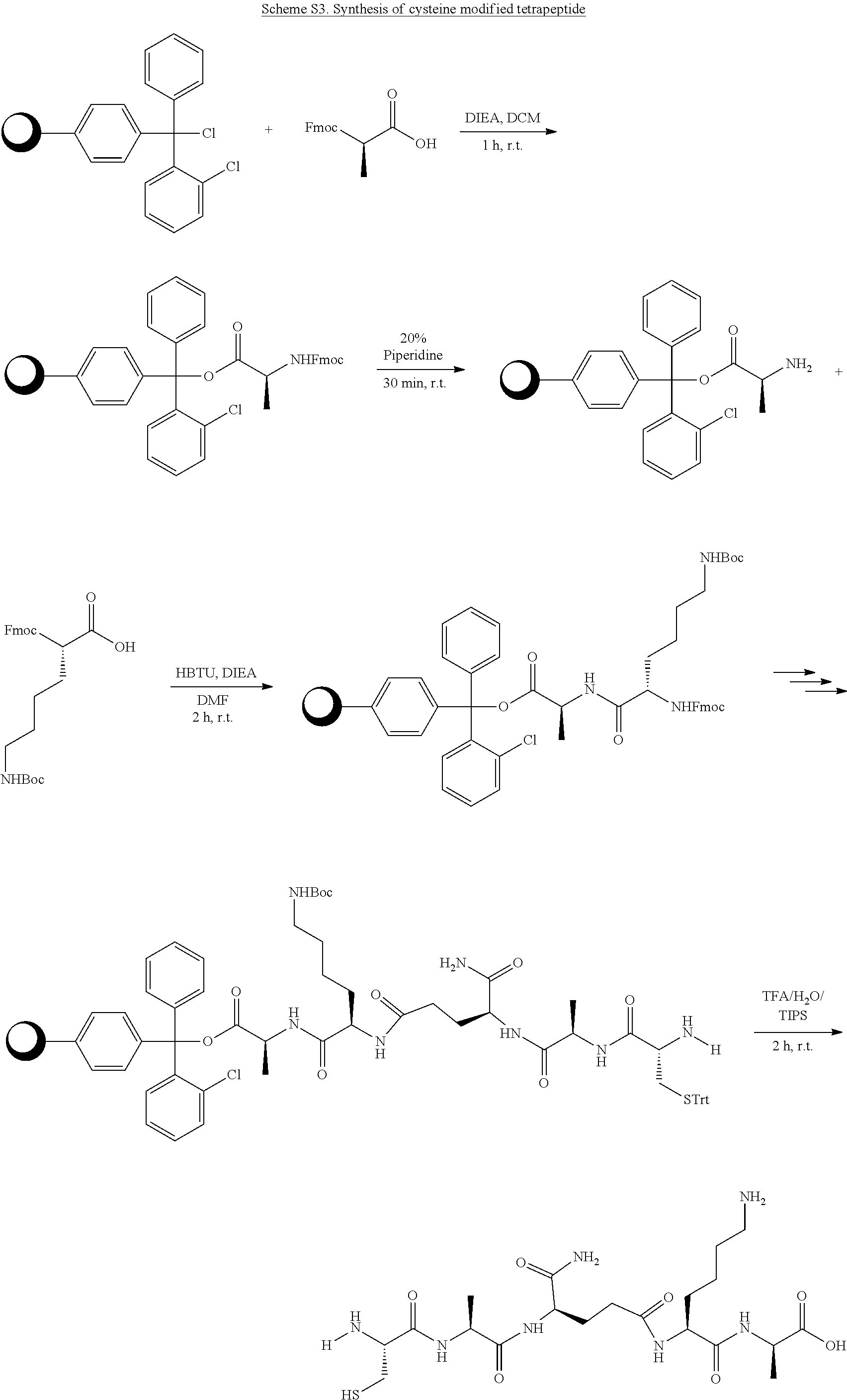

Fmoc-D-Alanine-OH (1.1 eq, 195 mg, 0.62 mmol) was added to a 25 mL peptide synthesis vessel charged with 2-Chlorotrityl chloride resin (500 mg, 0.57 mmol) and DIEA (4.4 eq, 0.436 mL, 2.50 mmol) in dry DCM (5 mL). The resin was agitated for 1 h at ambient temperature and washed as described above. The Fmoc protecting group was removed and the resin was washed as previously stated. Fmoc-L-Lysine (Boc)-OH (5 eq, 1335 mg, 2.85 mmol), HBTU (4.9 eq, 1059 mg, 2.79 mmol), and DIEA (10 eq, 0.992 mL, 5.70 mmol) in DMF (15 mL) were added to the reaction vessel and agitated for 2 h at ambient temperature. After 2 h the resin was washed as previously stated and the Fmoc protecting group removal was performed also as described above followed by washing. Fmoc-D-glutamic acid α-amide (1.5 eq, 157 mg, 0.85 mmol), HBTU (1.4 eq, 151 mg, 0.79 mmol), and DIEA (3 eq, 0.148 mL, 1.71 mmol) were added to the reaction vessel and agitated for 2 h at ambient temperature and washed as described above. The Fmoc deprotection and coupling procedure was repeated for Fmoc-L-Alanine-OH and Fmoc-L-Cysteine (Trt)-OH using the same equivalencies as used for Fmoc-L-Lysine (Boc)-OH. The Fmoc group was removed after the coupling of the last amino acid, Fmoc-L-Cysteine (Trt)-OH and washed as before. The resin was then added to a solution of TFA/H₂O/TIPS (95%, 2.5%, 2.5%, 20 mL) with agitation for 2 h at ambient temperature. The resin was filtered, and the resulting solution was concentrated in vacuo. The residue was trituated with cold diethyl ether. The compounds were purified using reverse phase HPLC using 95% H₂O/5% MeOH starting and gradient elution. The sample was analyzed for purity using a Waters 1525 Binary HPLC Pump using a Phenomenex Luna 5 u C8(2) 100 A (250×4.60 mm) column; gradient elution with H₂O/CH₃CN.

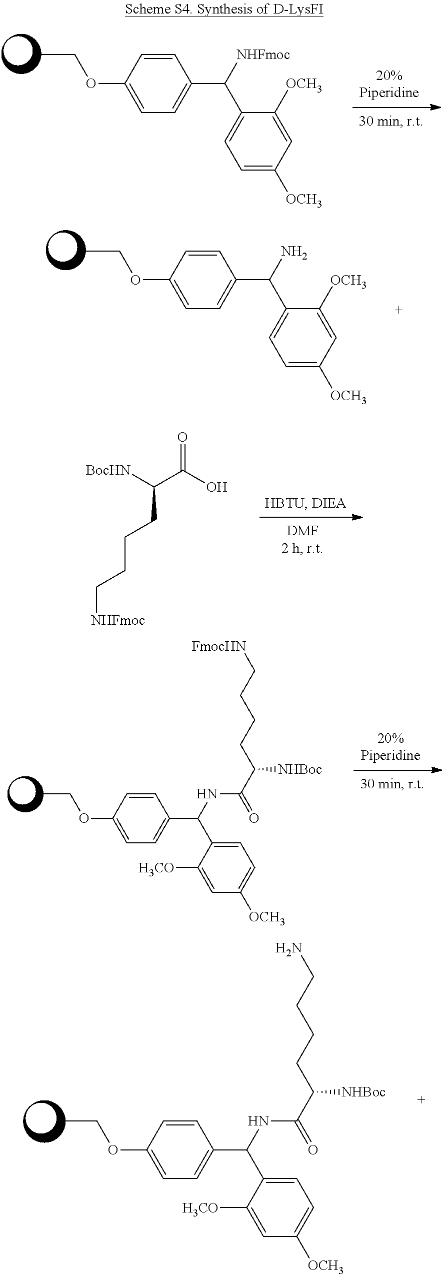

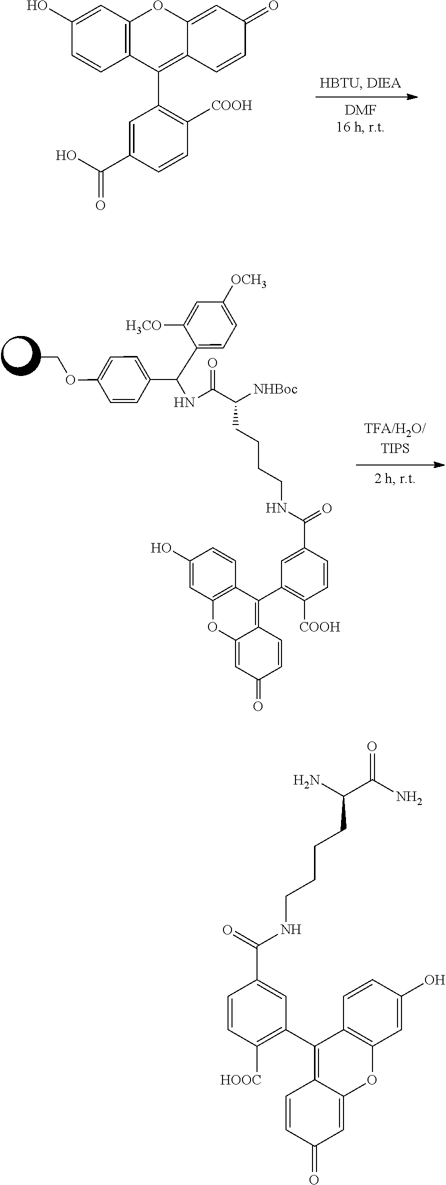 [SMILES: O=C(O)c1ccc(C(=O)O)c(-c2c3ccc(=O)cc-3oc3cc(O)ccc23)c1 ; CCOc1ccc(C(NC(=O)[C@@H](CCCCNC(=O)c2ccc(C(=O)O)c(-c3c4ccc(=O)cc-4oc4cc(O)ccc34)c2)NC(=O)OC(C)(C)C)c2ccc(OC)cc2OC)cc1 ; NC(=O)[C@H](N)CCCCNC(=O)c1ccc(C(=O)O)c(-c2c3ccc(=O)cc-3oc3cc(O)ccc23)c1]

A 25 mL peptide synthesis vessel charged with rink amide resin (250 mg, 0.11 mmol) underwent the Fmoc removal procedure and was washed as described above. Boc-D-Lysine (Fmoc)-OH (5 eq, 257 mg, 0.55 mmol), HBTU (4.9 eq, 204 mg, 0.53 mmol), and DIEA (10 eq, 0.191 mL, 1.10 mmol) in DMF (15 mL) were added to the reaction vessel and agitated for 2 h at ambient temperature. After 2 h the resin was washed as previously stated and the Fmoc protecting group removal was also performed as described above followed by washing. The resin was coupled with 5,6-carboxyfluorescein (2 eq, 82 mg, 0.22 mmol), HBTU (1.9 eq, 79 mg, 0.20 mmol), and DIEA (4 eq, 0.076 mL, 0.44 mmol) in DMF (15 mL) and agitated for 16 h at ambient temperature. The resin was washed as previously described and then added to a solution of TFA/H₂O/TIPS (95%, 2.5%, 2.5%, 20 mL) with agitation for 2 h at ambient temperature. The resin was filtered, and the resulting solution was concentrated in vacuo. The residue was trituated with cold diethyl ether. The sample was analyzed for purity using a Waters 1525 Binary HPLC Pump using a Phenomenex Luna 5 u C18(2) 100 A (250×4.60 mm) column; gradient elution with H₂O/CH₃CN. Crude product was used.

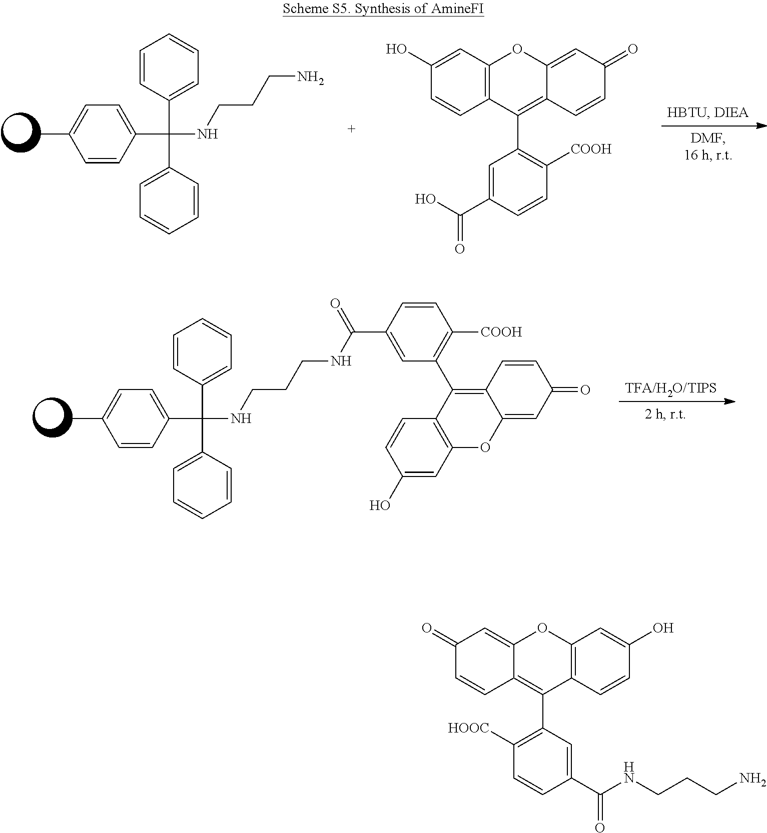 [SMILES: Cc1ccc(C(NCCCN)(c2ccccc2)c2ccccc2)cc1 ; O=C(O)c1ccc(C(=O)O)c(-c2c3ccc(=O)cc-3oc3cc(O)ccc23)c1 ; Cc1ccc(C(NCCCNC(=O)c2ccc(C(=O)O)c(-c3c4ccc(=O)cc-4oc4cc(O)ccc34)c2)(c2ccccc2)c2ccccc2)cc1 ; NCCCNC(=O)c1ccc(C(=O)O)c(-c2c3ccc(=O)cc-3oc3cc(O)ccc23)c1]

5,6-carboxyfluorescein (2 eq, 206 mg, 0.55 mmol) was added to a 25 mL peptide synthesis vessel charged with 1,3-Diaminopropane trityl resin (500 mg, 0.27 mmol), HBTU (1.9 eq, 194 mg, 0.51 mmol), and DIEA (4 eq, 0.383 mL, 1.08 mmol). The resin was agitated for 16 h at ambient temperature. Then the resin was washed as previously described and then added to a solution of TFA/H₂O/TIPS (95%, 2.5%, 2.5%, 20 mL) with agitation for 2 h at ambient temperature. The resin was filtered, and the resulting solution was concentrated in vacuo. The residue was trituated with cold diethyl ether. The resulting sample was analyzed for purity using an Agilent 1200 HPLC with a Phenomenex Luna 5μ C4 300 Å (250×2.00 mm) column; gradient elution with H₂O/CH₃CN. Crude product was used for further synthesis of PEG based library.

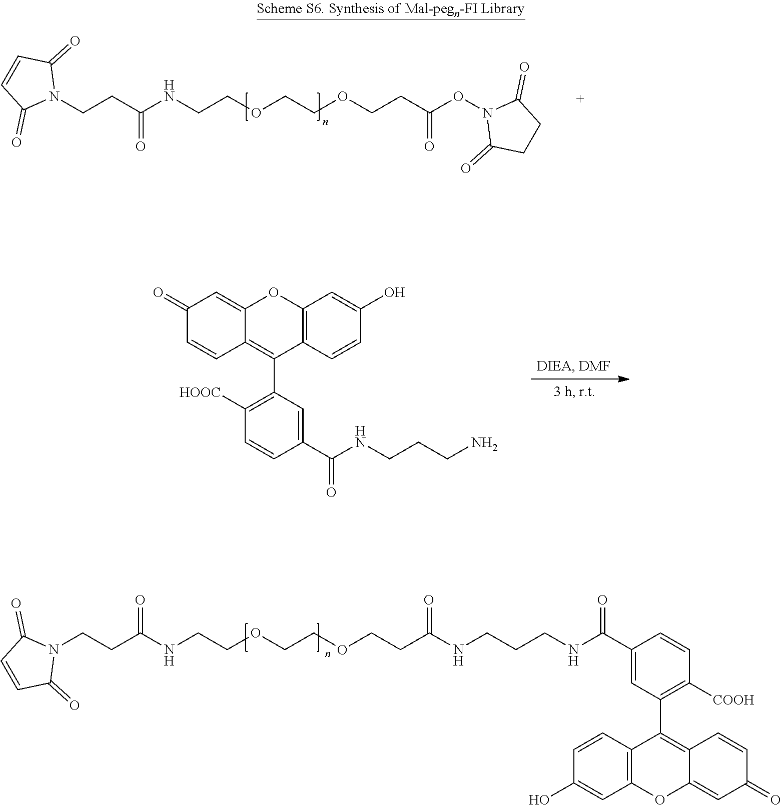

All library members were synthesized using the same method. Mal-amido-PEGₙ-NHS (n=2, 4, 6, 8, 24, 10 mg) or Mal-PEG₁₂-NHS was added to AmineFI (3 eq) dissolved in dry DMF and DIEA (3 eq). This was allowed to react for 3 h with agitation. The reaction mix was purified using reverse phase HPLC using 70% H₂O/30% MeOH starting and gradient elution. The resulting sample was analyzed for purity using an Agilent 1200 HPLC with a Phenomenex Luna 5μ C4 300 Å (250×2.00 mm) column; gradient elution with H₂O/CH₃CN.

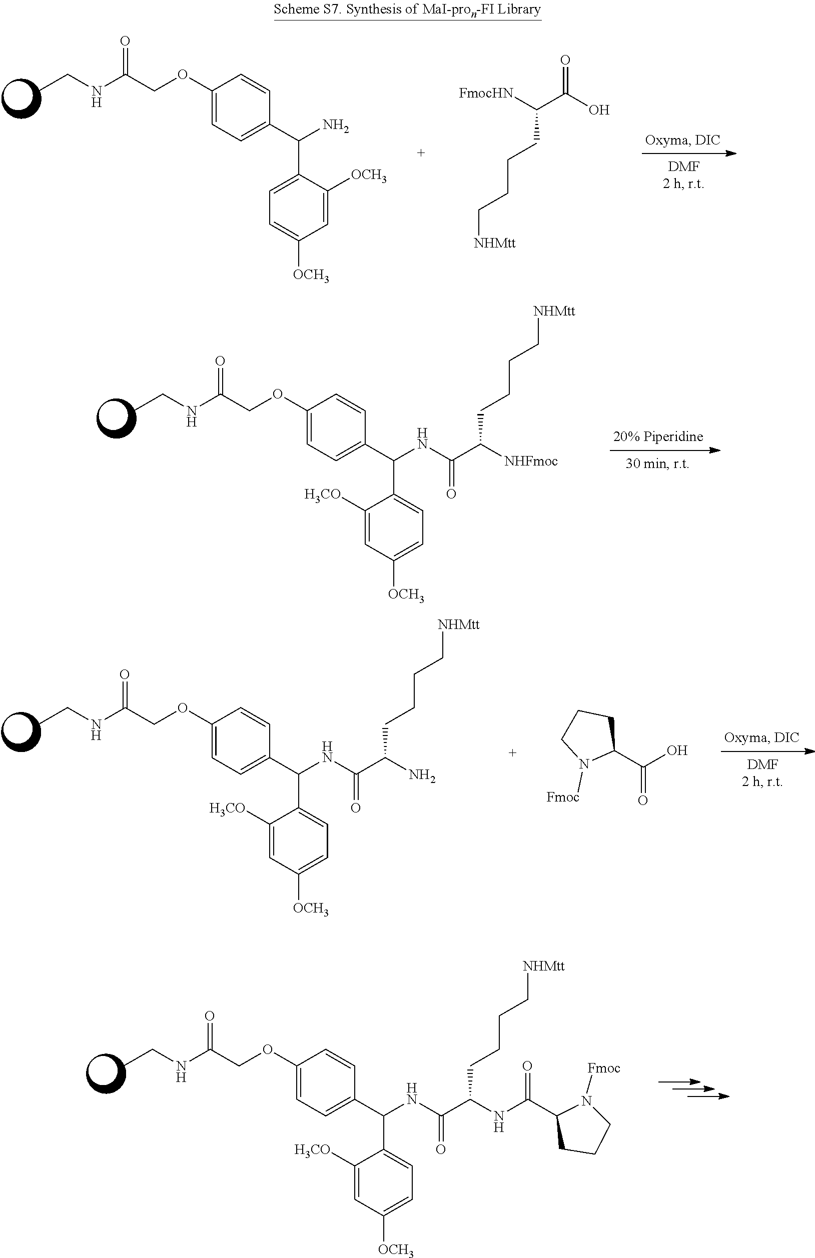

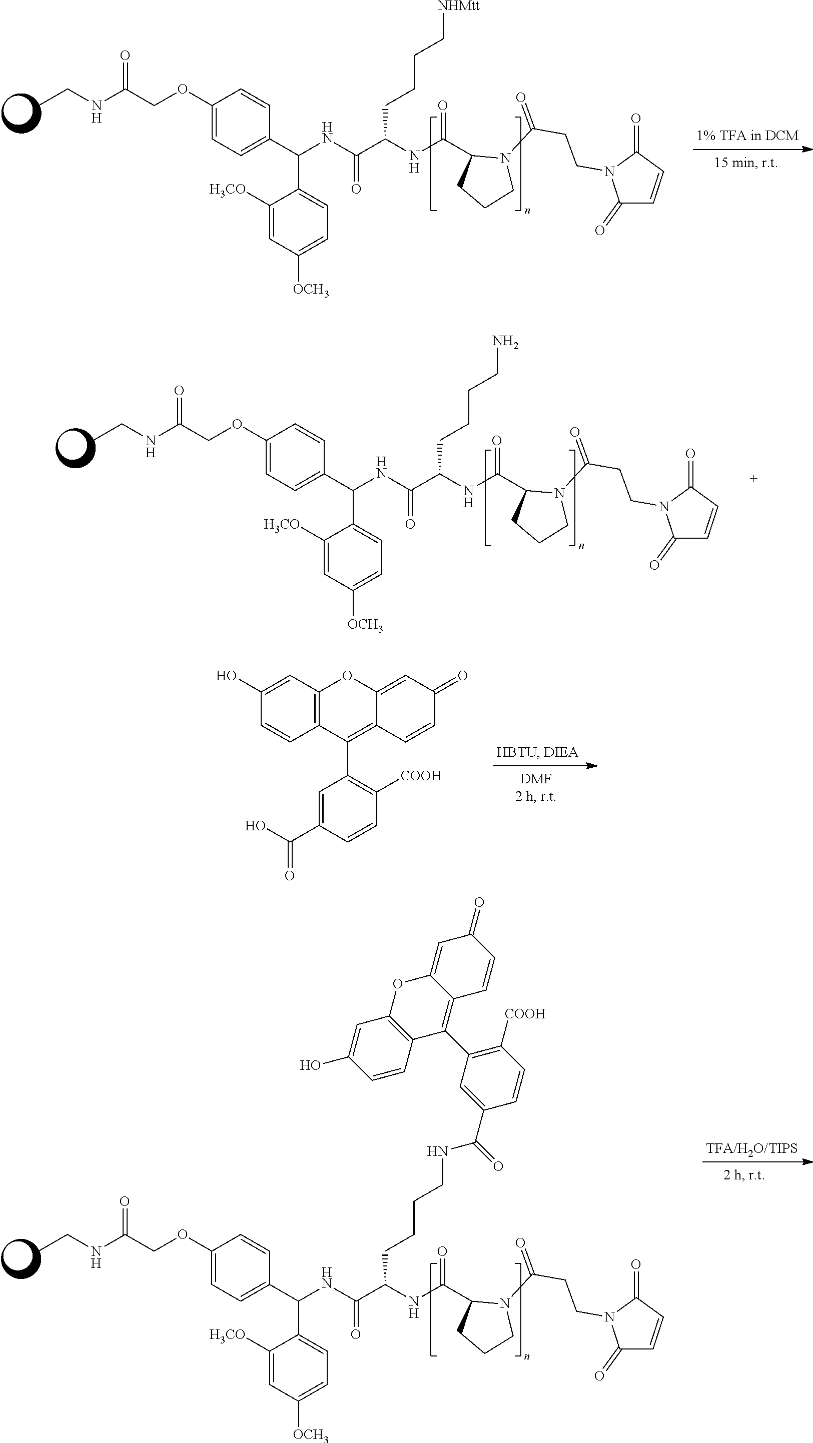

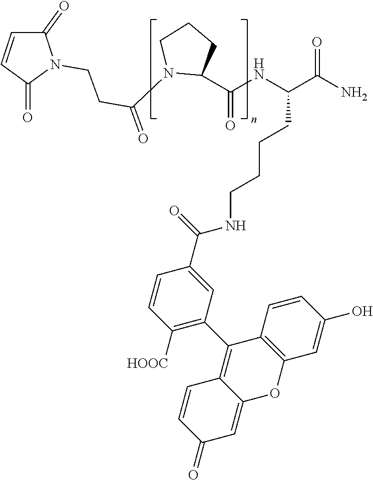

Fmoc-L-Lysine (Mtt)-OH (2 eq, 1124 mg, 1.80 mmol), Oxyma Pure (2 eq, 255 mg, 1.80 mmol), and DIC (2 eq, 0.278 mL, 1.80 mmol) were added to a 50 mL peptide synthesis vessel charged with H-Rink amide ChemMatrix resin (2000 mg, 0.90 mmol) in DMF (20 mL). The resin was agitated for 2 h at ambient temperature and washed as described above. The Fmoc protecting group was removed and the resin was washed as previously stated. Fmoc-L-Proline-OH (5 eq, 1518 mg, 4.50 mmol), Oxyma Pure (5 eq, 639 mg, 4.50 mmol), and DIC (5 eq, 0.696 mL, 4.50 mmol) in DMF (20 mL) were added to the reaction vessel and agitated for 5 minutes at ambient temperature. The resin was then drained and immediately after Fmoc-L-Proline-OH (5 eq, 1518 mg, 4.50 mmol), Oxyma Pure (5 eq, 639 mg, 4.50 mmol), and DIC (5 eq, 0.696 mL, 4.50 mmol) in DMF (20 mL) were added to the reaction vessel and that was allowed to react with agitation for 2 h at ambient temperature. After 2 h the resin was washed as previously stated. The Fmoc deprotection, previously described, and coupling procedure, at the same equivalencies, was repeated for all subsequent Fmoc-L-Proline-OH residues added to reach the desired n lengths. The resin was split off at n=3, 5, 7, 10, 13, 16, and 31 proline residues. Once the polyproline segments were built, each resin pool could undergo the Fmoc removal procedure. To the resin 3-maleimidopropionic acid (3 eq), Oxyma Pure (3 eq), and DIC (3 eq) were added and agitated for 2 h at ambient temperature. The resin was washed as described above. Next the Mtt protecting group of the lysine residue was removed by adding a TFA cocktail solution (1% TFA in DCM) to the resin and agitating for 15 minutes. The solution was drained, and this procedure was repeated five additional times. The solution was then drained, rinsed with DMF, and washed as previously described. Finally, the resin was coupled with 5,6-carboxyfluorescein (2 eq), HBTU (1.9 eq), and DIEA (4 eq) in DMF (20 mL) and agitated for 16 h at ambient temperature. The resin was washed as previously described and then added to a solution of TFA/H₂O/TIPS (95%, 2.5%, 2.5%, 20 mL) with agitation for 2 h at ambient temperature. The resin was filtered, and the resulting solution was concentrated in vacuo. The residue was trituated with cold diethyl ether. The compounds were purified using reverse phase HPLC 70% H₂O/30% MeOH starting and gradient elution. The resulting sample was analyzed for purity using an Agilent 1200 HPLC with a Phenomenex Luna 5μ C4 300 Å (250×2.00 mm) column; gradient elution with H₂O/CH₃CN.

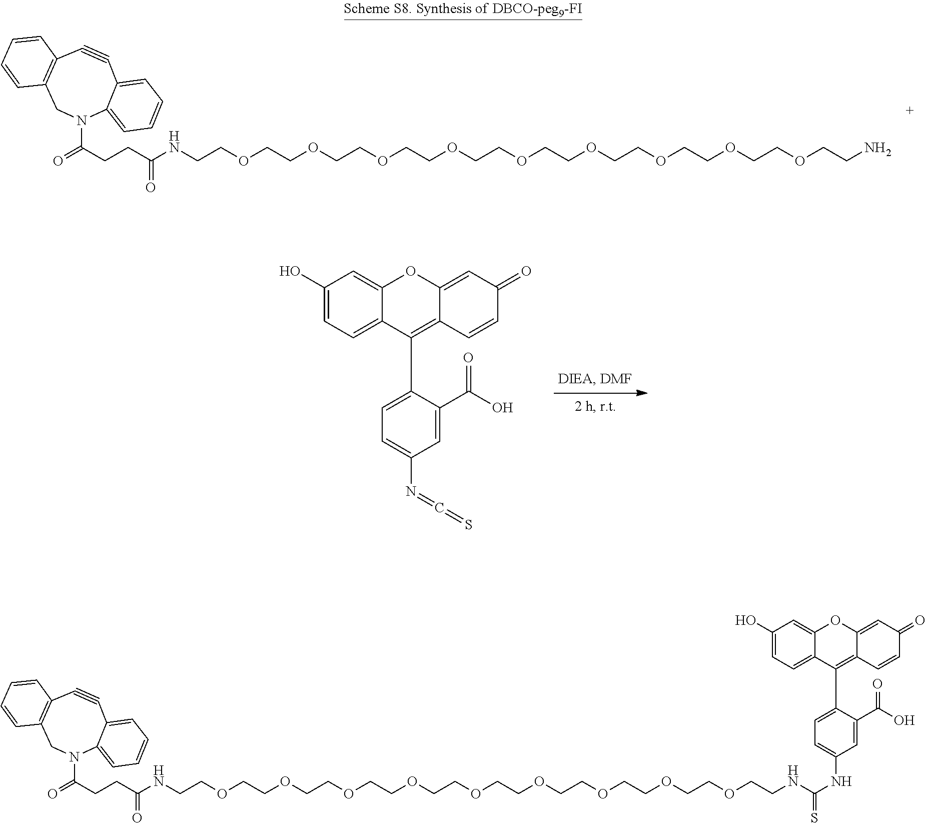 [SMILES: NCCOCCOCCOCCOCCOCCOCCOCCOCCOCCNC(=O)CCC(=O)N1Cc2ccccc2C#Cc2ccccc21 ; O=C(O)c1cc(N=C=S)ccc1-c1c2ccc(=O)cc-2oc2cc(O)ccc12 ; O=C(CCC(=O)N1Cc2ccccc2C#Cc2ccccc21)NCCOCCOCCOCCOCCOCCOCCOCCOCCOCCNC(=S)Nc1ccc(-c2c3ccc(=O)cc-3oc3cc(O)ccc23)c(C(=O)O)c1]

DBCO-PEG₉-amine was added to fluorescein isothiocyanate isomer I (2 eq) and DIEA (2 eq) in DMF and allowed to react at ambient temperature for 2 h. The reaction mix was purified using reverse phase HPLC using 70% H₂O/30% MeOH starting and gradient elution. The sample was analyzed for purity using a Waters 1525 Binary HPLC Pump using a Phenomenex Luna 5 u C8(2) 100 A (250×4.60 mm) column; gradient elution with H₂O/CH₃CN (DMSO signal has been subtracted).

### BIBLIOGRAPHY

1. Guvench, O.; Mallajosyula, S. S.; Raman, E. P.; Hatcher, E.; Vanommeslaeghe, K.; Foster, T. J.; Jamison, F. W., 2nd; Mackerell, A. D., Jr., CHARMM additive all-atom force field for carbohydrate derivatives and its utility in polysaccharide and carbohydrate-protein modeling. J Chem Theory Comput 2011, 7 (10), 3162-3180.
2. Best, R. B.; Zhu, X.; Shim, J.; Lopes, P. E.; Mittal, J.; Feig, M.; Mackerell, A. D., Jr., Optimization of the additive CHARMM all-atom protein force field targeting improved sampling of the backbone phi, psi and side-chain chi(1) and chi(2) dihedral angles. J Chem Theory Comput 2012, 8 (9), 3257-3273.
3. Vanommeslaeghe, K.; MacKerell, A. D., Jr., Automation of the CHARMM General Force Field (CGenFF) I: bond perception and atom typing. J Chem Inf Model 2012, 52 (12), 3144-54.
4. Jorgensen, W. L.; Chandrasekhar, J.; Madura, J. D.; Impey, R. W.; Klein, M. L., Comparison of Simple Potential Functions for Simulating Liquid Water. Journal of Chemical Physics 1983, 79 (2), 926-935.
5. Jo, S.; Kim, T.; Iyer, V. G.; Im, W., CHARMM-GUI: a web-based graphical user interface for CHARMM. J Comput Chem 2008, 29 (11), 1859-65.
6. Eastman, P.; Swails, J.; Chodera, J. D.; McGibbon, R. T.; Zhao, Y.; Beauchamp, K. A.; Wang, L. P.; Simmonett, A. C.; Harrigan, M. P.; Stern, C. D.; Wiewiora, R. P.; Brooks, B. R.; Pande, V. S., OpenMM 7: Rapid development of high performance algorithms for molecular dynamics. PLoS Comput Biol 2017, 13 (7), e1005659.
7. Lee, J.; Cheng, X.; Jo, S.; MacKerell, A. D.; Klauda, J. B.; Im, W., CHARMM-GUI Input Generator for NAMD, Gromacs, Amber, Openmm, and CHARMM/OpenMM Simulations using the CHARMM36 Additive Force Field. Biophys J 2016, 110 (3), 641a-641a.

### Example III

Small Molecule Permeation Across Mycomembrane of Live Cells. Similar to Gram-negative bacteria, mycobacteria possess an outer membrane (OM) that encases the entire cell.^{1, 2} The double membrane mycomembrane serves as a formidable barrier that is thought to hinder the penetration of small molecules. As such, it has been implicated in endowing mycobacteria with a high level of intrinsic drug resistance to antimycobacterial agents.³ Given is location—lying at the interface between the potentially vulnerable inner components—and the host, the mycomembrane is central to the host-mycobacteria relationship. Lack of permeation across the mycomembrane has long been hypothesized to be one of the primary reasons for the failure of antibiotics in mycobacteria. While there is some variability across various mycobacterial species, there are several conserved components within the cellular envelope. The outer leaflet is composed primarily of polyacyltrahalose (PAT), diacyltreahalose (DAT), and trehalose dimycolate (TDM) molecules. Within the inner leafleft of the mycomembrane, there are a number of noncovalently linked lipids and lipoglycans whose alkyl chains can range from C₆₀ to C₉₀. Aside from the mycomembrane itself, the heteropolysaccharide arabinogalactan layer, which is covalently attached to the mycolic acids, can also act as a permeation barrier.

### Click Chemistry-Based Accessibility to the PG.

The therapeutic effectiveness of most antimycobacterial agents is dependent on their ability to permeate through the mycomembrane to ultimately reach their cellular target. To this end, most antimycobaterial agents are relatively small and hydrophobic, in contrast to other known antibiotics. The following are structures of small molecule anti-TB agents:

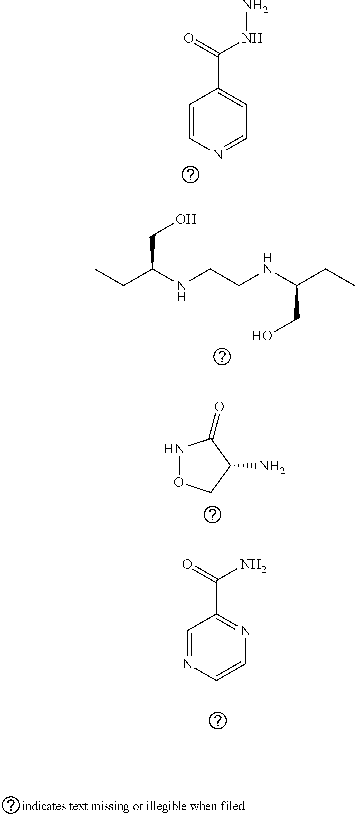

Antimycobacterial drugs that are larger have been proposed to cross across a porin (e.g., MspA in M. smegmatis).⁴ We reasoned that we could quantitatively probe the permeation of small molecules across the mycomembrane by measuring the quantity of molecules that reach the peptidoglycan (PG) scaffold.

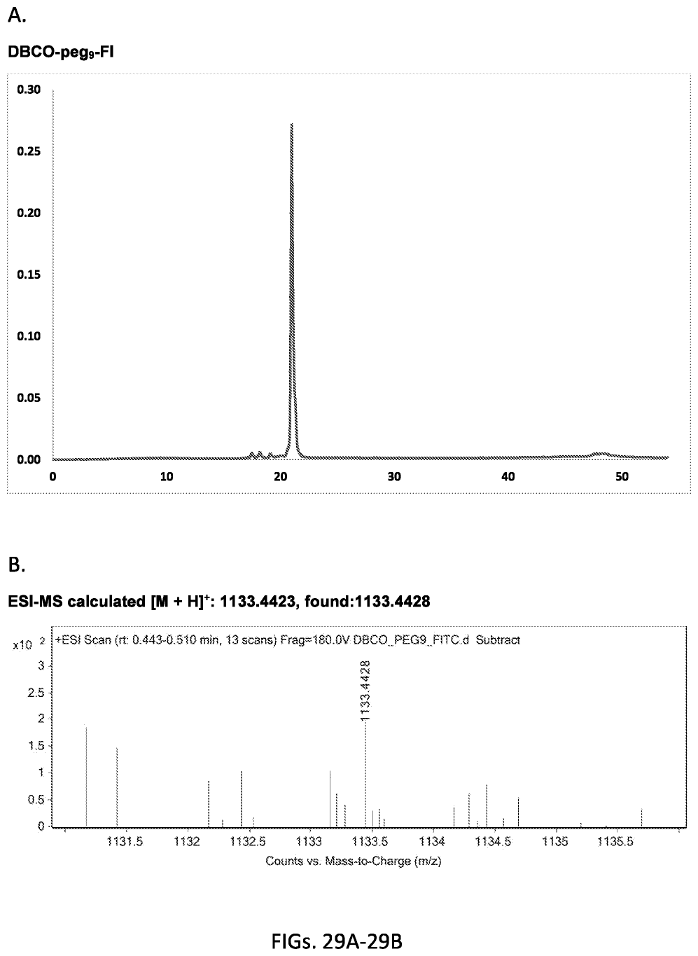

Mycobacteria have a cell envelope that includes a PG layer within the periplasmic space on exterior side of the cytoplasmic membrane (FIG. 29A). PG is a mesh-like polymer made up of repeating disaccharides N-acetylglucosamine (GlcNAc) and N-acetylmuramic acid (MurNAc) (FIG. 29B). Each MurNAc unit is connected to a short and unusual peptide (stem peptide) with the canonical sequence of L-Ala-D-iGlu-m-DAP-D-Ala-D-Ala.⁵ In mycobacteria, neighboring stem peptides are crosslinked by transpeptidases to endow the PG matrix with high levels of rigidity and integrity. Bacterial PG is an essential component of the bacterial cell wall, which makes it an attractive target for the design of antimycobacterial agents. Critically, a large number of FDA-approved antibiotics work by inhibiting biosynthesis of PG and permeation to various steps within PG biosynthesis must also traverse the mycomembrane.^{6, 7}

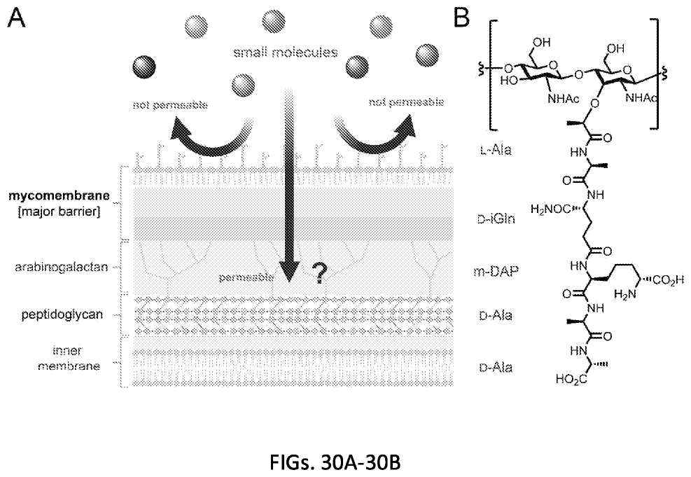

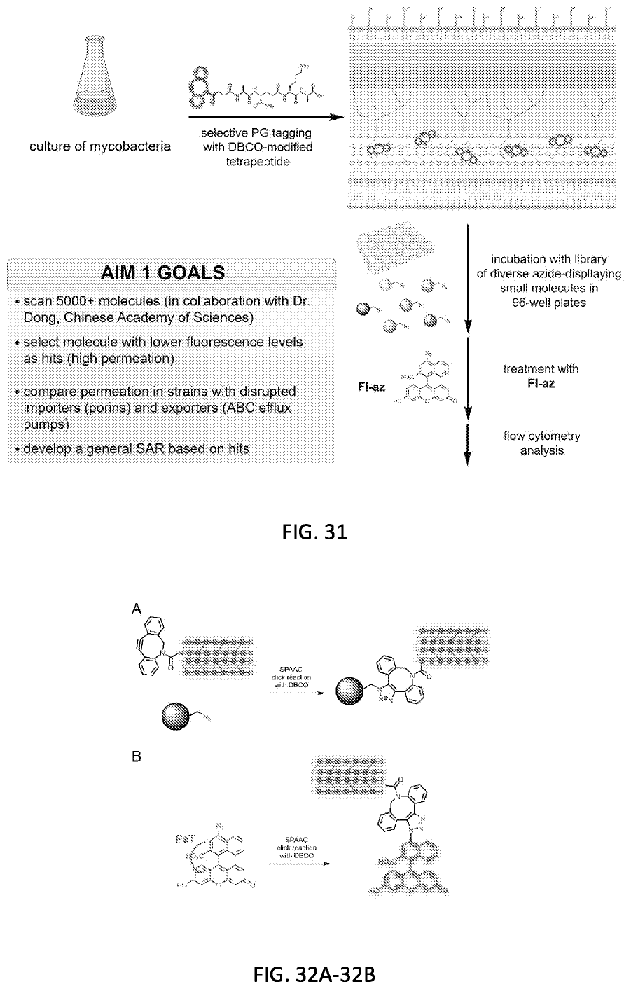

Competition Assay Design. We envisioned that the site selective metabolic installation of a biorthogonal (“click”) handle within the PG scaffold of mycobacteria could be leveraged to assess the accumulation of small molecules beyond the outer mycomembrane (FIG. 30). This novel workflow results in a Peptidoglycan Accessibility Click Assay (PACA). First, mycobacterial cells are treated with PG metabolic tags overnight to afford cells with an even distribution of the click handle within the entire PG scaffold. After washing the cells to remove the excess metabolic tags from the media, cells will then be treated with small molecules from a large and diverse library; each molecule will contain a complementary click handle in a 96-well format. For molecules that permeate past the mycomembrane, they are expected to covalently react with PG scaffold via the click reaction. More specifically, the PG metabolic tag will be conjugated to a DiBenzoCycloOctyne (DBCO) handle, which will lead to the display of DBCO epitopes within the PG scaffold.^{8, 9} Part of the design of this assay, the small molecules will each include an azide handle that is small and minimally perturbs the physiochemical properties of the small molecules. This pair of reactive functional groups is biorthogonal and readily forms a triazole covalent bond in the absence of metal catalysts based on strain promoted alkyne-azide cycloaddition (SPAAC).¹⁰ After the azide-containing small molecules are incubated with the mycobacterial cells, cells are washed and treated with internally quenched azido-containing fluorescein.¹¹ The aryl azide group within fluorescein results in a photoinduced electron transfer (PeT) in the azide form, which unquenches upon the reaction of DBCO imbedded in the PG leading to a large turn-on in fluorescence levels (FIG. 31A-B). Turn-on fluorescence mechanism is intended to reduce the background signal of fluorophores not PG-bound and it can potentially shift the assay from flow cytyometry-based analysis to multi-channel fluorometer. Finally, cells are analyzed by flow cytometry using a 96-well plate format. Small molecules that result in a decrease in cellular fluorescence are suggestive of structural motifs that favor high levels of permutability across the mycomembrane.

Screening Program. By screening a structurally diverse library using a high density 96-well plate format, an unprecedented level of insight into the physiochemical properties that modulate small molecule permeation across mycomembranes is provided. These results can be mined in the future to formulate predictive rules that can guide drug discovery programs for molecules that show high levels of permeation. Similar attempts to establish insight into penetration across the OM of E. coli, although at a significantly smaller scale at 180 molecules, was recently described using a low-throughput method of liquid chromatography and mass spectrometry (Nature, 2017 545: 299-304).¹² Despite the lower capacity of this method, even 180 molecules was sufficiently informative to yield novel insight into the physiochemical features that result in high OM permeation. In contrast, the instant assay is facile (only requiring reagents that are commonplace to the PIs and standard instrumentation), compatible with diverse types of bacteria (no need for genetic manipulation) including Mtb and their drug-resistant strains, and readily scalable to high-throughput screening platforms. The library of diverse azide-containing molecules can be obtained commercially or generated (e.g., recently described modular method to convert primary amines to azides (Dr. Jiajia Dong and Dr. Barry Sharpless, Nature 2019 574:86-89; the PIs originally demonstrated that their method could be applied to assemble a 1,200-member azide library on 96-well plates. They have since improved on this throughput and have a 5,000-member library, which is fully assembled).

### Data

The basis of the assay is a site selective incorporation of a reactive biorthogonal epitope within the PG of live cells; cellular assays that report on accessibility of molecules to the surface of bacterial cells. We recently described a live cell fluorescence assay that reports on the accessibility of molecules to and within the PG scaffold in Staphylococcus aureus. The study showed that teichoic acids impede the permeability of molecules of a wide range of sizes and chemical composition. Gram-positive bacteria, such as S. aureus, lack an outer membrane, and, therefore, this prior work focused on the ability of molecules to access the extracellular PG. Nonetheless, there are fundamental aspects of the assay that are adopting to establish mycomembrane permeation. Covalent PG tagging is expected to result in reliable measurements that can be readily quantified using standard techniques amendable to high throughput analyses (e.g., flow cytometry). We initially reasoned that the DBCO unit could be linked to a single D-amino acid to promote metabolic remodeling of the bacterial PG. We synthesized both D-lysine and D-2,4-diaminobutyric acid (Dab) functionalized with DBCO on the sidechain amino group. During cell growth, exogenously single D-amino acids supplemented in the culture medium are swapped in the place of the D-alanine that occupies the 4^{th} or 5^{th} position within the stem peptide. A wide range of single D-amino acid PG probes have been developed to elucidate fundamental steps in bacterial cell wall biology.^{1, 13-28}

After overnight treatment of Mycobacterium smegmatis (M. smegmatis) with either DBCO-modified amino acid or vehicle, cells were subsequently treated with azide-modified fluorescein. As a control, cells were also treated with azide-modified D-lysine, D-LysAz, and fluorescently labeled with DBCO-modified fluorescein. We previously showed that the treatment of M. smegmatis with D-LysAz affords cells labeled with azide groups within the PG scaffold. Surprisingly, there was no labeling of mycobacterial cells with either Lys or Dab modified with DBCO. As expected, the positive control conditions led to high levels of cellular fluorescence thus indicating the SPAAC is operative within M. smegmatis. While it remains unclear why the conjugation of the DBCO onto the sidechain of a D-amino acid resulted in minimal PG labeling, we posed that that the large size of DBCO played a role. These results prompted us to alter the design of the PG labeling. Instead of using a single amino acid, we conjugated DBCO onto the N-terminus of a tetrapeptide synthetic analog of the PG stem peptide. We^{23, 29} and others³⁰⁻³³, recently showed that structural analogs of PG stem peptides can be crosslinked into the growing PG scaffold of live cells. We had previously found that the N-terminus of the tetrapeptide is much more tolerant to conjugates than the single D-amino acids due to the pathway that it hijacks for PG incorporation. When we tested the labeling of M. smegmatis with the DBCO-tetrapeptide, it was observed that cellular fluorescence levels were high, which are suggestive of efficient DBCO tagging within the PG scaffold. Further confirmation of the location of the PG tag was performed using LC-MS analysis and confocal microscopy with the isolated sacculi (data not shown).

Validation of permeation assay. Next, we set out to benchmark the ability of the assay to report on the permeability of a test small molecule. For this assay, L-LysN₃ was chosen as the test molecule. As before, the PG of live M. smegmatis was metabolically tagged with DBCO. After a washing step, cells were treated with increasing concentrations of the test compound 3-azido-1-propanamine. After removal of excess molecules, cells were treated with azide-modified fluorescein, washed with PBS, fixed with 4% formaldehyde in PBS, and analyzed by flow cytometry. At lower concentrations, the cellular fluorescence levels were higher, presumably because fewer reactions with the test molecule occurred, thus leaving free DBCO epitopes to react with fluorescein. Titrating of increasing levels of the test molecule led to a progressive decrease in cellular fluorescence, which is consistent with increasing permeability of the test molecule across the mycomembrane. Additional experiments were performed to optimize the concentration of the PG metabolic tag, the incubation period with the small molecules, and the concentration of the azide-fluorescein (data not shown). With the assay conditions defined, we then set out to perform a pilot screen that included 10 azide-conjugated test compounds. The pilot screen was performed to demonstrate the robustness of the assay in a 96-well plate format and its reproducibility. We found that the assay performed extremely well, demonstrating a significant dynamic range in signal and excellent inter-assay reproducibility.

The preliminary results can be expanded to perform a large-scale screen that will include up to 5,000 azide-modified small molecules, which can be accomplished, for example, using either 96-well and/or 384-well plate formats on the Attune Flow Cytometer (ThermoFisher, Inc.). For this phase of the work, the screening program will focus on M. smegmatis as a model organism for mycobacteria. Briefly, the DBCO-tetrapeptide will be synthesized as described above. All peptides will be purified using standard RP-HPLC and their purities will be verified using a combination of analytical HPLC and the identity will be confirmed using HR-MS and NMR. To start the screen, a large (100 mL to screen 1,000 compounds at a time) culture will be started by inoculating the medium at 1:100 in the presence of DBCO-tetrapeptide (100 μM). After overnight incubation, cells will be washed with PBS three-times and dispensed into ten 96-well microtiter plates. Individual azide-conjugated small molecules (50 μM) will be subsequently added to the cells to interrogate their permeation past the mycomembrane at 50 μM for 1 hour. Following this step, the cells will once again be washed, treated with azide-conjugated fluorescein (25 μM for 30 min), washed with PBS, fixed with 4% formaldehyde in PBS, then analyzed by flow cytometry. To test the assay reproducibility, 3 of the plates (out of the fifty-two 96-well plates) will be re-analyzed using identical conditions and considered to be satisfactory is there is less than five percent deviation across the two runs. Using these results, the library will be analyzed for structural motifs that result in greater permeability. Scientific Rigor: Measurements of labeling will be performed in two biological replicates. Bacteria labeling levels will be compared against bacteria treated with no D-LysAz or azide-fluorescein alone through multiple comparisons analyses using either the Dunnett (at 95% confidence intervals) or the Holm-Ŝidák tests (GraphPad for PC).

Other types of BSL2 mycobacteria will also be analyzed in a similar manner, including Mycobacteria marinum, Mycobacterium avium, and BCG. A similar workflow will also be applied to Mtb, including clinical isolates. The assay will be carried out in accordance with strict BSL3 containment protocols and procedures. Following fixation, the samples will be safe to be transported to the room housing the flow cytometer for fluorescence analysis.

Differential Screen for Porin-mediated Permeation and Efflux Pump Recognition. The barrier properties of outer membranes can be overcome by the passive permeation via porin imbedded within the lipid layer. As a prominent example in Gram-negative bacteria, several antibiotics have been demonstrated to promote the accumulation of molecules past the OM and into the periplasm.³⁴ A porin (MspA) has been previously identified in fast-growing M. smegmatis.⁴ Moreover, the expression of MspA in Mtb was found to significantly sensitize them to antibiotics.³⁵ It was also demonstrated that hydrophilic antibiotics such as norfloxacin and chloramphenicol diffuse past the mycomembrane through the MspA porin in M. smegmatis.³⁶ In Mtb, it is less clear what porin-like proteins may exist and the extent to which they pass facilitate the permeability of small molecules such as antibiotics. Last year, the proline-proline-glutamate (PPE) family proteins were found to facilitate small molecule permeation analogous to outer membrane porins (Science 2020, 367, 1147).³⁷ Mutations within PE/PPE, which was shown to reside within the outer membrane of Mtb, resulted in resistance to the antimycobacterial agent 3bMP1. It remains poorly described the types of structural motifs that are permissive for permeation cross these porins. We propose to perform a differential screen in M. smegmatis across WT and □mspa using the entire 5,000-member azide library, which will provide an extensive molecular map of MspA-recognition. Moreover, an analogous screen will be performed in Mtb with disruptive mutations in PPE.

### BIBLIOGRAPHY

1. Siegrist, M. S., Swarts, B. M., Fox, D. M., Lim, S. A., and Bertozzi, C. R. (2015) Illumination of growth, division and secretion by metabolic labeling of the bacterial cell surface, FEMS Microbiol Rev 39, 184-202.
2. Dulberger, C. L., Rubin, E. J., and Boutte, C. C. (2020) The mycobacterial cell envelope—a moving target, Nat Rev Microbiol 18, 47-59.
3. Bansal-Mutalik, R., and Nikaido, H. (2014) Mycobacterial outer membrane is a lipid bilayer and the inner membrane is unusually rich in diacyl phosphatidylinositol dimannosides, Proc Natl Acad Sci USA 111, 4958-4963.
4. Faller, M., Niederweis, M., and Schulz, G. E. (2004) The structure of a mycobacterial outer-membrane channel, Science 303, 1189-1192.
5. Egan, A. J. F., Errington, J., and Vollmer, W. (2020) Regulation of peptidoglycan synthesis and remodelling, Nat Rev Microbiol 18, 446-460.
6. Royet, J., and Dziarski, R. (2007) Peptidoglycan recognition proteins: pleiotropic sensors and effectors of antimicrobial defences, Nat Rev Microbiol 5, 264-277.
7. Wolf, A. J., and Underhill, D. M. (2018) Peptidoglycan recognition by the innate immune system, Nat Rev Immunol 18, 243-254.
8. Baskin, J. M., Prescher, J. A., Laughlin, S. T., Agard, N. J., Chang, P. V., Miller, I. A., Lo, A., Codelli, J. A., and Bertozzi, C. R. (2007) Copper-free click chemistry for dynamic in vivo imaging, Proc Natl Acad Sci USA 104, 16793-16797.
9. Jewett, J. C., Sletten, E. M., and Bertozzi, C. R. (2010) Rapid Cu-free click chemistry with readily synthesized biarylazacyclooctynones, J Am Chem Soc 132, 3688-3690.
10. Agard, N. J., Prescher, J. A., and Bertozzi, C. R. (2004) A strain-promoted [3+2] azide-alkyne cycloaddition for covalent modification of biomolecules in living systems, J Am Chem Soc 126, 15046-15047.
11. Shieh, P., Dien, V. T., Beahm, B. J., Castellano, J. M., Wyss-Coray, T., and Bertozzi, C. R. (2015) CalFluors: A Universal Motif for Fluorogenic Azide Probes across the Visible Spectrum, J Am Chem Soc 137, 7145-7151.
12. Richter, M. F., Drown, B. S., Riley, A. P., Garcia, A., Shirai, T., Svec, R. L., and Hergenrother, P. J. (2017) Predictive compound accumulation rules yield a broad-spectrum antibiotic, Nature 545, 299-304.
13. Kuru, E., Hughes, H. V., Brown, P. J., Hall, E., Tekkam, S., Cava, F., de Pedro, M. A., Brun, Y. V., and VanNieuwenhze, M. S. (2012) In Situ probing of newly synthesized peptidoglycan in live bacteria with fluorescent D-amino acids, Angew Chem Int Ed Engl 51, 12519-12523.
14. Kuru, E., Tekkam, S., Hall, E., Brun, Y. V., and Van Nieuwenhze, M. S. (2015) Synthesis of fluorescent D-amino acids and their use for probing peptidoglycan synthesis and bacterial growth in situ, Nat Protoc 10, 33-52.
15. Siegrist, M. S., Whiteside, S., Jewett, J. C., Aditham, A., Cava, F., and Bertozzi, C. R. (2013) (D)-Amino acid chemical reporters reveal peptidoglycan dynamics of an intracellular pathogen, ACS Chem Biol 8, 500-505.
16. Lebar, M. D., May, J. M., Meeske, A. J., Leiman, S. A., Lupoli, T. J., Tsukamoto, H., Losick, R., Rudner, D. Z., Walker, S., and Kahne, D. (2014) Reconstitution of peptidoglycan cross-linking leads to improved fluorescent probes of cell wall synthesis, J Am Chem Soc 136, 10874-10877.
17. Lupoli, T. J., Tsukamoto, H., Doud, E. H., Wang, T. S., Walker, S., and Kahne, D. (2011) Transpeptidase-mediated incorporation of D-amino acids into bacterial peptidoglycan, J Am Chem Soc 133, 10748-10751.
18. Fura, J. M., Kearns, D., and Pires, M. M. (2015) D-Amino Acid Probes for Penicillin Binding Protein-based Bacterial Surface Labeling, J Biol Chem 290, 30540-30550.
19. Fura, J. M., Sabulski, M. J., and Pires, M. M. (2014) D-amino acid mediated recruitment of endogenous antibodies to bacterial surfaces, ACS Chem Biol 9, 1480-1489.
20. Pidgeon, S. E., and Pires, M. M. (2017) Cell Wall Remodeling of Staphylococcus aureus in Live Caenorhabditis elegans, Bioconjug Chem 28, 2310-2315.
21. Pidgeon, S. E., Fura, J. M., Leon, W., Birabaharan, M., Vezenov, D., and Pires, M. M. (2015) Metabolic Profiling of Bacteria by Unnatural C-terminated D-Amino Acids, Angew Chem Int Ed Engl 54, 6158-6162.
22. Apostolos, A. J., Pidgeon, S. E., and Pires, M. M. (2020) Remodeling of Cross-bridges Controls Peptidoglycan Cross-linking Levels in Bacterial Cell Walls, ACS Chem Biol 15, 1261-1267.
23. Pidgeon, S. E., Apostolos, A. J., Nelson, J. M., Shaku, M., Rimal, B., Islam, M. N., Crick, D. C., Kim, S. J., Pavelka, M. S., Kana, B. D., and Pires, M. M. (2019) L,D-Transpeptidase Specific Probe Reveals Spatial Activity of Peptidoglycan Cross-Linking, ACS Chem Biol 14, 2185-2196.
24. Fura, J. M., Pidgeon, S. E., Birabaharan, M., and Pires, M. M. (2016) Dipeptide-Based Metabolic Labeling of Bacterial Cells for Endogenous Antibody Recruitment, ACS Infect Dis 2, 302-309.
25. Caparros, M., Aran, V., and de Pedro, M. A. (1992) Incorporation of S-[3H]methyl-D-cysteine into the peptidoglycan of ether-treated cells of Escherichia coli, FEMS Microbiol Lett 72, 139-146.
26. Cava, F., Lam, H., de Pedro, M. A., and Waldor, M. K. (2011) Emerging knowledge of regulatory roles of D-amino acids in bacteria, Cell Mol Life Sci 68, 817-831.
27. de Pedro, M. A., Quintela, J. C., Holtje, J. V., and Schwarz, H. (1997) Murein segregation in Escherichia coli, J Bacteriol 179, 2823-2834.
28. Schouten, J. A., Bagga, S., Lloyd, A. J., de Pascale, G., Dowson, C. G., Roper, D. I., and Bugg, T. D. (2006) Fluorescent reagents for in vitro studies of lipid-linked steps of bacterial peptidoglycan biosynthesis: derivatives of UDPMurNAc-pentapeptide containing d-cysteine at position 4 or 5, Mol Biosyst 2, 484-491.
29. Apostolos, A. J., Pidgeon, S. E., and Pires, M. M. (2020) Remodeling of Crossbridges Controls Peptidoglycan Crosslinking Levels in Bacterial Cell Walls, ACS Chem Biol.
30. Welsh, M. A., Taguchi, A., Schaefer, K., Van Tyne, D., Lebreton, F., Gilmore, M. S., Kahne, D., and Walker, S. (2017) Identification of a Functionally Unique Family of Penicillin-Binding Proteins, J Am Chem Soc 139, 17727-17730.
31. Gautam, S., Kim, T., Shoda, T., Sen, S., Deep, D., Luthra, R., Ferreira, M. T., Pinho, M. G., and Spiegel, D. A. (2015) An Activity-Based Probe for Studying Crosslinking in Live Bacteria, Angew Chem Int Ed Engl 54, 10492-10496.
32. Gautam, S., Kim, T., and Spiegel, D. A. (2015) Chemical probes reveal an extraseptal mode of cross-linking in Staphylococcus aureus, J Am Chem Soc 137, 7441-7447.
33. Ngadjeua, F., Braud, E., Saidjalolov, S., Iannazzo, L., Schnappinger, D., Ehrt, S., Hugonnet, J. E., Mengin-Lecreulx, D., Patin, D., Etheve-Quelquejeu, M., Fonvielle, M., and Arthur, M. (2018) Critical Impact of Peptidoglycan Precursor Amidation on the Activity of l,d-Transpeptidases from Enterococcus faecium and Mycobacterium tuberculosis, Chemistry.
34. Prajapati, J. D., Kleinekathofer, U., and Winterhalter, M. (2021) How to Enter a Bacterium: Bacterial Porins and the Permeation of Antibiotics, Chem Rev 121, 5158-5192.
35. Lamrabet, O., Ghigo, E., Mege, J. L., Lepidi, H., Nappez, C., Raoult, D., and Drancourt, M. (2014) MspA-Mycobacterium tuberculosis-transformant with reduced virulence: the “unbirthday paradigm”, Microb Pathog 76, 10-18.
36. Danilchanka, O., Pavlenok, M., and Niederweis, M. (2008) Role of porins for uptake of antibiotics by Mycobacterium smegmatis, Antimicrob Agents Chemother 52, 3127-3134.
37. Wang, Q., Boshoff, H. I. M., Harrison, J. R., Ray, P. C., Green, S. R., Wyatt, P. G., and Barry, C. E., 3rd. (2020) PE/PPE proteins mediate nutrient transport across the outer membrane of Mycobacterium tuberculosis, Science 367, 1147-1151.
38. De Rossi, E., Ainsa, J. A., and Riccardi, G. (2006) Role of mycobacterial efflux transporters in drug resistance: an unresolved question, FEMS Microbiol Rev 30, 36-52.
39. Du, D., Wang-Kan, X., Neuberger, A., van Veen, H. W., Pos, K. M., Piddock, L. J. V., and Luisi, B. F. (2018) Multidrug efflux pumps: structure, function and regulation, Nat Rev Microbiol 16, 523-539.
40. Braibant, M., Gilot, P., and Content, J. (2000) The ATP binding cassette (ABC) transport systems of Mycobacterium tuberculosis, FEMS Microbiol Rev 24, 449-467.
41. Dulyayangkul, P., Calvopina, K., Heesom, K. J., and Avison, M. B. (2020) Novel Mechanisms of Efflux-Mediated Levofloxacin Resistance and Reduced Amikacin Susceptibility in Stenotrophomonas maltophilia, Antimicrob Agents Chemother 65.
42. Hrycyna, C. A., and Gottesman, M. M. (1998) Multidrug ABC transporters from bacteria to man: an emerging hypothesis for the universality of molecular mechanism and function, Drug Resist Updat 1, 81-83.
43. Hulen, C., Racine, P. J., Chevalier, S., Feuilloley, M., and Lomri, N. E. (2020) Identification of the PA1113 Gene Product as an ABC Transporter Involved in the Uptake of Carbenicillin in Pseudomonas aeruginosa PAO1, Antibiotics (Basel) 9.
44. Lubelski, J., Konings, W. N., and Driessen, A. J. (2007) Distribution and physiology of ABC-type transporters contributing to multidrug resistance in bacteria, Microbiol Mol Biol Rev 71, 463-476.
45. Orelle, C., Mathieu, K., and Jault, J. M. (2019) Multidrug ABC transporters in bacteria, Res Microbiol 170, 381-391.
46. Robey, R. W., Pluchino, K. M., Hall, M. D., Fojo, A. T., Bates, S. E., and Gottesman, M. M. (2018) Revisiting the role of ABC transporters in multidrug-resistant cancer, Nat Rev Cancer 18, 452-464.
47. Adams, K. N., Szumowski, J. D., and Ramakrishnan, L. (2014) Verapamil, and its metabolite norverapamil, inhibit macrophage-induced, bacterial efflux pump-mediated tolerance to multiple anti-tubercular drugs, J Infect Dis 210, 456-466.
48. Xu, J., Tasneen, R., Peloquin, C. A., Almeida, D. V., Li, S. Y., Barnes-Boyle, K., Lu, Y., and Nuermberger, E. (2018) Verapamil Increases the Bioavailability and Efficacy of Bedaquiline but Not Clofazimine in a Murine Model of Tuberculosis, Antimicrob Agents Chemother 62.

### Example IV—Measurement of Permeation Across the Mycomembrane in Live Mycobacteria

The general lack of permeability of small molecules observed for Mycobacterium tuberculosis (Mtb) is most commonly ascribed to its unique cell envelope. More specifically, the outer mycomembrane is hypothesized to be the principal determinant for access of antibiotics to their molecular targets. Despite this, there is limited information on the types of molecular scaffolds that can readily permeate past the mycomembrane of mycobacteria. To address this, we describe a novel assay that combines metabolic tagging of peptidoglycan and a fluorescent labeling chase step to measure the permeation of small molecules. The assay was robust and compatible with high-throughput analysis. In total, 1200 small molecules were tested, and we found a large range in the permeability profile. This assay platform will lay the foundation for medicinal chemistry efforts to improve uptake of both existing drugs and newly discovered compounds in mycobacteria. The methods described to can be generally adopted to species for which envelope permeability is also treatment-limiting, e.g., non-tuberculous mycobacteria (NTMs).

### Introduction

The Tuberculosis (TB) pandemic continues to impact large swarths of the global population with an estimated one-third of the world population being latently infected with Mycobacterium tuberculosis (Mtb), the causative agent of TB. The health burden caused by TB is immense. Yearly, 1.5 million people die from TB infections and only approximately ˜50% of patients are successfully treated from multi-drug resistant TB.^{1,2} TB infections are inherently difficult-to-treat due to the low number of antimycobacterial agents that effectively clear the pathogen from infected patients. Similar to Gram-negative bacteria, mycobacteria possess an outer membrane (OM) that encases the entire cell (FIG. 29A).^{3,4} This membrane (mycomembrane) serves as a formidable barrier that is thought to hinder the penetration of small molecules including antibiotics. As such, it has been implicated in endowing mycobacteria with a high level of intrinsic drug resistance to antimycobacterial agents.⁵

Given its location—lying at the interface between the potentially vulnerable inner components and the host—the mycomembrane is paramount in controlling the amount and types of molecules that translocate this barrier. Lack of permeation across the mycomembrane has long been hypothesized to be one of the primary reasons for the failure of antibiotics to accumulate in mycobacteria. While there is some variability across various mycobacterial species, there are several conserved components within the cellular envelope. The outer leaflet is composed primarily of polyacyltrehalose (PAT), diacyltrehalose (DAT), and trehalose dimycolate (TDM) molecules. Within the inner leaflet of the mycomembrane, there are a number of noncovalently linked lipids and lipoglycans, whose alkyl chains can range from C₆₀ to C₉₀. This waxy layer must pack tightly to fold the hydrocarbon chains into a prototypical 7-8 nm thick membrane.⁶ In doing so, the fluidity of the membrane is decreased⁵, thus also creating a less permeable barrier to small molecules.

The therapeutic effectiveness of most antimycobacterial agents is dependent on their ability to cross the mycomembrane to ultimately reach their cellular target. To this end, most antimycobacterial agents are relatively small and hydrophobic, whereas antimycobacterial drugs that are larger have been proposed to cross via a porin imbedded within the mycomembrane. A prominent example of such porins is MspA found in Mycobacterium smegmatis (Msm).⁷ Given the architecture of the mycobacterial cell wall, molecules that reach the peptidoglycan (PG) layer must have necessarily crossed the mycomembrane. Consequently, we reasoned that we could quantitatively probe the permeation of small molecules across the mycomembrane by quantifying the level of molecules that reach the PG scaffold.

PG is a mesh-like polymer made up of repeating disaccharides N-acetylglucosamine (GlcNAc) and N-acetylmuramic acid (MurNAc). Each MurNAc unit is connected to a short and unusual peptide (stem peptide) with the canonical sequence of L-Ala-D-iGlu-m-DAP-D-Ala-D-Ala (FIG. 29B).⁸ In all known bacteria, neighboring stem peptides are crosslinked by transpeptidases to endow the PG matrix with high levels of rigidity and integrity. Bacterial PG is an essential component of the bacterial cell wall, which makes its biosynthesis vulnerable to inhibition by small molecules such as β-lactams.^{9,10} We envisioned that site selective metabolic installation of a biorthogonal (“click chemistry”) handle within the PG scaffold of mycobacteria could be leveraged to assess the accumulation of small molecules across the outer mycomembrane. This workflow, Peptidoglycan Accessibility Click-Mediated AssessmeNt (PAC-MAN), can provide a platform to measure the permeation of any molecule that is modified with a small click handle. While a range of click chemistry handles have been developed to date that could potentially operate within the general workflow described, we focused on the combination of DiBenzoCycloOctyne (DBCO) and azide.^{11,12} This pair of reactive functional groups is biorthogonal and readily forms a stable triazole bond in the absence of metal catalysts based on strain promoted alkyne-azide cycloaddition (SPAAC).¹³ We expected that small molecules that readily permeate across the mycomembrane will react with PG-imbedded DBCO; therefore, there will be fewer available DBCO epitopes to react with an azide-tagged fluorophore, thus resulting in a decrease in cellular fluorescence.

### Materials and Methods

General materials. Dibenzocyclooctyne (DBCO)-NHS ester (BP-22231 1 g) was purchased from Broadpharm. 2-Chlorotrityl chloride resin, amino acids and coupling regents for solid phase synthesis were purchased from Chemimpex. 5-carboxy fluorescein, 6-azido-fluorescein, 6-azido rhodamine were purchased from Lumiprobe. 7-Azido-4-hydroxy coumarin, 7H9 broth, catalase from bovine liver, dextrose, and bovine serum albumin were purchased from Sigma Aldrich.

General cell culture. M. smegmatis strains mc² 155, ATCC 14468, PM2750 Δ5 were grown in 7H9 media with 0.5% glycerol, 0.05% tween 80, and 1×ADC enrichment (10×ADC, 5 g bovine serum albumin, 2 g dextrose, 3 mg catalase in 100 mL). M. smegmatis mc² 1255 was grown in the same media with 50 μg/mL streptomycin sulfate. Glycerol stocks were made using stationary phase cells in 30% glycerol and aliquots were stored in −80° C.

General method for bacterial peptidoglycan modification. M. smegmatis strains was inoculated from the glycerol stock to the according media and grown for 24 hours until 0.5-0.6 OD, tetrapeptide probes were added to the media and the cells were grown overnight to achieve stationary phase. The cells were harvested the next day and spun down for 2 min at 3000 g, washed with phosphate buffered saline with tween80 (PBST, PBS with 0.05% tween 80) two times and resuspended in PBST to yield DBCO-modified M. smegmatis cells for further labeling and other experiments.

DBCOtetra compared with stereo control and single amino acid DBCO. M. smegmatis mc² 155 was inoculated from stationary phase in 1:100 dilution to a fresh 7H₉ media with ADC. 25 μM Dap-DBCO, DBCOtetra or DBCOtera(L) were added to the culture tubes respectively, and the cells were grown for 36-40 hours until stationary phase. The cells were harvested and spun down for 2 min at 3000 g, washed with PBST two times and resuspended in PBST. To a 96-well plate added 100 μL cells pre well with 50 μM FAM-N3 in triplicate. The plate was incubated in 37° C. for 1 h and spun down for 2 min at 3000 g. The supernatant was decanted, and the pellets were washed with PBST for 2 times and fixed with 4% formaldehyde for 15 mins. The samples were then analyzed by Attune™ NxT Acoustic Focusing Cytometer.

Confirm for click chemistry on PG. M. smegmatis mc² 155 was inoculated from stationary phase in 1:100 dilution to a fresh 7H9 media with ADC. 25 μM DBCOtetra or FL-tetra were added to the culture tubes respectively, and the cells were grown for 36-40 hours until stationary phase. The cells were harvested and spun down for 2 min at 3000 g, washed with PBST two times and resuspended in PBST. To a 96-well plate added 100 uL cells pre well with 50 μM FAM-N3 or 5-carboxy fluorescein accordingly in triplicate. The plate was incubated in 37° C. for 1 h and spun down for 2 min at 3000 g. The supernatant was decanted, and the pellets were washed with PBST for 2 times and fixed with 4% formaldehyde for 15 mins. The samples were then analyzed by Attune™ NxT Acoustic Focusing Cytometer.

Different concentrations of DBCOtetra. M. smegmatis mc² 155 was inoculated from stationary phase in 1:100 dilution to a fresh 7H9 media with ADC. 5 μM, 10 μM or 25 μM DBCOtetra were added to the culture tubes respectively, and the cells were grown for 36-40 hours until stationary phase. The cells were harvested and spun down for 2 min at 3000 g, washed with PBST two times and resuspended in PBST. To a 96-well plate added 100 μL cells pre well with 50 μM FAM-N3 in triplicate. The plate was incubated in 37° C. for 1h and spun down for 2 min at 3000 g. The supernatant was decanted, and the pellets were washed with PBST for 2 times and fixed with 4% formaldehyde for 15 mins. The samples were then analyzed by Attune™ NxT Acoustic Focusing Cytometer.

Different concentration of FAM-N3 and different time. M. smegmatis mc² 155 was inoculated from stationary phase in 1:100 dilution to a fresh 7H9 media with ADC. 25 μM DBCOtetra were added to the culture tubes, and the cells were grown for 36-40 hours until stationary phase. The cells were harvested and spun down for 2 min at 3000 g, washed with PBST two times and resuspended in PBST. To a 96-well plate added 100 μL cells pre well with different concentration of FAM-N3 described in the main text, 9 wells each. The plate was incubated in 37° C., the cells were taken out at different time points and spun down for 2 min at 3000 g. The supernatant was decanted, and the pellets were washed with PBST for 2 times and fixed with 4% formaldehyde for 15 mins. The samples were then analyzed by Attune™ NxT Acoustic Focusing Cytometer.

Dye comparison. M. smegmatis mc² 155 was inoculated from stationary phase in 1:100 dilution to a fresh 7H9 media with ADC. 25 μM DBCOtetra were added to the culture tubes, and the cells were grown for 36-40 hours until stationary phase. The cells were harvested and spun down for 2 min at 3000 g, washed with PBST two times and resuspended in PBST. To a 96-well plate added 100 μL cells pre well with 50 μM FAM-N3, R110-N3 or 7-azido-4-hydroxy coumarin, respectively, in triplicate. The plate was incubated in 37° C. for 1 h and spun down for 2 min at 3000 g. The supernatant was decanted, and the pellets were washed with PBST for 2 times and fixed with 4% formaldehyde for 15 mins. The samples were then analyzed by Attune™ NxT Acoustic Focusing Cytometer. BL1 channel was used for FAM-N3 and R110-N3, VL1 channel was used for 7-azido-4-hydroxy coumarin.

Ldt knock down strain. M. smegmatis mc² 155 and PM2750 Δ5 were inoculated from the glycerol stock to the according media and grown for 24 hours until 0.5-0.6 OD, 25 μM DBCOtetra were added to the media, and the cells were grown overnight to achieve stationary phase. The cells were harvested and spinned down for 2 min at 3000 g, washed with PBST two times and resuspended in PBST. To a 96-well plate added 100 uL cells pre well with 50 μM FAM-N3 accordingly in triplicate. The plate was incubated in 37° C. for 1 h and spun down for 2 min at 3000 g. The supernatant was decanted, and the pellets were washed with PBST for 2 times and fixed with 4% formaldehyde for 15 mins. The samples were then analyzed by Attune™ NxT Acoustic Focusing Cytometer.

Ldt inhibition with meropenem. M. smegmatis mc² 155 was inoculated from the glycerol stock by 1 to 1000 dilution to the according media and grown for 24 hours until 0.5-0.6 OD. 25 μM DBCOtetra were added to the media, and the cells were aliquoted to a 96-well culture plate with different concentration of meropenem described in the main text, in triplicate respectively. The cells were then grown overnight to achieve stationary phase. The cells were harvested and spun down for 2 min at 3000 g, washed with PBST two times and resuspended in PBST. To a 96-well plate added 100 uL cells pre well with 50 μM FAM-N3 accordingly transferred from the culture plate. The plate was incubated in 37° C. for 1 h and spun down for 2 min at 3000 g. The supernatant was decanted, and the pellets were washed with PBST for 2 times and fixed with 4% formaldehyde for 15 mins. The samples were then analyzed by Attune™ NxT Acoustic Focusing Cytometer.

Competition with test molecules. To a 96-well plate added 100 uL DBCO-modified M. smegmatis mc² 155 cells pre well with 50 μM test molecules, in triplicate respectively. The plate was incubated in 37° C. for 2 h. The cells were spun down for 2 min at 3000 g. The supernatant was decanted, and 50 μM FAM-N3 was then added to each well, followed by incubation in 37° C. for 1 h. The cells were then spun down again, and the pellets were washed with PBST for 2 times and fixed with 4% formaldehyde for 15 mins. The samples were then analyzed by Attune™ NxT Acoustic Focusing Cytometer.

LLysN3 competition. To a 96-well plate added 100 uL DBCO-modified M. smegmatis mc² 155 cells pre well with different concentration of L-Lys-N3 described in the main text, in triplicate respectively. The plate was incubated in 37° C. for 2 h. The cells were spun down for 2 min at 3000 g. The supernatant was decanted, and 50 μM FAM-N3 was then added to each well, followed by incubation in 37° C. for 1 h. The cells were then spun down again, and the pellets were washed with PBST for 2 times and fixed with 4% formaldehyde for 15 mins. The samples were then analyzed by Attune® NxT Acoustic Focusing Cytometer.

Screen of the 48-plate library. M. smegmatis mc² 155 was inoculated from the glycerol stock by 1 to 1000 dilution to 50 mL 7H9 media with ADC in 250 mL Erlenmeyer flasks each day and grown for 24 hours until 0.5-0.6 OD. 25 μM DBCOtetra were added to the media, and the cells were grown overnight to achieve stationary phase. The cells were harvested and spun down for 2 min at 3000 g, washed with PBST two times and resuspended in PBST. To a 96-well plate added 100 uL cells pre well with 50 QM of each molecule each well. The plate was incubated in 37° C. for 2 h. The cells were spun down for 2 min at 3000 g. The supernatant was decanted, and 50 μM FAM-N3 was then added to each well, followed by incubation in 37° C. for 1 h. The cells were then spun down again, and the pellets were washed with PBST for 2 times and fixed with 4% formaldehyde for 15 mins. The samples were then analyzed by Attune™ NxT Acoustic Focusing Cytometer. Some of the molecules with less than 10% signal intensity were selected and tested in triplicates.

Ethidium bromide and Nile red whole-cell accumulation assay. To a Costar 96-well half area black opaque flat bottom plate added 100 uL DBCO-modified M. smegmatis cells each well with 5 μM ethidium bromide and 10 μM Nile red in triplicates, respectively. The fluorescent intensity at different time points were taken by a Synergy H₁ microplate reader for 90 min with 3 min intervals and continuously orbital shaking. Wavelengths ethidium bromide, excitation 530 nm, emission 590 nm; Nile red, excitation 540 nm, emission 630 nm.

PG isolation sacuflow M. smegmatis mc² 155 cells with and without DBCO-modification were added to a 96-well culture plate with 50 μM FAM-N3 and incubated in 37° C. for 1 h. Then the cells were spun down at 2700 g for 10 min and washed with PBST two times. The cell pellets were resuspended in 10 mM NH4HCO3 with protease inhibitor and bath sonicated for 30 mins. 10 μg/mL DNase and RNase were added to each well and the plate was placed in 4 for 1 h. The cell wall-enriched fraction was collected by centrifugation at 2700 g for 10 min. The pellet was then treated with PBS with 2% sodium dodecyl sulfate (SDS) and incubated at 50 for 1 h with shaking at 250 rpm. The suspension was spun down at 2700 g for 10 min. This treatment was repeated for two times. Then the resulting pellet was resuspended in PBS with 1% SDS and 0.1 mg/ml proteinase K. The suspension was then heated with boiling water for 1 h and then spun down at 2700 g for 10 min. The supernatant was discarded and the 1% SDS extraction step was repeated 2 times. The pellet was them washed twice with PBS and 4 times with deionized water to give mycolyl-arabinogalactan-peptidoglycan

Complex (MAPc). MAPc samples were taken from each well and analyzed by Attune™ NxT Acoustic Focusing Cytometer. The rest MAPc was resuspended in 0.5% KOH in methanol and incubated in 37° C. at 250 rpm for 4 days. The mixture was then washed with methanol 2 times and diethyl ether 2 times and air-dried to give arabinogalactan-peptidoglycan (AGPG). The resulting AGPG was resuspended in deionized water and samples were taken from each well and analyzed by Attune™ NxT Acoustic Focusing Cytometer. AGPG was digested with 0.05 N H₂SO₄ at 37° C. for 5 days and washed 4 times with deionized water to give insoluble PG. PG samples were taken from each well and analyzed by Attune™ NxT Acoustic Focusing Cytometer.

### Macrophage Cell Culture

### Confocal Imaging (for Whole Cell Labeling)

### Synthesis of DBCOtetra

To a 25 mL peptide synthesis vessel with 100 mg 2-Chlorotrityl chloride resin (0.142 mmol) resuspended in 15 mL dry dichloromethane, was added Fmoc-D-alanine (49 mg, 1.1 eq, 0.16 mmol), and diisopropylethylamine (DIEA, 4.4 eq, 0.11 mL, 0.62 mmol). The resin was shaken for 1 hour at room temperature and washed with methanol and dichloromethane (3 times and 15 mL each). Fmoc protecting group was removed with 6M piperazine in N, N-Dimethylformamide (DMF, 15 mL) for 30 min at room temperature and washed as before. Fmoc-L-Lys (Boc)-OH (3.0 eq, 0.20 g, 0.43 mmol), HBTU (3.0 eq, 0.16 g, 0.43 mmol), and DIEA (6.0 eq, 0.15 mL, 0.85 mmol) in DMF (15 mL) was added to the vessel and shaken for 2 h at room temperature. The Fmoc deprotection and coupling procedure was repeated using the same equivalent with Fmoc-D-glutamic acid □-amide and Fmoc-L-alanine.

DBCO was coupled on the N term of the tetra peptide on resin. 25-30 mg DBCO-NHS was dissolved in 1 mL dry DMF and added to the 25 mL peptide synthesis vessel with 100 mg equivalent 2-Chlorotrityl chloride resin with tetra peptide resuspended in 2 mL DMF. The resin was shaken overnight at room temperature and washed with methanol and dichloromethane (3 times and 15 mL each). The resin was then added 20% trifluoroacetic acid (TFA) in dichloromethane after wash and shaken in room temperature for 1 h. The liquid phase was filtered and concentrated with nitrogen flow and added icy ether to precipitate the peptide. The ether layer was decanted, and the resulting solid was washed with icy ether and air dried. The crude material was purified with reverse phased high performance liquid chromatography (RP-HPLC) using H₂O/MeOH to yield DBCO-tetra. The sample was analyzed for purity using a Waters 1525 with a Phenomenex Luna 5μ C8(2) 100 Å (250×4.6 mm) column; gradient elution with H₂O/CH₃CN. [QTOF-MS]: calculated for C36H46N7O8, 704.3402, found: (M+H)⁺ 704.3404.

Synthesis of DapDBCO To a 25 mL peptide synthesis vessel with 100 mg 2-Chlorotrityl chloride resin (0.142 mmol) resuspended in 15 mL dry dichloromethane, was added Na-Boc-Nβ-Fmoc-D-2,3-diaminopropionic acid (D-Dap, 67 mg, 1.1 eq, 0.16 mmol), and DIEA (4.4 eq, 0.11 mL, 0.62 mmol). The resin was shaken for 1 hour at room temperature and washed with methanol and dichloromethane (3 times and 15 mL each). Fmoc protecting group was removed with 6M piperazine in N, N-Dimethylformamide (DMF, 15 mL) for 30 min at room temperature and washed as before.

DBCO was coupled on the side chain of D-Dap on resin. 25-30 mg DBCO-NHS was dissolved in 1 mL dry DMF and added to the 25 mL peptide synthesis vessel with 100 mg equivalent 2-Chlorotrityl chloride resin with D-Dap resuspended in 2 mL DMF. The resin was shaken overnight at room temperature and washed with methanol and dichloromethane (3 times and 15 mL each). The resin was then added 20% trifluoroacetic acid (TFA) in dichloromethane after wash and shaken in room temperature for 1 h. The liquid phase was filtered and concentrated with nitrogen flow and added icy ether to precipitate the peptide. The ether layer was decanted, and the resulting solid was washed with icy ether and air dried. The crude material was purified with reverse phased high performance liquid chromatography (RP-HPLC) using H₂O/MeOH to yield D-Dap(DBCO). The sample was analyzed for purity using a Waters 1525 with a Phenomenex Luna 5μ C8(2) 100 Å (250×4.6 mm) column; gradient elution with H₂O/CH₃CN.

### Results and Discussion

We recently described conceptually analogous assay that reports on the accessibility of larger biopolymers to and within the PG scaffold in Staphylococcus aureus (S. aureus).¹⁴ S. aureus were treated with an unnatural amino acid (D-lysine) modified with an s-azide to label the entire PG scaffold with azide handles. Cells were then treated with fluorescently labeled biopolymers dually tagged—with DBCO and a fluorophore—to probe the accessibility of molecules to the cell surface. In the case of Gram-positive bacteria, the PG scaffold is fully exposed to the extracellular media and the biopolymers did not have to cross a membrane to be covalently anchored. Nonetheless, there are fundamental aspects of the assay that were adopted to establish PAC-MAN. In the case of PAC-MAN, we reasoned that the reactive handles needed to be reversed. The small molecules each included an azide tag that is small in size and minimally perturbs the physiochemical properties of the test small molecules. In turn, the DBCO handle was conjugated to the PG metabolic label that will promote to the tagging of the PG scaffold (FIG. 32A).

To design the metabolic labels of mycobacterial PG, we considered that the DBCO unit could be linked to the side chain of a single D-amino acid. During cell growth, exogenous single D-amino acids supplemented in the culture medium are swapped in the place of the D-alanine that occupies the 4^{th} or 5^{th} position within the stem peptide in the PG layer.^{3,15-30} As an alternative, the DBCO could be linked to a synthetic mimic of the stem peptide. We^{28, 31}, and others³²⁻³, recently showed that synthetic analogs of PG stem peptides can be crosslinked into the growing PG scaffold of live cells, including that of Msm and Mtb. Generally, we had previously found that the N-terminus of the tetrapeptide is much more tolerant to large conjugates than the sidechain of single D-amino acids.²⁰

We synthesized a single amino acid with a DBCO conjugated on the sidechain of D-Dap (D-DapD) and a tetrapeptide (TetD) with a DBCO conjugated on the N-terminus to empirically test the tolerance of the click handle on the metabolic label of live cells (FIG. 32). Msm cells were incubated with TetD, D-DapD, or vehicle to promote the tagging of the PG, washed to remove unincorporated tags, treated with azide-modified fluorescein (Fl-az), and cellular fluorescence was measured by flow cytometry (FIG. 32A). Satisfyingly, the background labeling of cells treated with vehicle was low, while cells treated with PG metabolic labels displayed considerably higher cellular fluorescence (FIG. 32C). As anticipated, the overall PG labeling levels observed with the single amino acid (D-DapD) were ˜40-fold above vehicle treated cells whereas labeling with TetD led to ˜85-fold fluorescence increase over vehicle, which became the primary PG tagging method going forward. A diastereomeric version of TetD was synthesized in which the C-terminal D-Ala was switched to L-Ala. We had previously shown that transpeptidases are sensitive to the stereocenter on the C-terminus, which is the amino acid that is removed during the first step of transpeptidation.^{25,31-33} Similarly, fluorescence levels for Msm treated with the stereocontrol agent were similar to vehicle treated cells (FIG. 32C). These results establish that Msm labeling with TetD led to a higher signal-to-noise ratio, and, therefore, TetD became the primary PG tagging method.

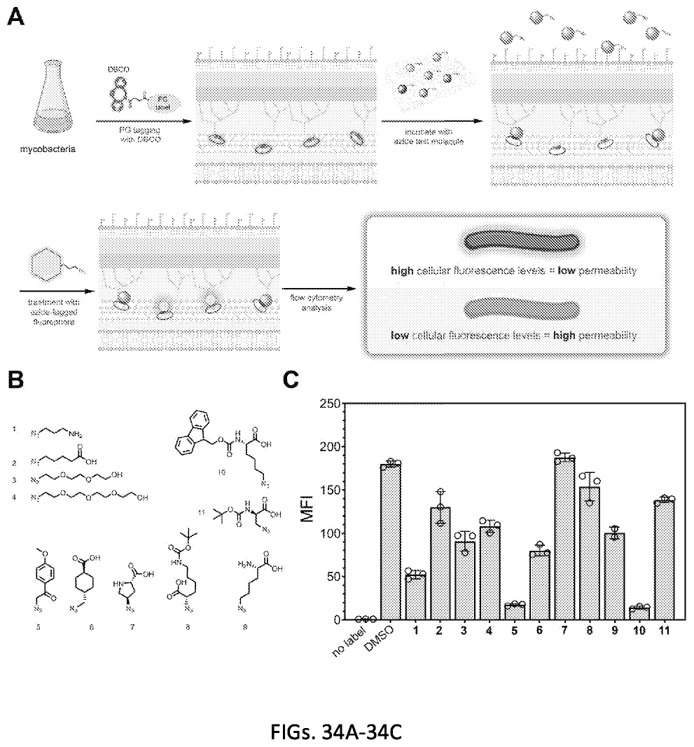

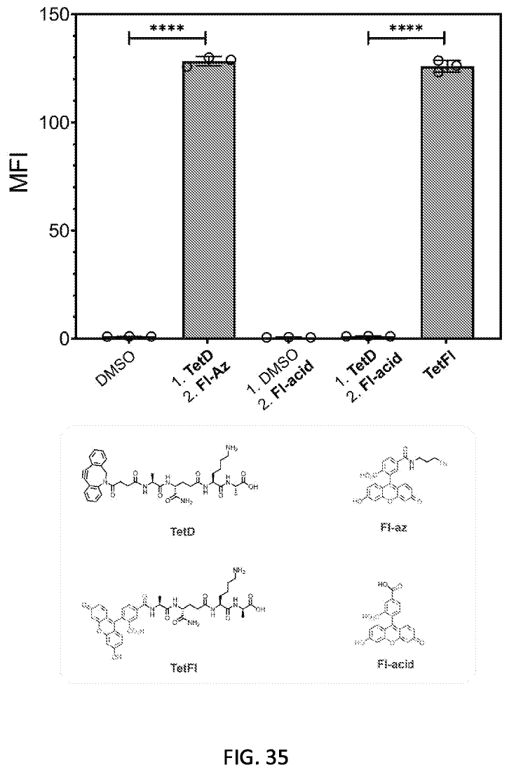

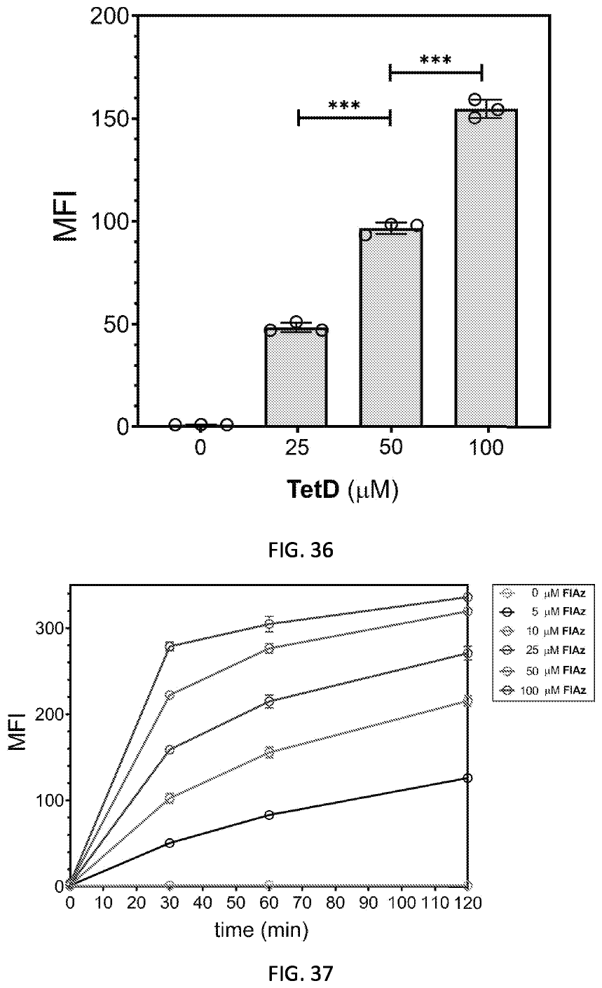

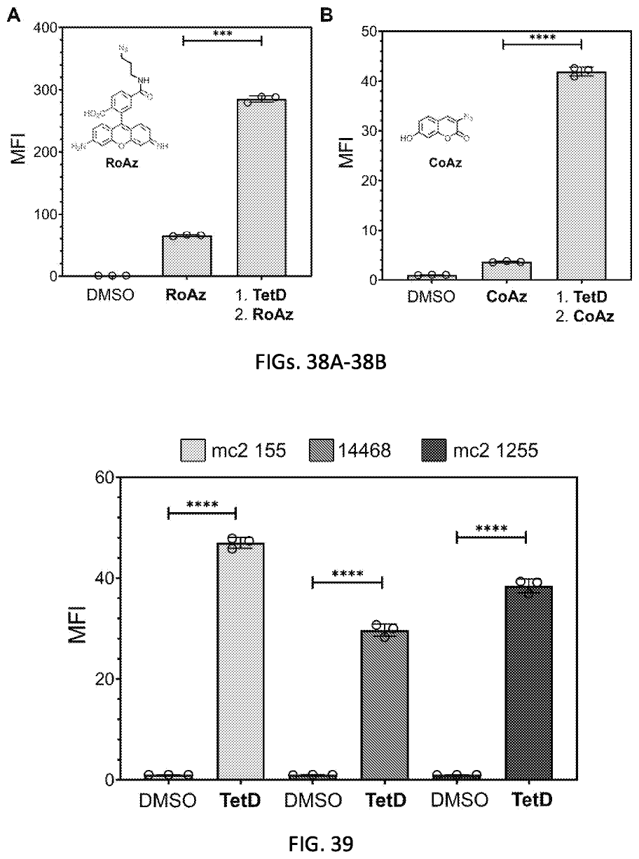

The necessity of DBCO for the cellular labeling was tested by incubating Msm cells with fluorescein alone. Cellular fluorescence signals were found to be background levels in cells not metabolically labeled or metabolically labeled with TetD when the azide group was not conjugated to fluorescein (FIG. 34). When the fluorescein was directly connected to the tetrapeptide (TetFl), fluorescence levels observed were nearly identical to cells labeled with TetD followed by Fl-az. These results indicate that fluorescence levels observed with cells treated with the TetD followed by the azide-linked fluorophore are reflective of SPAAC reactions within the PG scaffold and that the SPAAC reaction was nearly complete. A number of parameters were tested next to optimize labeling conditions. A titration experiment was performed with TetD and there was a concentration-dependent increase in cellular fluorescence was observed from 5 to 25 μM of TetD (FIG. 35). Additionally, a concentration-dependent and time-dependent increase in cellular fluorescence was observed with varying concentration of Fl-az (FIG. 36). These results indicated that 50 μM of Fl-az and an incubation period of 60 minutes led to a large increase in cellular fluorescence that should be sufficient for PAC-MAN. Understanding that the permeation of the fluorophore itself could be subject to the permeation barriers associated with the mycomembrane, we tested two additional azide-modified fluorophores: rhodamine 110 and coumarin (FIG. 37). While treatment of cells pre-labeled with TetD led to significant increases in cellular fluorescence with both fluorophores, they underperformed relative to Fl-az. In the case of rhodamine 110, fluorescence levels were higher than Fl-az, albeit with a lower fold increase over unlabeled cells. These results likely reflect non-specific binding of rhodamine 110 to Msm. For coumarin, there was a large fold increase over untreated cells despite the lower brightness of the fluorophore, which may reflect its smaller size and its turn-on configuration. Two other Msm strains were found to be similarly with a combination of TetD and Fl-az, an indication that the labeling strategy should be widely applicable to Msm (FIG. 38). Combined, these results establish that the fundamental first steps in PAC-MAN (metabolic labeling followed by SPAAC with a fluorophore) can operate in the context of non-pathogenic and pathogenic mycobacteria.

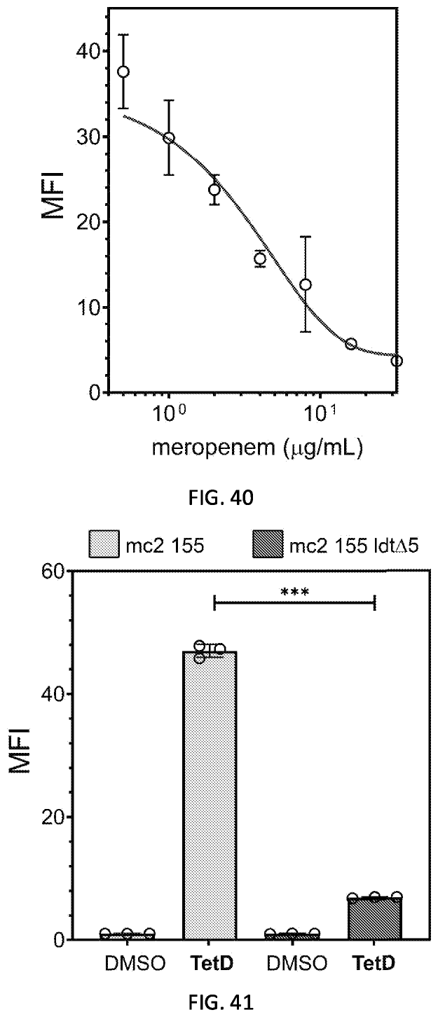

A number of subsequent experiments were performed to establish the localization of the DBCO epitopes within the cell walls of Msm. Two of these experiments performed were specifically designed to show that labeling levels were linked to PG transpeptidase activity, which are expected to crosslink the synthetic stem peptide mimic into the PG scaffold. A large number of β-lactam (e.g., penicillins, cephalosporins, and carbapenems) covalently inhibit PG transpeptidases, which should block the metabolic tagging by TetD.^{34,35} Msm cells were treated with increasing levels of meropenem in the presence of TetD and the fluorescence levels were analyzed as before. Critically, meropenem is known to inhibit L,D-transpeptidases (Ldt) that are expected to be the primary transpeptidase that processes the tetrapeptide mimic. A concentration dependent decrease in cellular fluorescence was observed, which is consistent with transpeptidase processing of TetD (FIG. 39). We next sought a genetic approach to delineate the labeling mechanism. An Ldt-deletion Msm strain, which has 5 of the Ldts are genetically deleted (ldtΔ5)^{25,36}, was likewise treated with TetD. Relative to the parental cells, fluorescence levels decreased ˜7-fold and are consistent with metabolic processing by Ldts (FIG. 40). Fluorescence levels were found to be higher than cells treated with meropenem, which may indicate that there remains TetD processing in ldtΔ5. Given that the third position on the synthetic peptide contains a lysine group, it is possible that some of the PG incorporation is also mediated by Penicillin Binding Proteins (PBPs) as acyl-acceptor strands. Confocal microscopy of whole cells and isolated sacculi were further used to delineate the localization of the fluorescent signal (FIG. 41).

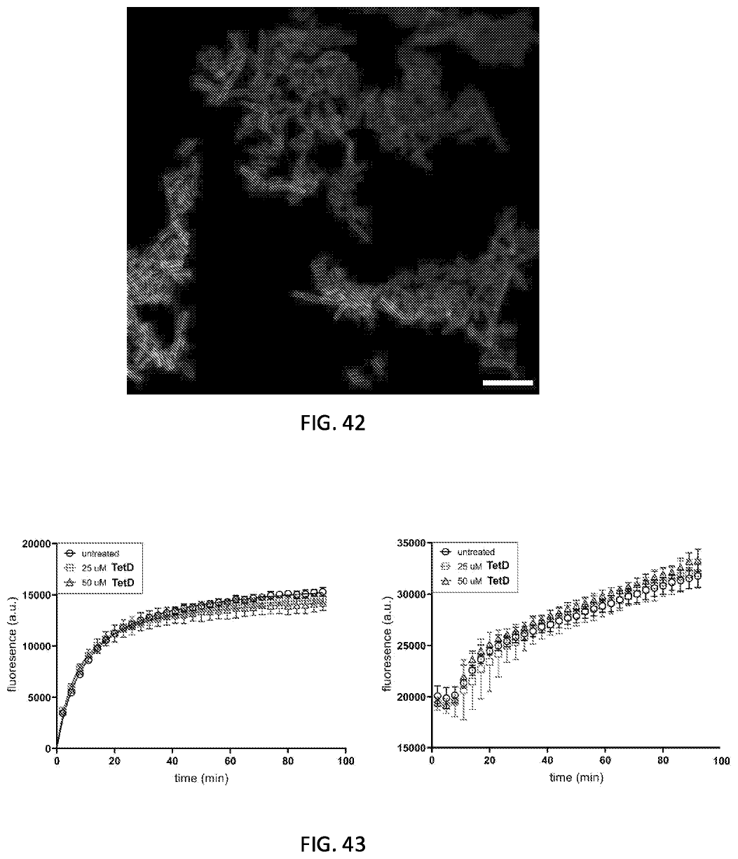

PG metabolic labeling has been widely utilized to gain a better understanding of cell wall biosynthesis and remodeling. Through these efforts, it has been found that, generally, these probes are not disruptive to the cell wall structure and do not alter the cellular viability. To directly probe the integrity of the mycomembrane, cells were analyzed for ethidium bromide (EtBr) accumulation/efflux and Nile red uptake by fluorescence measurements.³⁷⁻⁴¹ These dyes have been previously used as indicators of mycomembrane integrity as disruption to the mycomembrane will result in increased intracellular accumulation of these agents, which leads to higher cellular fluorescence. Msm cells were treated with vehicle or TetD, washed, stained with EtBr (hydrophilic dye) or Nile Red (hydrophobic dye), and whole cell accumulation was measured using a fluorescence plate reader. The results showed that in both cases, there was no significant change in the accumulation of the permeability probe when cells co-incubated TetD (FIG. 42). Taken together, these results confirm that Msm cells whose PG were metabolically tagged by treatment with TetD retain their cellular viability and, most importantly, the permeability profile of mycomembrane is not disrupted.

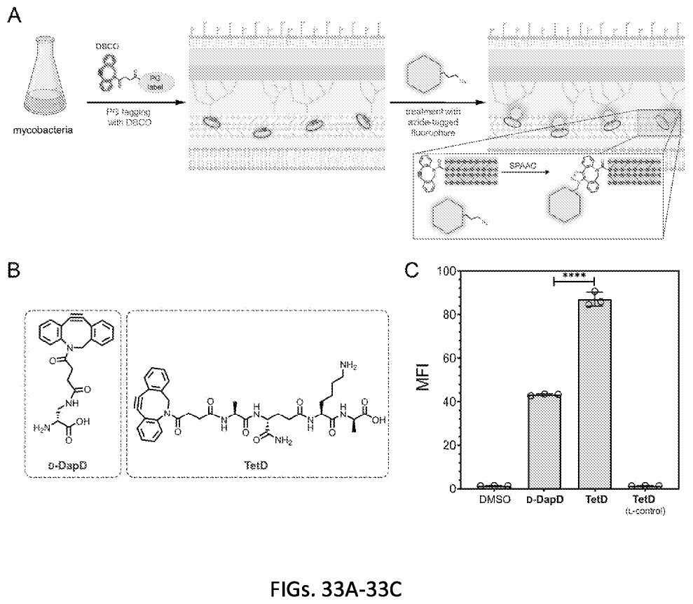

Next, we set out to benchmark the ability of the assay to report on the permeability of a set of test small molecules by performing a pilot screen with azide-conjugated compounds. The pilot screen was performed to demonstrate the robustness of the assay in a 96-well plate format and to characterize its reproducibility. PAC-MAN is initiated by metabolically tagging the PG of mycobacteria upon treatment with TetD (FIG. 33A). Batch treatment ensures similar PG labelling across the entire cell population. Cells are then washed to remove unincorporated molecules and bacterial cells and dispensed into individual wells in a 96-well plate. Each well contains a unique azide-modified small molecule. For molecules that permeate past the mycomembrane, they are expected to covalently react with the PG scaffold via SPAAC. Next, cells are treated with Fl-az to quantify the level of unreacted DBCO epitopes.⁴² Finally, cells are analyzed by flow cytometry to measure total cellular fluorescence.

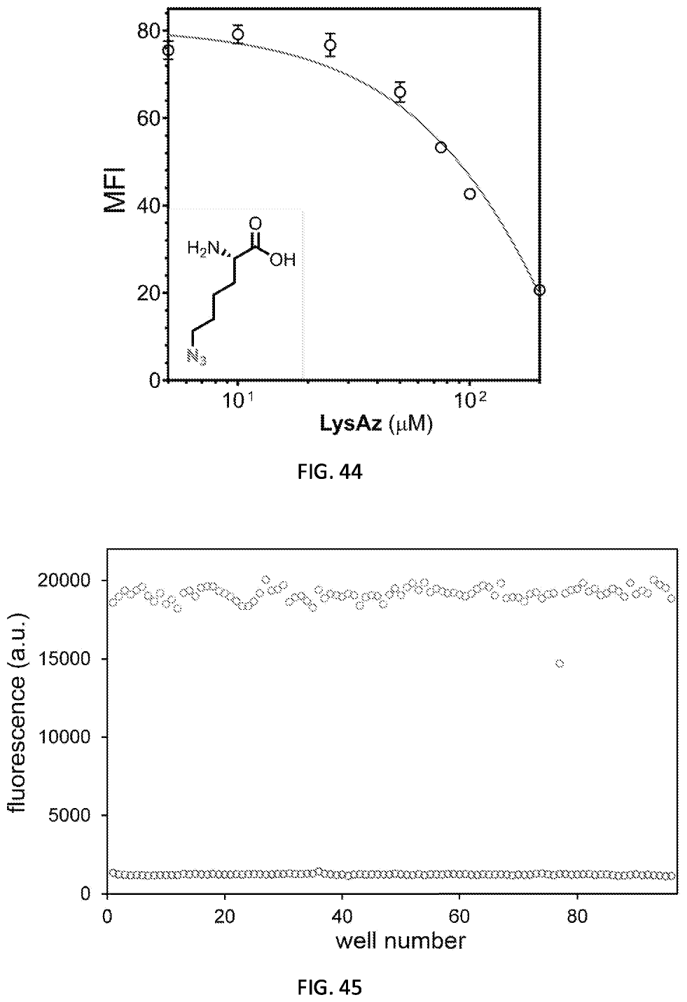

Screening 11 azide modified test molecules reveal that even within this small panel of molecules there was a significant dynamic range in signal (FIG. 33). Interesting patterns emerged such as the comparison of the Fmoc-protected Lys compared to the free amino acid. Despite the loss of a positive charge in the Fmoc-protected molecule, there was a significant increase in apparent permeability. This could indicate that the hydrophobicity could have a positive impact in permeation across the mycomembrane. A concentration dependent analysis of Lys revealed that it had an EC₅₀ of apparent permeation of 100 μM (FIG. 43). Critically, these results confirm that PAC-MAN can be used to compare the apparent permeation of molecules in Msm. Furthermore, 2 of the test molecules (a positive control and a negative control) were tested across an entire 96-well plate (FIG. 44) and it had a Z′ value of 0.89. There was a high level of stability observed, which indicates that PAC-MAN can be readily expanded to a high-throughput format.

### BIBLIOGRAPHY

1 Lienhardt, C. et al. Global tuberculosis control: lessons learnt and future prospects. Nat Rev Microbiol 10, 407-416, doi:10.1038/nrmicro2797 (2012).
2 Dartois, V. A. & Rubin, E. J. Anti-tuberculosis treatment strategies and drug development: challenges and priorities. Nat Rev Microbiol, doi:10.1038/s41579-022-00731-y (2022).
3 Siegrist, M. S., Swarts, B. M., Fox, D. M., Lim, S. A. & Bertozzi, C. R. Illumination of growth, division and secretion by metabolic labeling of the bacterial cell surface. FEMS Microbiol Rev 39, 184-202, doi:10.1093/femsre/fuu012 (2015).
4 Dulberger, C. L., Rubin, E. J. & Boutte, C. C. The mycobacterial cell envelope—a moving target. Nat Rev Microbiol 18, 47-59, doi:10.1038/s41579-019-0273-7 (2020).
5 Bansal-Mutalik, R. & Nikaido, H. Mycobacterial outer membrane is a lipid bilayer and the inner membrane is unusually rich in diacyl phosphatidylinositol dimannosides. Proc Natl Acad Sci USA 111, 4958-4963, doi:10.1073/pnas.1403078111 (2014).
6 Hoffmann, C., Leis, A., Niederweis, M., Plitzko, J. M. & Engelhardt, H. Disclosure of the mycobacterial outer membrane: cryo-electron tomography and vitreous sections reveal the lipid bilayer structure. Proc Natl Acad Sci USA 105, 3963-3967, doi:10.1073/pnas.0709530105 (2008).
7 Faller, M., Niederweis, M. & Schulz, G. E. The structure of a mycobacterial outer-membrane channel. Science 303, 1189-1192, doi:10.1126/science.1094114 (2004).
8 Egan, A. J. F., Errington, J. & Vollmer, W. Regulation of peptidoglycan synthesis and remodelling. Nat Rev Microbiol 18, 446-460, doi:10.1038/s41579-020-0366-3 (2020).
9 Royet, J. & Dziarski, R. Peptidoglycan recognition proteins: pleiotropic sensors and effectors of antimicrobial defences. Nat Rev Microbiol 5, 264-277, doi:10.1038/nrmicrol620 (2007).
10 Wolf, A. J. & Underhill, D. M. Peptidoglycan recognition by the innate immune system. Nat Rev Immunol 18, 243-254, doi:10.1038/nri.2017.136 (2018).
11 Baskin, J. M. et al. Copper-free click chemistry for dynamic in vivo imaging. Proc Natl Acad Sci USA 104, 16793-16797, doi:10.1073/pnas.0707090104 (2007).
12 Jewett, J. C., Sletten, E. M. & Bertozzi, C. R. Rapid Cu-free click chemistry with readily synthesized biarylazacyclooctynones. J Am Chem Soc 132, 3688-3690, doi:10.1021/ja100014q (2010).
13 Agard, N. J., Prescher, J. A. & Bertozzi, C. R. A strain-promoted [3+2] azide-alkyne cycloaddition for covalent modification of biomolecules in living systems. J Am Chem Soc 126, 15046-15047, doi:10.1021/ja044996f (2004).
14 Ferraro, N. J., Kim, S., Im, W. & Pires, M. M. Systematic Assessment of Accessibility to the Surface of Staphylococcus aureus. ACS Chem Biol, doi:10.1021/acschembio.1c00604 (2021).
Kuru, E. et al. In Situ probing of newly synthesized peptidoglycan in live bacteria with fluorescent D-amino acids. Angew Chem Int Ed Engl 51, 12519-12523, doi:10.1002/anie.201206749 (2012).
16 Kuru, E., Tekkam, S., Hall, E., Brun, Y. V. & Van Nieuwenhze, M. S. Synthesis of fluorescent D-amino acids and their use for probing peptidoglycan synthesis and bacterial growth in situ. Nat Protoc 10, 33-52, doi:10.1038/nprot.2014.197 nprot.2014.197 [pii] (2015).
17 Siegrist, M. S. et al. (D)-Amino acid chemical reporters reveal peptidoglycan dynamics of an intracellular pathogen. ACS Chem Biol 8, 500-505, doi:10.1021/cb3004995 (2013).
18 Lebar, M. D. et al. Reconstitution of peptidoglycan cross-linking leads to improved fluorescent probes of cell wall synthesis. J Am Chem Soc 136, 10874-10877, doi:10.1021/ja505668f (2014).
19 Lupoli, T. J. et al. Transpeptidase-mediated incorporation of D-amino acids into bacterial peptidoglycan. J Am Chem Soc 133, 10748-10751, doi:10.1021/ja2040656 (2011).
20 Fura, J. M., Kearns, D. & Pires, M. M. D-Amino Acid Probes for Penicillin Binding Protein-based Bacterial Surface Labeling. J Biol Chem 290, 30540-30550, doi:10.1074/jbc.M115.683342 (2015).
21 Fura, J. M., Sabulski, M. J. & Pires, M. M. D-amino acid mediated recruitment of endogenous antibodies to bacterial surfaces. ACS Chem Biol 9, 1480-1489, doi:10.1021/cb5002685 (2014).
22 Pidgeon, S. E. & Pires, M. M. Cell Wall Remodeling of Staphylococcus aureus in Live Caenorhabditis elegans. Bioconjug Chem 28, 2310-2315, doi:10.1021/acs.bioconjchem.7b00363 (2017).
23 Pidgeon, S. E. et al. Metabolic Profiling of Bacteria by Unnatural C-terminated D-Amino Acids. Angew Chem Int Ed Engl 54, 6158-6162, doi:10.1002/anie.201409927 (2015).
24 Apostolos, A. J., Pidgeon, S. E. & Pires, M. M. Remodeling of Cross-bridges Controls Peptidoglycan Cross-linking Levels in Bacterial Cell Walls. ACS Chem Biol 15, 1261-1267, doi:10.1021/acschembio.0c00002 (2020).
25 Pidgeon, S. E. et al. L,D-Transpeptidase Specific Probe Reveals Spatial Activity of Peptidoglycan Cross-Linking. ACS Chem Biol 14, 2185-2196, doi:10.1021/acschembio.9b00427 (2019).
26 Fura, J. M., Pidgeon, S. E., Birabaharan, M. & Pires, M. M. Dipeptide-Based Metabolic Labeling of Bacterial Cells for Endogenous Antibody Recruitment. ACS Infect Dis 2, 302-309, doi:10.1021/acsinfecdis.6b00007 (2016).
27 Caparros, M., Aran, V. & de Pedro, M. A. Incorporation of S-[3H]methyl-D-cysteine into the peptidoglycan of ether-treated cells of Escherichia coli. FEMS Microbiol Lett 72, 139-146, doi:10.1016/0378-1097(92)90519-t (1992).
28 Cava, F., Lam, H., de Pedro, M. A. & Waldor, M. K. Emerging knowledge of regulatory roles of D-amino acids in bacteria. Cell Mol Life Sci 68, 817-831, doi:10.1007/s00018-010-0571-8 (2011).
29 de Pedro, M. A., Quintela, J. C., Holtje, J. V. & Schwarz, H. Murein segregation in Escherichia coli. J Bacteriol 179, 2823-2834, doi:10.1128/jb.179.9.2823-2834.1997 (1997).
30 Schouten, J. A. et al. Fluorescent reagents for in vitro studies of lipid-linked steps of bacterial peptidoglycan biosynthesis: derivatives of UDPMurNAc-pentapeptide containing d-cysteine at position 4 or 5. Mol Biosyst 2, 484-491, doi:10.1039/b607908c (2006).
31 Apostolos, A. J., Ferraro, N. J., Dalesandro, B. E. & Pires, M. M. SaccuFlow: A High-Throughput Analysis Platform to Investigate Bacterial Cell Wall Interactions. ACS Infect Dis 7, 2483-2491, doi:10.1021/acsinfecdis.1c00255 (2021).
32 Apostolos, A. J. et al. Facile Synthesis and Metabolic Incorporation of m-DAP Bioisosteres Into Cell Walls of Live Bacteria. ACS Chem Biol 15, 2966-2975, doi:10.1021/acschembio.0c00618 (2020).
33 Apostolos, A. J. & Pires, M. M. Impact of crossbridge structure on peptidoglycan crosslinking: A synthetic stem peptide approach. Methods Enzymol 665, 259-279, doi:10.1016/bs.mie.2021.11.019 (2022).
34 Dubee, V. et al. Inactivation of Mycobacterium tuberculosis l,d-transpeptidase LdtMt(1) by carbapenems and cephalosporins. Antimicrob Agents Chemother 56, 4189-4195, doi:10.1128/AAC.00665-12 (2012).
35 Cordillot, M. et al. In vitro cross-linking of Mycobacterium tuberculosis peptidoglycan by L,D-transpeptidases and inactivation of these enzymes by carbapenems. Antimicrob Agents Chemother 57, 5940-5945, doi:10.1128/AAC.01663-13 (2013).
36 Sanders, A. N., Wright, L. F. & Pavelka, M. S. Genetic characterization of mycobacterial L,D-transpeptidases. Microbiology (Reading) 160, 1795-1806, doi:10.1099/mic.0.078980-0 (2014).
37 Rodrigues, L., Ramos, J., Couto, I., Amaral, L. & Viveiros, M. Ethidium bromide transport across Mycobacterium smegmatis cell-wall: correlation with antibiotic resistance. BMC Microbiol 11, 35, doi:10.1186/1471-2180-11-35 (2011).
38 Papadopoulos, A. O., Ealand, C., Gordhan, B. G., VanNieuwenhze, M. & Kana, B. D. Characterisation of a putative M23-domain containing protein in Mycobacterium tuberculosis. PLoS One 16, e0259181, doi:10.1371/journal.pone.0259181 (2021).
39 Chuang, Y. M. et al. Deficiency of the novel exopolyphosphatase Rv1026/PPX2 leads to metabolic downshift and altered cell wall permeability in Mycobacterium tuberculosis. mBio 6, e02428, doi:10.1128/mBio.02428-14 (2015).
40 Campodonico, V. L., Rifat, D., Chuang, Y. M., Ioerger, T. R. & Karakousis, P. C. Altered Mycobacterium tuberculosis Cell Wall Metabolism and Physiology Associated With RpoB Mutation H526D. Front Microbiol 9, 494, doi:10.3389/fmicb.2018.00494 (2018).
41 Bisson, G. P. et al. Upregulation of the phthiocerol dimycocerosate biosynthetic pathway by rifampin-resistant, rpoB mutant Mycobacterium tuberculosis. J Bacteriol 194, 6441-6452, doi:10.1128/JB.01013-12 (2012).
42 Shieh, P. et al. CalFluors: A Universal Motif for Fluorogenic Azide Probes across the Visible Spectrum. J Am Chem Soc 137, 7145-7151, doi:10.1021/jacs.5b02383 (2015).

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. A live cell assay to determine permeability of test molecules to and/or within the peptidoglycan (PG) scaffold of bacteria cells comprising:
a) provide live bacteria cells that comprise PG with a reactive epitope;
b) contact said cells of a) with one or more test molecules, wherein the one or more test molecules has a reactive handle that reacts with reactive epitope in the PG, wherein the test molecule has a reporter molecule; and
c) measure the amount of reporter molecule, wherein an increase in reporter molecule levels as compared to a control where the cells where not contacted with the test molecule correlates with permeation of said one or more test molecules to and/or within the PG scaffold.

2. A live cell assay to determine permeability of test molecules to and/or within the peptidoglycan (PG) scaffold of bacteria cells comprising:
a) provide live bacteria cells that comprise PG with a reactive handle;
b) contact said cells of a) with one or more test molecules, wherein the one or more test molecules has a reactive epitope that binds with reactive handle;
c) contact the cells of b) with a reporter molecule that is conjugated to a reactive epitope; and
d) measure the amount of reporter molecule, wherein a decrease in reporter molecule levels as compared to a control where the cells where not contacted with the test molecule correlates with permeation of said one or more test molecules to and/or within the PG scaffold.

3. The method of claim 1, wherein prior to b) the cells are cultured with an inhibitor of wall teichoic acid (WTA) biosynthesis.

4. The method of claim 3, wherein the inhibitor is tunicamycin.

5. The method of claim 1, wherein prior to b) the cells are cultured with positively charged, branched polyethylenimine (BPEI).

6. The method of claim 1, wherein the PG is covalently linked to the reactive epitope.

7. The method of claim 6, wherein the PG is covalently linked to the reactive epitope by culturing said cells with said reactive epitope for a time to allow the cells to incorporate the PG-reactive epitope into the cell's PG scaffold.

8. The method of claim 1, wherein the reactive epitope is part of a stem peptide for culturing with said cells.

9. The method of claim 8, wherein the stem peptide is 1, 2, 3, 4, 5, 6, 7, 8, 9 or 10 amino acids long and can be used as a PG building block by the cells.

10. The method of claim 8, wherein the reactive epitope is on the N-terminus, C-terminus or internal in the stem peptide.

11. The method of claim 1, wherein the reactive epitope is D-amino acid.

12. The method of claim 1, wherein the reactive epitope comprises a thiol or azide group.

13. The method of claim 1, wherein the reactive handle, test compound or reactive epitope is conjugated to the reporter molecule either directly or by a linker.

14. The method of claim 13, wherein the linker is at least one PEG.

15. The method of claim 1, wherein the reporter molecule is a fluorophore.

16. The method of claim 15, wherein the fluorophore is fluorescein, AF488, AF647, BODIPY, Cy5, rhodamine 110, TAMRA, Cy5.5, Cy7, Cy7.5 or coumarin.

17. The method of claim 1, wherein the reactive handle is maleimide or DiBenzoCycloOctyne (DBCO).

18. The method of claim 2, wherein the reactive handle is a modified amino acid or stem peptide.

19. The method of claim 18, wherein the stem peptide is 1, 2, 3, 4, 5, 6, 7, 8, 9 or 10 amino acids long.

20. (canceled)

21. The method of claim 18, wherein the reactive handle is on the N-terminus, C-terminus or internal in the stem peptide.

22. The method claim 18, wherein one or more of the amino acids is a D-amino acid.

23. The method of claim 18, wherein the reactive handle comprises a DBCO.

24. The method of claim 1, wherein the bacteria are gram-positive bacteria, gram-negative bacteria, mycobacteria or a combination thereof.

25-28. (canceled)

29. A maleimide or DiBenzoCycloOctyne (DBCO) compound comprising a modified amino acid or stem peptide.

30. The maleimide or DBCO compound of claim 29, wherein the stem peptide is 1, 2, 3, 4, 5, 6, 7, 8, 9 or 10 amino acids long.

31. (canceled)

32. The maleimide or DBCO compound of claim 29, wherein the maleimide or DBCO is on the N-terminus, C-terminus or internal in the stem peptide.

33. The maleimide or DBCO compound of claim 29, wherein one or more of the amino acids is a D-amino acid.

34. (canceled)

35. The maleimide or DBCO compound of claim 29, wherein the compound comprises the following structure:

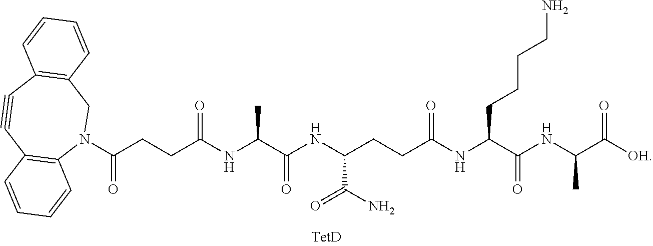 [SMILES: C[C@H](NC(=O)CCC(=O)N1Cc2ccccc2C#Cc2ccccc21)C(=O)N[C@H](CCC(=O)N[C@@H](CCCCN)C(=O)N[C@H](C)C(=O)O)C(N)=O]

36. The maleimide or DBCO compound of claim 29, wherein the compound comprises the following structure: 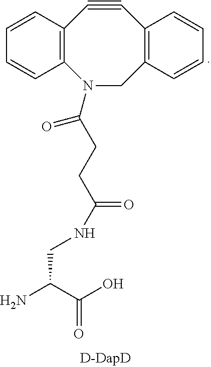 [SMILES: N[C@H](CNC(=O)CCC(=O)N1Cc2ccccc2C#Cc2ccccc21)C(=O)O ; "."]
